(19) United States
(12) Patent Application Publication
(10) Pub. No.: US 2022/0296599 A1
(43) Pub. Date: Sep. 22, 2022

(54) Pharmaceutical composition comprising histone deacetylase inhibitor and methotrexate

(71) Applicant: Chong Kun Dang Pharmaceutical Corp., Seoul (KR)

(72) Inventors: Young II Choi, Gyeonggi-do (KR); Nina Ha, Gyeonggi-do (KR); Daekwon Bae, Gyeonggi-do (KR); Dong Hyeon Suh, Gyeonggi-do (KR)

(21) Appl. No.: 17/284,591

(22) PCT Filed: Oct. 11, 2019
(86) PCT No.: PCT/KR2019/013389

§ 371 Date: Apr. 12, 2021

(30) Foreign Application Priority Data
Oct. 12, 2018 (KR) 10 2018-0122155

(51) Int. Cl. A61K 31/519; A61K 31/506; A61K 31/5377; A61K 31/496; A61K 31/551; A61K 31/397; A61K 31/40; A61K 31/553; A61K 31/4439; A61K 31/444; A61K 31/4433; A61K 31/357; A61P 19/02

(52) U.S. Cl. CPC A61K 31/519; A61K 31/506; A61K 31/5377; A61K 31/496; A61K 31/551; A61K 31/397; A61P 19/02; A61K 31/553; A61K 31/4439; A61K 31/444; A61K 31/4433; A61K 31/357; A61K 31/40

(57) ABSTRACT

The present invention relates to a pharmaceutical composition for preventing or treating inflammatory rheumatic diseases, comprising a histone deacetylase inhibitor and methotrexate as an effective component; a treatment method using the composition; and a use of the composition in preparing a drug for treating inflammatory rheumatic diseases, wherein the pharmaceutical composition according to the present invention shows an excellent effect on preventing or treating inflammatory rheumatic diseases.

### TECHNICAL FIELD

The present invention relates to a pharmaceutical composition for preventing or treating inflammatory rheumatic diseases, comprising a histone deacetylase inhibitor and methotrexate as effective components; a treatment method using the composition; and a use of the composition in preparing a drug for preventing or treating inflammatory rheumatic diseases.

### BACKGROUND ART

Rheumatic diseases involve various painful disorders, which have an influence on motor systems including soft tissues, particularly surrounding joints, muscles, connective tissues, articulations and bones, wherein inflammations or autoimmune reactions contribute to causes of many rheumatic diseases. Such conditions, which are generally referred to inflammatory rheumatic diseases, include the arthritis, osteoarthritis, etc. of various origins in a non-limiting way.

Out of rheumatic diseases, rheumatoid arthritis (RA) is a representative systemic chronic autoimmune disease, which starts with inflammations of synovial membrane, and then does damage to joints and bones to cause deformation and disability thereof. Rheumatoid arthritis mainly starts from small joints of hands and feet, and then progresses into large joints, wherein it may also make an invasion into other organs to cause extra-articular manifestations such as pericarditis, pulmonary fibrosis, peripheral neuritis, etc. A direct cause of rheumatoid arthritis has not been clarified yet, but it is known that rheumatoid arthritis occurs due to a combination of genetic susceptibility and environmental factors, and an age of onset is mainly distributed in the thirties to fifties, thus causing a severe influence on patients' economic activities.

Now, there is no effective therapeutic agent for rheumatoid arthritis. Instead, drugs for alleviating symptoms have been prescribed to relieve inflammations and pains. However, even in case of using such therapeutic agents, the disease repeatedly recurs after remission and thus gradually causes structural damages to joints. A recent trend of treatment for rheumatoid arthritis has made a progress in the direction of controlling symptoms and minimizing structural damages to joints by means of early diagnosis and early prescription of disease modifying anti-rheumatic drugs (DMARDs).

Methotrexate (MTX) has been used as a first-line therapeutic agent for rheumatoid arthritis. A medicinal effect of MTX is evaluated in three months after its prescription. Then, if the disease is not improved, other DMARDs are further prescribed to minimize symptoms and damages to joints. However, MTX shows a decrease in therapeutic efficacy, in that 30 to 40% of patients having taken the drug fail to achieve a medicinal effect thereof within three months after its prescription. MTX has also a problem of causing hepatotoxicity upon its long-term use. A recent trend is an increase in the cases of prescribing biologics such as CTLA4-Fc, TNFα neutralizing antibody or the like in combination as a second-line therapy after MTX. However, biologics have a disadvantage, in that they are expensive and administered as an injection, thus causing inconvenience. Due to characteristics of an antibody drug, it is inevitable that biologics result in occurrence of neutralizing antibodies, which reportedly becomes a cause of resistance to the biologics. Also, NSAIDs, steroid preparations, hydroxychloroquine, sulfasalazine, leflunomide, etc., which serve as second and third-line oral therapeutic agents, show a quick effect, but are recommended for short-term use rather than long-term use due to rising concerns about various side effects. Tofacitinib (Xeljanz®, Pfizer, Jak3 inhibitor), which is an oral therapeutic agent for arthritis released in 2014, exhibits an excellent medicinal effect, but causes an increase in LDL cholesterol levels (5 mg: 15%, 10 mg: 30%) within one month after start of intake. And Tofacitinib has some problems, in that it arouses high concerns about thromboembolism, infection and carcinogenicity, while having a strong immunosuppression effect at the same time.

Accordingly, although there have been reportedly many therapeutics agents for inflammatory rheumatic diseases so far, most of the therapeutic agents have a disadvantage of side effects as well as a medicinal effect. Thus, there is an urgent need for developing an effective therapeutic agent with less side effects. In particular, a greater importance has been put on developing a safe oral therapeutic agent for rheumatoid arthritis, which may be used in combination with MTX and may be also used early on for those patients who do not respond to MTX, a first-line prescription drug.

### DISCLOSURE OF INVENTION

### Technical Problem

An objective of the present invention is to provide a pharmaceutical composition for preventing or treating inflammatory rheumatic diseases, comprising a histone deacetylase inhibitor and methotrexate.

Other objective of the present invention is to provide a method for preventing or treating inflammatory rheumatic diseases, including administering a therapeutically effective amount of the composition.

Another objective of the present invention is to provide a use of the composition in preparing a drug for preventing or treating inflammatory rheumatic diseases.

### Solution to Problem

This is described in detail as follows. Meanwhile, each description and embodiment disclosed in the present invention may be also applied to other descriptions and embodiments thereof, respectively. In other words, all the combinations of various elements disclosed in the present invention fall within the scope of the present invention. Also, it cannot be seen that the scope of the present invention is limited to the specific description described below.

The present invention provides a pharmaceutical composition for preventing or treating inflammatory rheumatic diseases, containing a histone deacetylase inhibitor and methotrexate.

In the present invention, the histone deacetylase inhibitor may be a compound represented by a following formula I, optical isomers thereof or pharmaceutically acceptable salts thereof:

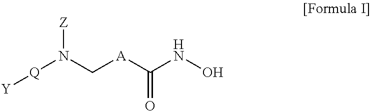

wherein

A

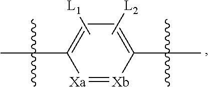

Xa and Xb are each independently CH or N,

L₁ and L₂ are each independently hydrogen, halogen, —CF₃, or —C₁₋₃ straight or branched chain alkyl,

Q is C(═O), S(═O)₂, S(═O) or C(═NH), and

Y is selected from a following group:

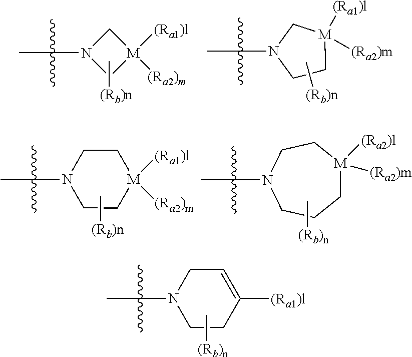

M is C, N, O, S or S(═O)₂ (here, if M is C, l and m are 1; if M is N, l is 1 and m is 0; and if M is O, S or S(═O)₂, l and m are 0),

Rₐ₁ and Rₐ₂ are each independently hydrogen; hydroxy; —C₁₋₄ straight or branched chain alkyl, which is unsubstituted or substituted with at least one halogen; —C₁₋₄ straight or branched chain alcohol; benzhydryl; —C₁₋₄ straight or branched chain alkyl, which is substituted with a saturated or unsaturated five to seven-membered heterocyclic compound having one to three heteroatoms out of N, O or S as a ring member (here, the heterocyclic compound may be unsubstituted or at least one hydrogen may be optionally substituted with OH, OCH₃, CH₃, CH₂CH₃ or halogen); a saturated or unsaturated five to seven-membered heterocyclic compound having one to three heteroatoms out of N, O or S as a ring member (here, the heterocyclic compound may be unsubstituted or at least one hydrogen may be optionally substituted with OH, OCH₃, CH₃, CH₂CH₃ or halogen); phenyl, wherein it is unsubstituted or at least one hydrogen is substituted with halogen, C₁₋₄ alkoxy, C₁₋₂alkyl or hydroxy; benzyl, wherein it is unsubstituted or at least one hydrogen is substituted with halogen, C₁₋₄ alkoxy, C₁₋₂alkyl or hydroxy; —S(═O)₂CH₃; halogen; —C₁₋₆ straight or branched chain alkoxy; —C₂₋₆ alkoxyalkyl; —C(═O)Rₓ, wherein Rₓ is C₁₋₃ straight or branched chain alkyl or C₃₋₁₀ cycloalkyl;

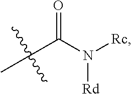

wherein R_{c} and R_{d} are independently hydrogen or C₁₋₃ straight or branched chain alkyl;

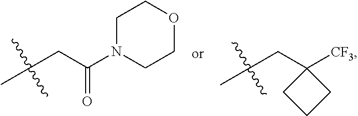 [SMILES: *CC(=O)N1CCOCC1 ; *CC1(C(F)(F)F)CCC1]

n is an integer of 0, 1 or 2,

R_{b} is hydrogen; hydroxy; —C₁₋₆ straight or branched chain alkyl, wherein it is unsubstituted or at least one hydrogen is substituted with halogen; —C(═O)CH₃; —C₁₋₄ straight or branched chain hydroxyalkyl; —C₁₋₆ straight or branched chain alkoxy; —C₂₋₆ straight or branched chain alkoxyalkyl; —CF₃; halogen; or

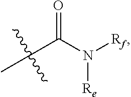

Rₑ and R_{f} are each independently hydrogen or —C₁₋₃ straight or branched chain alkyl, and

Z is selected from a following group:

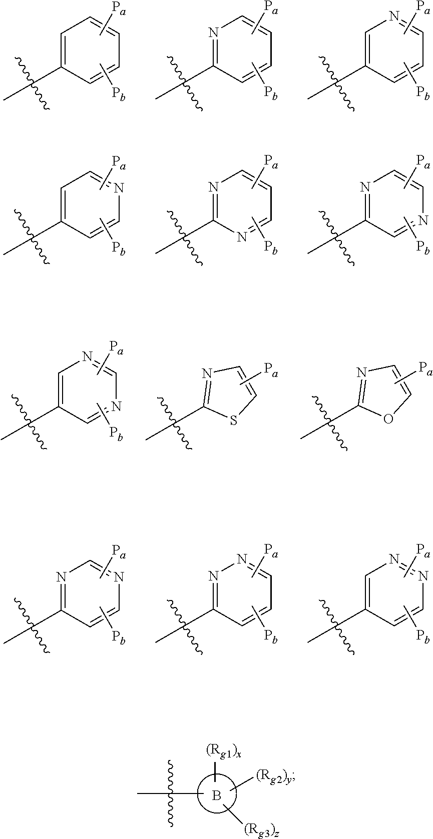

hydrogen; hydroxy; —C₁₋₄ straight or branched chain alkyl, wherein it is unsubstituted or at least one hydrogen is substituted with halogen; halogen; —CF₃; —OCF₃; —CN; —C₁₋₆ straight or branched chain alkoxy; —C₂₋₆ straight or branched chain alkyl alkoxy; —CH₂F; or —C₁₋₃ alcohol,

wherein

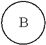

is a ring selected from phenyl, pyridine, pyrimidine, thiazole, indole, indazole, piperazine, quinoline, furan, tetrahydropyridine, piperidine or a following group:

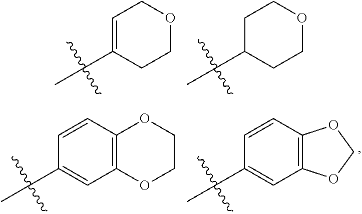

x, y and z are each independently an integer of 0 or 1, and

R_{g1}, R_{g2} and R_{g3} are each independently selected from hydrogen; hydroxy; —C₁₋₃ alkyl; —CF₃; —C₁₋₆ straight or branched chain alkoxy; —C₂₋₆ straight or branched chain alkyl alkoxy; —C(═O)CH₃; —C₁₋₄ straight or branched chain hydroxyalkyl; —N(CH₃)₂; halogen; phenyl; —S((═O)₂)CH₃; or a following group:

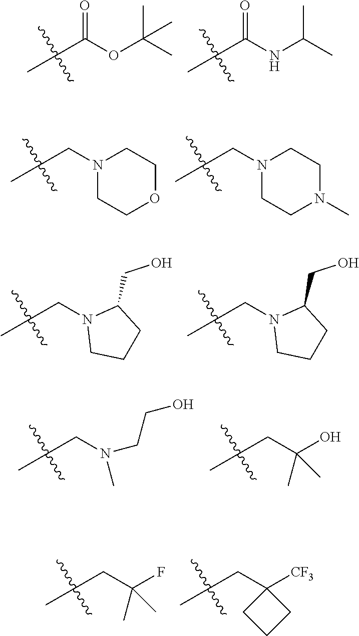

In the present invention, “halogen” is represented by F, Cl, Br or I.

In an embodiment, the histone deacetylase inhibitor may be a compound represented by a following formula II, optical isomers thereof or pharmaceutically acceptable salts thereof:

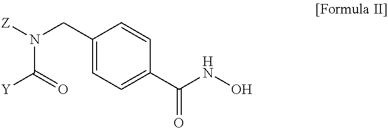

wherein

Y is selected from a following group:

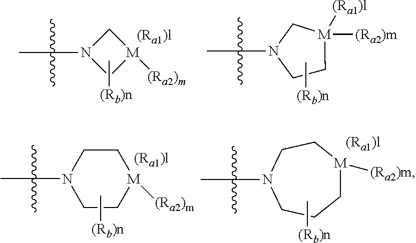

each of M, l, m, n, Rₐ₁, Rₐ₂ and R_{b} is the same with a definition of Formula I above,

Z is

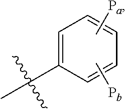

and

Pₐ and P_{b} are each independently hydrogen; hydroxy; —C₁₋₄ straight or branched chain alkyl, wherein it is unsubstituted or at least one hydrogen is substituted with halogen; halogen; —CF₃; —OCF₃; —CN; —C₁₋₆ straight or branched chain alkoxy; —C₂₋₆ straight or branched chain alkyl alkoxy; —CH₂F; or —C₁₋₃ alcohol.

According to a specific embodiment of the present invention, the compound represented by the formula I above is as described in following tables 1 to 12.

TABLE 1

| Com- | |
|---|---|
| pound | Structural formula |
| 252 | 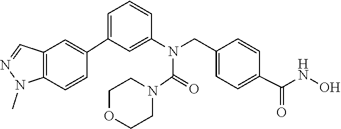 [SMILES: Cn1ncc2cc(-c3cccc(N(Cc4ccc(C(=O)NO)cc4)C(=O)N4CCOCC4)c3)ccc21] |
| 253 | 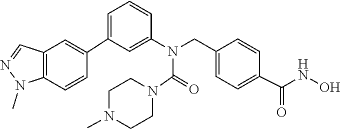 [SMILES: CN1CCN(C(=O)N(Cc2ccc(C(=O)NO)cc2)c2cccc(-c3ccc4c(cnn4C)c3)c2)CC1] |
| 254 | 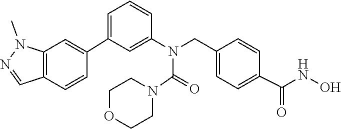 [SMILES: Cn1ncc2ccc(-c3cccc(N(Cc4ccc(C(=O)NO)cc4)C(=O)N4CCOCC4)c3)cc21] |
| 255 | 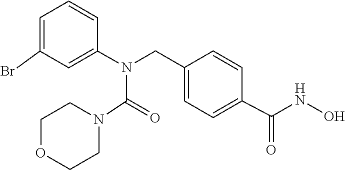 [SMILES: O=C(NO)c1ccc(CN(C(=O)N2CCOCC2)c2cccc(Br)c2)cc1] |
| 256 | 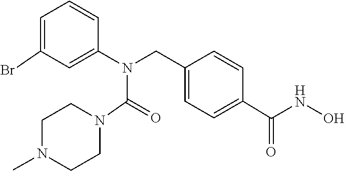 [SMILES: CN1CCN(C(=O)N(Cc2ccc(C(=O)NO)cc2)c2cccc(Br)c2)CC1] |
| 260 | 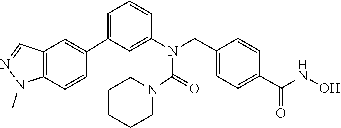 [SMILES: Cn1ncc2cc(-c3cccc(N(Cc4ccc(C(=O)NO)cc4)C(=O)N4CCCCC4)c3)ccc21] |
| 261 | 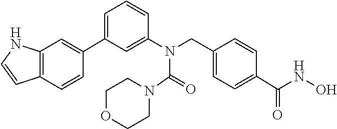 [SMILES: O=C(NO)c1ccc(CN(C(=O)N2CCOCC2)c2cccc(-c3ccc4cc[nH]c4c3)c2)cc1] |
| 262 | 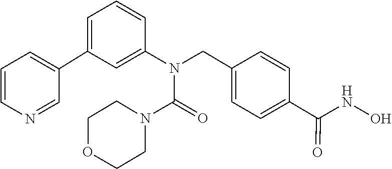 [SMILES: O=C(NO)c1ccc(CN(C(=O)N2CCOCC2)c2cccc(-c3cccnc3)c2)cc1] |
| 263 | 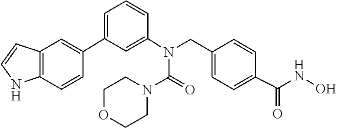 [SMILES: O=C(NO)c1ccc(CN(C(=O)N2CCOCC2)c2cccc(-c3ccc4[nH]ccc4c3)c2)cc1] |
| 279 | 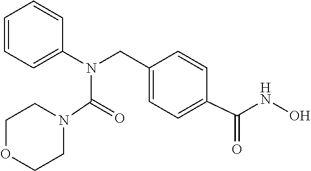 [SMILES: O=C(NO)c1ccc(CN(C(=O)N2CCOCC2)c2ccccc2)cc1] |
| 280 | 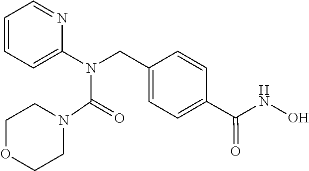 [SMILES: O=C(NO)c1ccc(CN(C(=O)N2CCOCC2)c2ccccn2)cc1] |
| 281 | 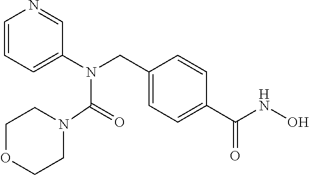 [SMILES: O=C(NO)c1ccc(CN(C(=O)N2CCOCC2)c2cccnc2)cc1] |
| 308 | 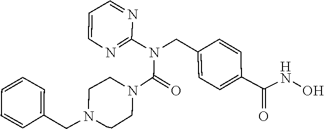 [SMILES: O=C(NO)c1ccc(CN(C(=O)N2CCCN(Cc3ccccc3)CC2)c2ncccn2)cc1] |
| 311 | 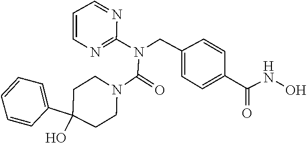 [SMILES: O=C(NO)c1ccc(CN(C(=O)N2CCC(O)(c3ccccc3)CC2)c2ncccn2)cc1] |

TABLE 2

| Com- | |
|---|---|
| pound | Structural formula |
| 312 | 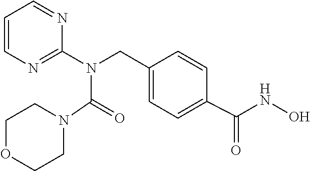 [SMILES: O=C(NO)c1ccc(CN(C(=O)N2CCOCC2)c2ncccn2)cc1] |
| 313 | 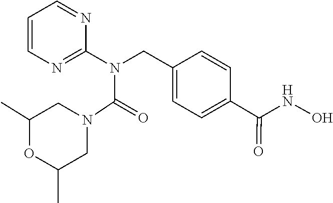 [SMILES: CC1CN(C(=O)N(Cc2ccc(C(=O)NO)cc2)c2ncccn2)CC(C)O1] |
| 329 | 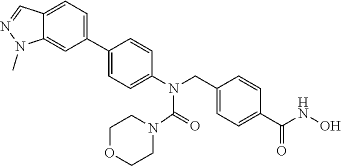 [SMILES: Cn1ncc2ccc(-c3ccc(N(Cc4ccc(C(=O)NO)cc4)C(=O)N4CCOCC4)cc3)cc21] |
| 330 | 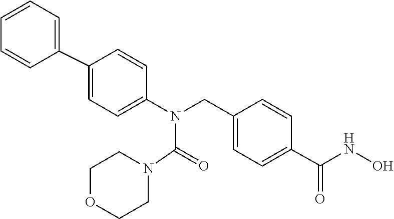 [SMILES: O=C(NO)c1ccc(CN(C(=O)N2CCOCC2)c2ccc(-c3ccccc3)cc2)cc1] |
| 331 | 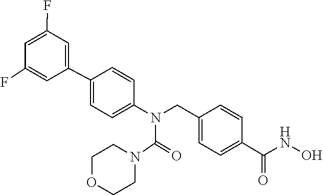 [SMILES: O=C(NO)c1ccc(CN(C(=O)N2CCOCC2)c2ccc(-c3cc(F)cc(F)c3)cc2)cc1] |
| 332 | 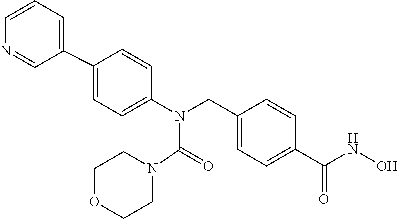 [SMILES: O=C(NO)c1ccc(CN(C(=O)N2CCOCC2)c2ccc(-c3cccnc3)cc2)cc1] |
| 333 | 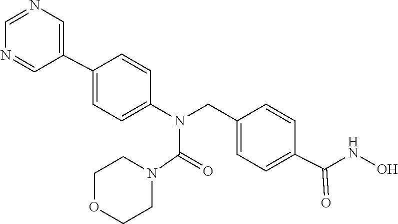 [SMILES: O=C(NO)c1ccc(CN(C(=O)N2CCOCC2)c2ccc(-c3cncnc3)cc2)cc1] |
| 334 | 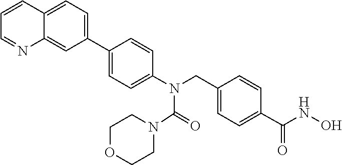 [SMILES: O=C(NO)c1ccc(CN(C(=O)N2CCOCC2)c2ccc(-c3ccc4cccnc4c3)cc2)cc1] |
| 335 | 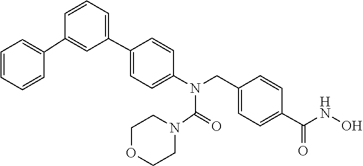 [SMILES: O=C(NO)c1ccc(CN(C(=O)N2CCOCC2)c2ccc(-c3cccc(-c4ccccc4)c3)cc2)cc1] |
| 336 | 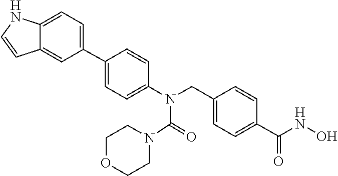 [SMILES: O=C(NO)c1ccc(CN(C(=O)N2CCOCC2)c2ccc(-c3ccc4[nH]ccc4c3)cc2)cc1] |
| 337 | 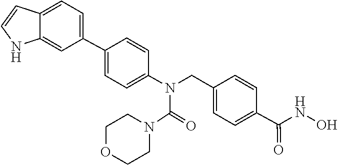 [SMILES: O=C(NO)c1ccc(CN(C(=O)N2CCOCC2)c2ccc(-c3ccc4cc[nH]c4c3)cc2)cc1] |
| 338 | 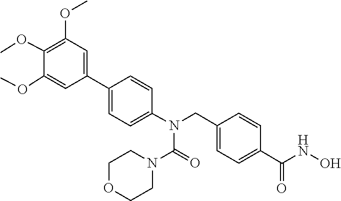 [SMILES: COc1cc(-c2ccc(N(Cc3ccc(C(=O)NO)cc3)C(=O)N3CCOCC3)cc2)cc(OC)c1OC] |
| 339 | 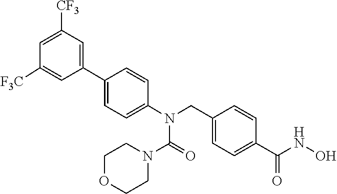 [SMILES: O=C(NO)c1ccc(CN(C(=O)N2CCOCC2)c2ccc(-c3cc(C(F)(F)F)cc(C(F)(F)F)c3)cc2)cc1] |
| 340 | 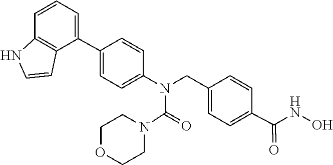 [SMILES: O=C(NO)c1ccc(CN(C(=O)N2CCOCC2)c2ccc(-c3cccc4[nH]ccc34)cc2)cc1] |

TABLE 3

| Com- | |
|---|---|
| pound | Structural formula |
| 341 | 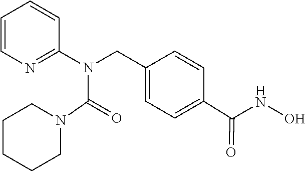 [SMILES: O=C(NO)c1ccc(CN(C(=O)N2CCCCC2)c2ccccn2)cc1] |
| 342 | 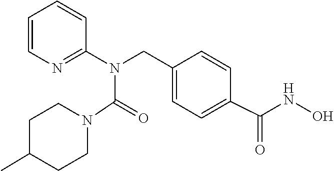 [SMILES: CC1CCN(C(=O)N(Cc2ccc(C(=O)NO)cc2)c2ccccn2)CC1] |
| 343 | 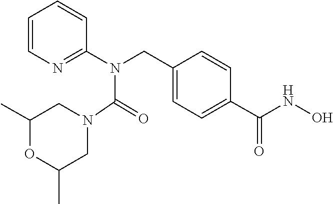 [SMILES: CC1CN(C(=O)N(Cc2ccc(C(=O)NO)cc2)c2ccccn2)CC(C)O1] |
| 352 | 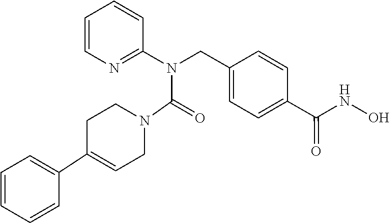 [SMILES: O=C(NO)c1ccc(CN(C(=O)N2CC=C(c3ccccc3)CC2)c2ccccn2)cc1] |
| 353 | 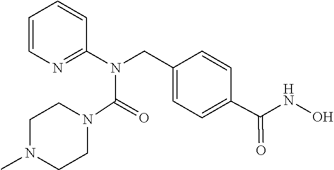 [SMILES: CN1CCN(C(=O)N(Cc2ccc(C(=O)NO)cc2)c2ccccn2)CC1] |
| 354 | 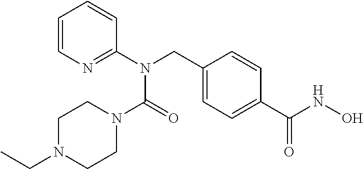 [SMILES: CCN1CCN(C(=O)N(Cc2ccc(C(=O)NO)cc2)c2ccccn2)CC1] |
| 355 | 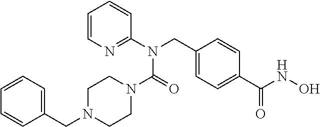 [SMILES: O=C(NO)c1ccc(CN(C(=O)N2CCN(Cc3ccccc3)CC2)c2ccccn2)cc1] |
| 356 | 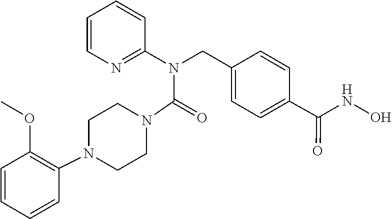 [SMILES: COc1ccccc1N1CCN(C(=O)N(Cc2ccc(C(=O)NO)cc2)c2ccccn2)CC1] |
| 357 | 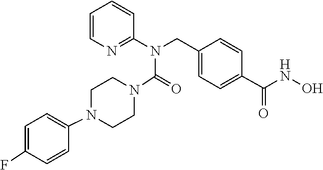 [SMILES: O=C(NO)c1ccc(CN(C(=O)N2CCN(c3ccc(F)cc3)CC2)c2ccccn2)cc1] |
| 358 | 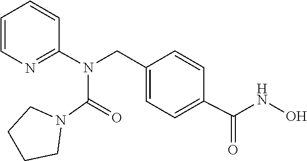 [SMILES: O=C(NO)c1ccc(CN(C(=O)N2CCCC2)c2ccccn2)cc1] |
| 370 | 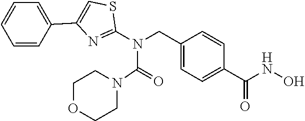 [SMILES: O=C(NO)c1ccc(CN(C(=O)N2CCOCC2)c2nc(-c3ccccc3)cs2)cc1] |
| 371 | 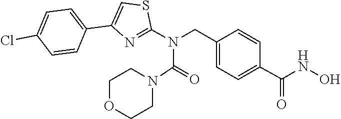 [SMILES: O=C(NO)c1ccc(CN(C(=O)N2CCOCC2)c2nc(-c3ccc(Cl)cc3)cs2)cc1] |
| 372 | 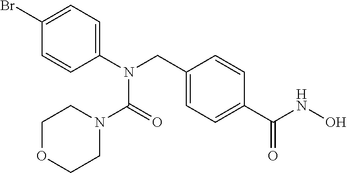 [SMILES: O=C(NO)c1ccc(CN(C(=O)N2CCOCC2)c2ccc(Br)cc2)cc1] |
| 374 | 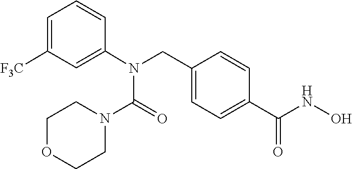 [SMILES: O=C(NO)c1ccc(CN(C(=O)N2CCOCC2)c2cccc(C(F)(F)F)c2)cc1] |

TABLE 4

| Com- | |
|---|---|
| pound | Structural formula |
| 376 | 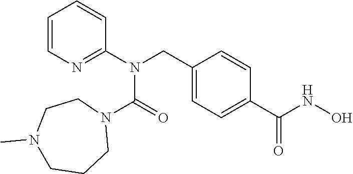 [SMILES: CN1CCCN(C(=O)N(Cc2ccc(C(=O)NO)cc2)c2ccccn2)CC1] |
| 377 | 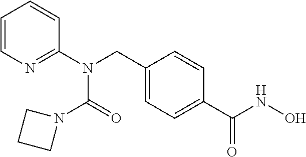 [SMILES: O=C(NO)c1ccc(CN(C(=O)N2CCC2)c2ccccn2)cc1] |
| 379 | 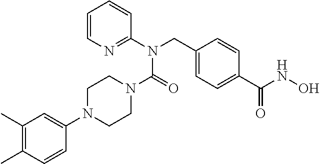 [SMILES: Cc1ccc(N2CCN(C(=O)N(Cc3ccc(C(=O)NO)cc3)c3ccccn3)CC2)cc1C] |
| 380 | 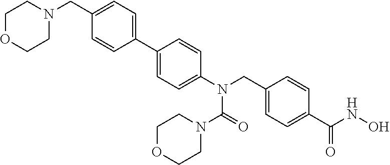 [SMILES: O=C(NO)c1ccc(CN(C(=O)N2CCOCC2)c2ccc(-c3ccc(CN4CCOCC4)cc3)cc2)cc1] |
| 381 | 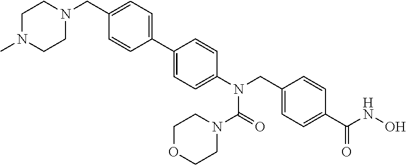 [SMILES: CN1CCN(Cc2ccc(-c3ccc(N(Cc4ccc(C(=O)NO)cc4)C(=O)N4CCOCC4)cc3)cc2)CC1] |
| 382 | 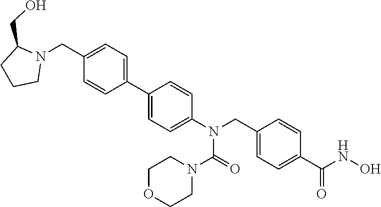 [SMILES: O=C(NO)c1ccc(CN(C(=O)N2CCOCC2)c2ccc(-c3ccc(CN4CCC[C@H]4CO)cc3)cc2)cc1] |
| 383 | 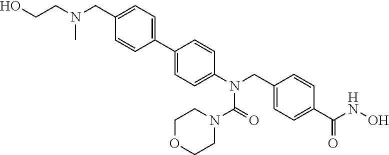 [SMILES: CN(CCO)Cc1ccc(-c2ccc(N(Cc3ccc(C(=O)NO)cc3)C(=O)N3CCOCC3)cc2)cc1] |
| 385 | 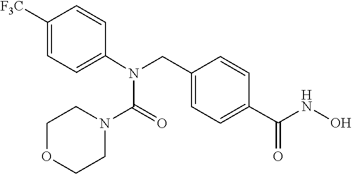 [SMILES: O=C(NO)c1ccc(CN(C(=O)N2CCOCC2)c2ccc(C(F)(F)F)cc2)cc1] |
| 386 | 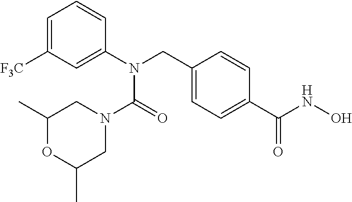 [SMILES: CC1CN(C(=O)N(Cc2ccc(C(=O)NO)cc2)c2cccc(C(F)(F)F)c2)CC(C)O1] |
| 389 | 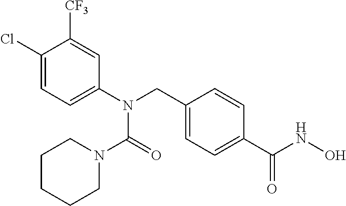 [SMILES: O=C(NO)c1ccc(CN(C(=O)N2CCCCC2)c2ccc(Cl)c(C(F)(F)F)c2)cc1] |
| 390 | 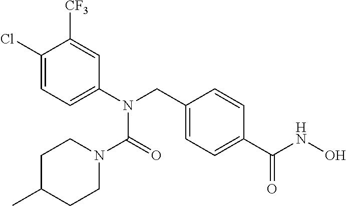 [SMILES: CC1CCN(C(=O)N(Cc2ccc(C(=O)NO)cc2)c2ccc(Cl)c(C(F)(F)F)c2)CC1] |
| 391 | 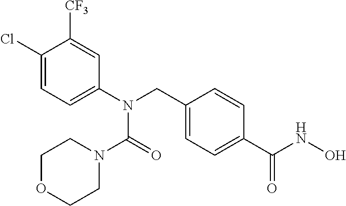 [SMILES: O=C(NO)c1ccc(CN(C(=O)N2CCOCC2)c2ccc(Cl)c(C(F)(F)F)c2)cc1] |
| 392 | 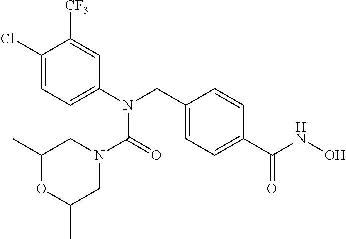 [SMILES: CC1CN(C(=O)N(Cc2ccc(C(=O)NO)cc2)c2ccc(Cl)c(C(F)(F)F)c2)CC(C)O1] |
| 393 | 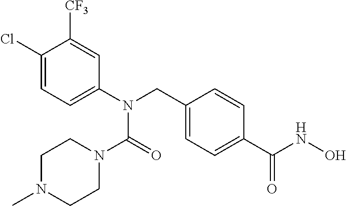 [SMILES: CN1CCN(C(=O)N(Cc2ccc(C(=O)NO)cc2)c2ccc(Cl)c(C(F)(F)F)c2)CC1] |

TABLE 5

| Com- | |
|---|---|
| pound | Structural formula |
| 394 | 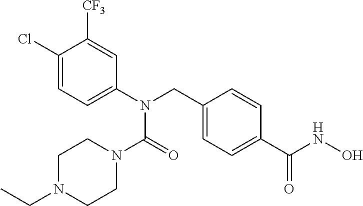 [SMILES: CCN1CCN(C(=O)N(Cc2ccc(C(=O)NO)cc2)c2ccc(Cl)c(C(F)(F)F)c2)CC1] |
| 395 | 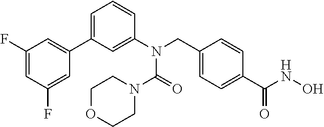 [SMILES: O=C(NO)c1ccc(CN(C(=O)N2CCOCC2)c2cccc(-c3cc(F)cc(F)c3)c2)cc1] |
| 396 | 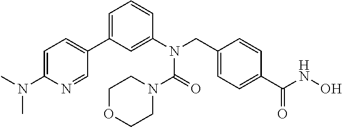 [SMILES: CN(C)c1ccc(-c2cccc(N(Cc3ccc(C(=O)NO)cc3)C(=O)N3CCOCC3)c2)cn1] |
| 397 | 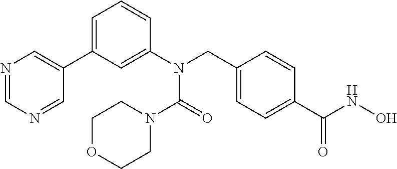 [SMILES: O=C(NO)c1ccc(CN(C(=O)N2CCOCC2)c2cccc(-c3cncnc3)c2)cc1] |
| 398 | 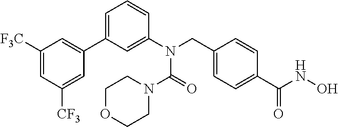 [SMILES: O=C(NO)c1ccc(CN(C(=O)N2CCOCC2)c2cccc(-c3cc(C(F)(F)F)cc(C(F)(F)F)c3)c2)cc1] |
| 399 | 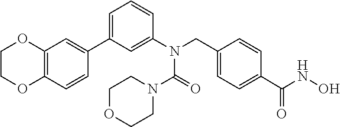 [SMILES: O=C(NO)c1ccc(CN(C(=O)N2CCOCC2)c2cccc(-c3ccc4c(c3)OCCO4)c2)cc1] |
| 400 | 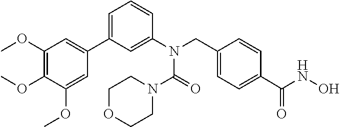 [SMILES: COc1cc(-c2cccc(N(Cc3ccc(C(=O)NO)cc3)C(=O)N3CCOCC3)c2)cc(OC)c1OC] |
| 401 | 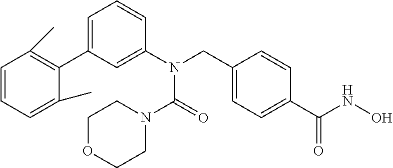 [SMILES: Cc1cccc(C)c1-c1cccc(N(Cc2ccc(C(=O)NO)cc2)C(=O)N2CCOCC2)c1] |
| 402 | 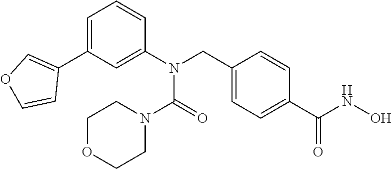 [SMILES: O=C(NO)c1ccc(CN(C(=O)N2CCOCC2)c2cccc(-c3ccoc3)c2)cc1] |
| 403 | 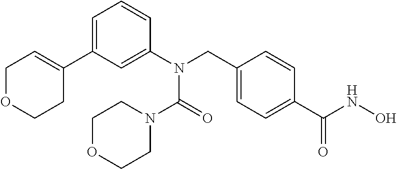 [SMILES: O=C(NO)c1ccc(CN(C(=O)N2CCOCC2)c2cccc(C3=CCOCC3)c2)cc1] |
| 404 | 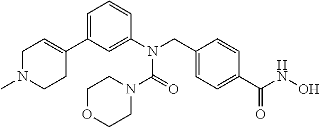 [SMILES: CN1CC=C(c2cccc(N(Cc3ccc(C(=O)NO)cc3)C(=O)N3CCOCC3)c2)CC1] |
| 405 | 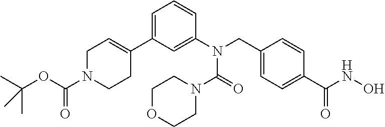 [SMILES: CC(C)(C)OC(=O)N1CC=C(c2cccc(N(Cc3ccc(C(=O)NO)cc3)C(=O)N3CCOCC3)c2)CC1] |
| 413 | 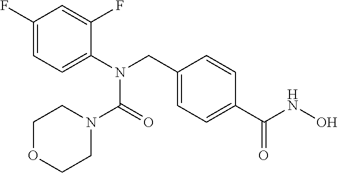 [SMILES: O=C(NO)c1ccc(CN(C(=O)N2CCOCC2)c2ccc(F)cc2F)cc1] |
| 414 | 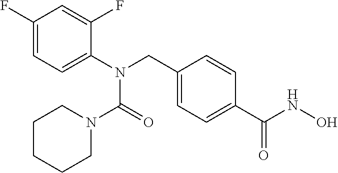 [SMILES: O=C(NO)c1ccc(CN(C(=O)N2CCCCC2)c2ccc(F)cc2F)cc1] |

TABLE 6

| Com- | |
|---|---|
| pound | Structural formula |
| 415 | 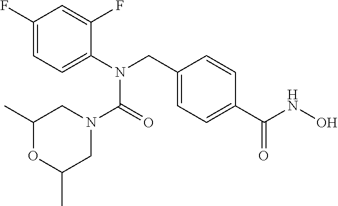 [SMILES: CC1CN(C(=O)N(Cc2ccc(C(=O)NO)cc2)c2ccc(F)cc2F)CC(C)O1] |
| 416 | 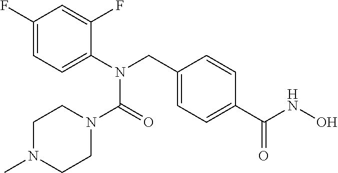 [SMILES: CN1CCN(C(=O)N(Cc2ccc(C(=O)NO)cc2)c2ccc(F)cc2F)CC1] |
| 417 | 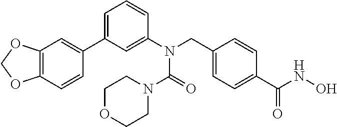 [SMILES: O=C(NO)c1ccc(CN(C(=O)N2CCOCC2)c2cccc(-c3ccc4c(c3)OCO4)c2)cc1] |
| 418 | 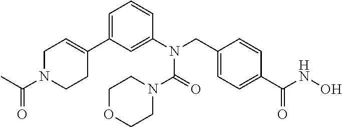 [SMILES: CC(=O)N1CC=C(c2cccc(N(Cc3ccc(C(=O)NO)cc3)C(=O)N3CCOCC3)c2)CC1] |
| 419 | 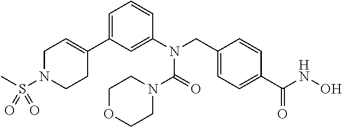 [SMILES: CS(=O)(=O)N1CC=C(c2cccc(N(Cc3ccc(C(=O)NO)cc3)C(=O)N3CCOCC3)c2)CC1] |
| 420 | 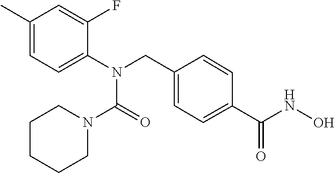 [SMILES: Cc1ccc(N(Cc2ccc(C(=O)NO)cc2)C(=O)N2CCCCC2)c(F)c1] |
| 438 | 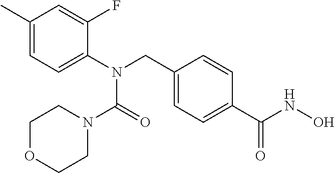 [SMILES: Cc1ccc(N(Cc2ccc(C(=O)NO)cc2)C(=O)N2CCOCC2)c(F)c1] |
| 439 | 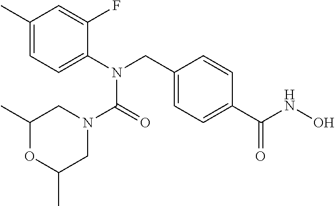 [SMILES: Cc1ccc(N(Cc2ccc(C(=O)NO)cc2)C(=O)N2CC(C)OC(C)C2)c(F)c1] |
| 440 | 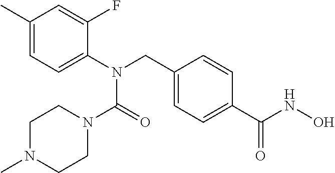 [SMILES: Cc1ccc(N(Cc2ccc(C(=O)NO)cc2)C(=O)N2CCN(C)CC2)c(F)c1] |
| 441 | 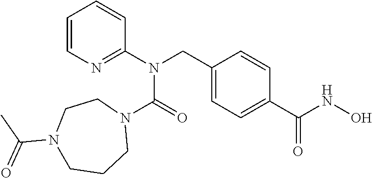 [SMILES: CC(=O)N1CCCN(C(=O)N(Cc2ccc(C(=O)NO)cc2)c2ccccn2)CC1] |
| 450 | 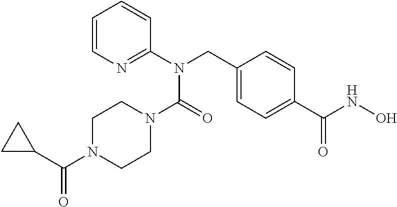 [SMILES: O=C(NO)c1ccc(CN(C(=O)N2CCN(C(=O)C3CC3)CC2)c2ccccn2)cc1] |
| 451 | 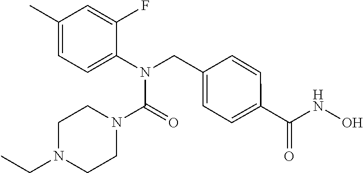 [SMILES: CCN1CCN(C(=O)N(Cc2ccc(C(=O)NO)cc2)c2ccc(C)cc2F)CC1] |
| 453 | 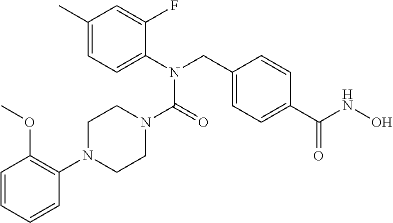 [SMILES: COc1ccccc1N1CCN(C(=O)N(Cc2ccc(C(=O)NO)cc2)c2ccc(C)cc2F)CC1] |
| 454 | 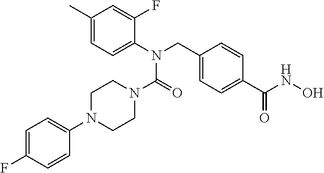 [SMILES: Cc1ccc(N(Cc2ccc(C(=O)NO)cc2)C(=O)N2CCN(c3ccc(F)cc3)CC2)c(F)c1] |
| 455 | 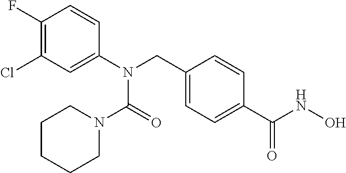 [SMILES: O=C(NO)c1ccc(CN(C(=O)N2CCCCC2)c2ccc(F)c(Cl)c2)cc1] |

TABLE 7

| Com- | |
|---|---|
| pound | Structural formula |
| 456 | 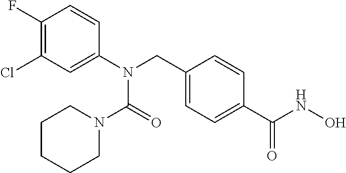 [SMILES: O=C(NO)c1ccc(CN(C(=O)N2CCCCC2)c2ccc(F)c(Cl)c2)cc1] |
| 457 | 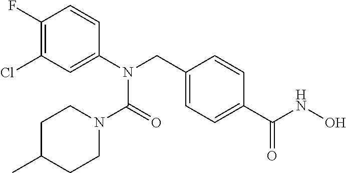 [SMILES: CC1CCN(C(=O)N(Cc2ccc(C(=O)NO)cc2)c2ccc(F)c(Cl)c2)CC1] |
| 458 | 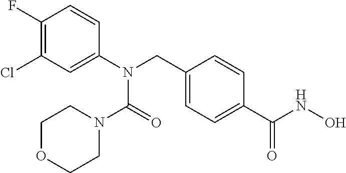 [SMILES: O=C(NO)c1ccc(CN(C(=O)N2CCOCC2)c2ccc(F)c(Cl)c2)cc1] |
| 459 | 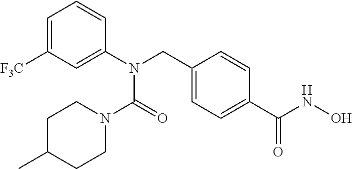 [SMILES: CC1CCN(C(=O)N(Cc2ccc(C(=O)NO)cc2)c2cccc(C(F)(F)F)c2)CC1] |
| 460 | 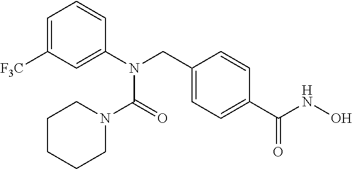 [SMILES: O=C(NO)c1ccc(CN(C(=O)N2CCCCC2)c2cccc(C(F)(F)F)c2)cc1] |
| 461 | 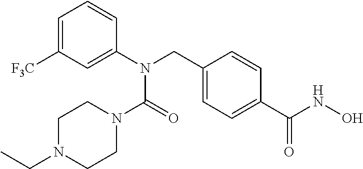 [SMILES: CCN1CCN(C(=O)N(Cc2ccc(C(=O)NO)cc2)c2cccc(C(F)(F)F)c2)CC1] |
| 462 | 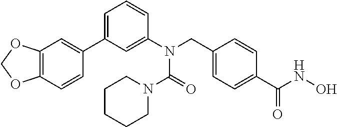 [SMILES: O=C(NO)c1ccc(CN(C(=O)N2CCCCC2)c2cccc(-c3ccc4c(c3)OCO4)c2)cc1] |
| 463 | 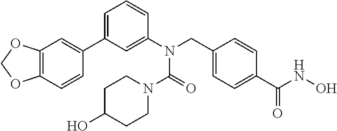 [SMILES: O=C(NO)c1ccc(CN(C(=O)N2CCC(O)CC2)c2cccc(-c3ccc4c(c3)OCO4)c2)cc1] |
| 464 | 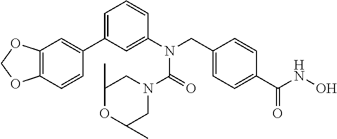 [SMILES: CC1CN(C(=O)N(Cc2ccc(C(=O)NO)cc2)c2cccc(-c3ccc4c(c3)OCO4)c2)CC(C)O1] |
| 465 | 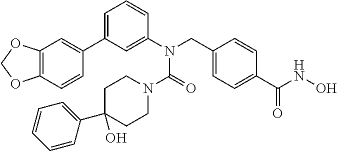 [SMILES: O=C(NO)c1ccc(CN(C(=O)N2CCC(O)(c3ccccc3)CC2)c2cccc(-c3ccc4c(c3)OCO4)c2)cc1] |
| 466 | 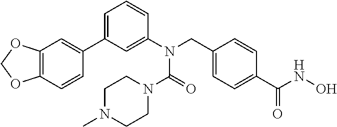 [SMILES: CN1CCN(C(=O)N(Cc2ccc(C(=O)NO)cc2)c2cccc(-c3ccc4c(c3)OCO4)c2)CC1] |
| 467 | 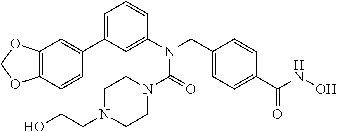 [SMILES: O=C(NO)c1ccc(CN(C(=O)N2CCN(CCO)CC2)c2cccc(-c3ccc4c(c3)OCO4)c2)cc1] |
| 468 | 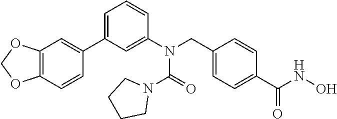 [SMILES: O=C(NO)c1ccc(CN(C(=O)N2CCCC2)c2cccc(-c3ccc4c(c3)OCO4)c2)cc1] |
| 469 | 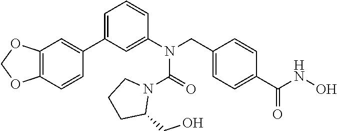 [SMILES: O=C(NO)c1ccc(CN(C(=O)N2CCC[C@H]2CO)c2cccc(-c3ccc4c(c3)OCO4)c2)cc1] |

TABLE 8

| Com- | |
|---|---|
| pound | Structural formula |
| 470 | 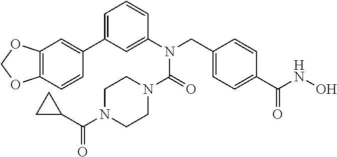 [SMILES: O=C(NO)c1ccc(CN(C(=O)N2CCN(C(=O)C3CC3)CC2)c2cccc(-c3ccc4c(c3)OCO4)c2)cc1] |
| 471 | 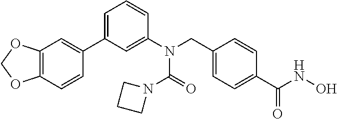 [SMILES: O=C(NO)c1ccc(CN(C(=O)N2CCC2)c2cccc(-c3ccc4c(c3)OCO4)c2)cc1] |
| 477 | 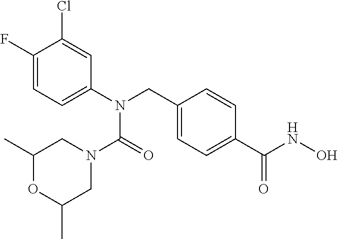 [SMILES: CC1CN(C(=O)N(Cc2ccc(C(=O)NO)cc2)c2ccc(F)c(Cl)c2)CC(C)O1] |
| 478 | 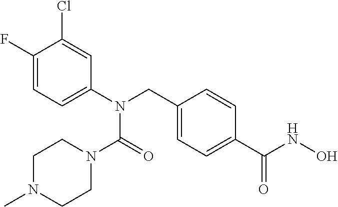 [SMILES: CN1CCN(C(=O)N(Cc2ccc(C(=O)NO)cc2)c2ccc(F)c(Cl)c2)CC1] |
| 479 | 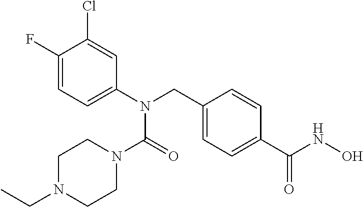 [SMILES: CCN1CCN(C(=O)N(Cc2ccc(C(=O)NO)cc2)c2ccc(F)c(Cl)c2)CC1] |
| 480 | 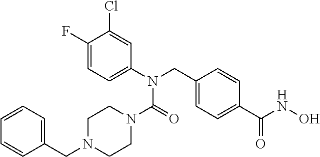 [SMILES: O=C(NO)c1ccc(CN(C(=O)N2CCN(Cc3ccccc3)CC2)c2ccc(F)c(Cl)c2)cc1] |
| 481 | 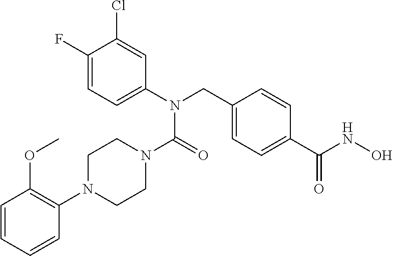 [SMILES: COc1ccccc1N1CCN(C(=O)N(Cc2ccc(C(=O)NO)cc2)c2ccc(F)c(Cl)c2)CC1] |
| 482 | 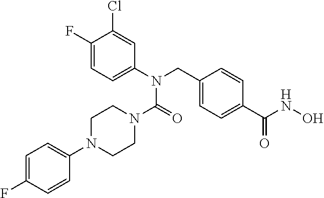 [SMILES: O=C(NO)c1ccc(CN(C(=O)N2CCN(c3ccc(F)cc3)CC2)c2ccc(F)c(Cl)c2)cc1] |
| 483 | 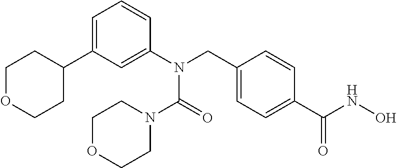 [SMILES: O=C(NO)c1ccc(CN(C(=O)N2CCOCC2)c2cccc(C3CCOCC3)c2)cc1] |
| 484 | 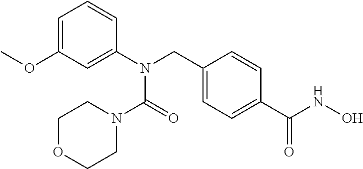 [SMILES: COc1cccc(N(Cc2ccc(C(=O)NO)cc2)C(=O)N2CCOCC2)c1] |
| 485 | 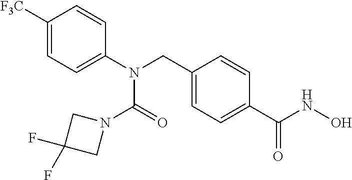 [SMILES: O=C(NO)c1ccc(CN(C(=O)N2CC(F)(F)C2)c2ccc(C(F)(F)F)cc2)cc1] |
| 486 | 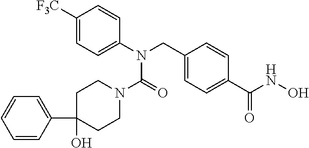 [SMILES: O=C(NO)c1ccc(CN(C(=O)N2CCC(O)(c3ccccc3)CC2)c2ccc(C(F)(F)F)cc2)cc1] |
| 487 | 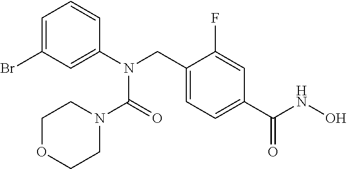 [SMILES: O=C(NO)c1ccc(CN(C(=O)N2CCOCC2)c2cccc(Br)c2)c(F)c1] |
| 488 | 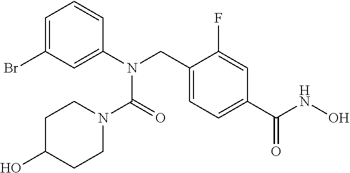 [SMILES: O=C(NO)c1ccc(CN(C(=O)N2CCC(O)CC2)c2cccc(Br)c2)c(F)c1] |

TABLE 9

| Com- | |
|---|---|
| pound | Structural formula |
| 489 | 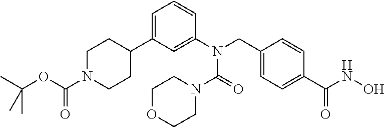 [SMILES: CC(C)(C)OC(=O)N1CCC(c2cccc(N(Cc3ccc(C(=O)NO)cc3)C(=O)N3CCOCC3)c2)CC1] |
| 490 | 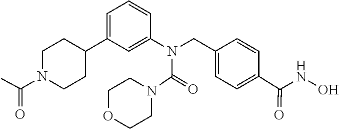 [SMILES: CC(=O)N1CCC(c2cccc(N(Cc3ccc(C(=O)NO)cc3)C(=O)N3CCOCC3)c2)CC1] |
| 491 | 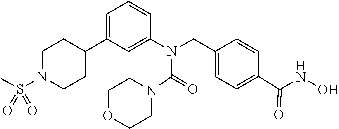 [SMILES: CS(=O)(=O)N1CCC(c2cccc(N(Cc3ccc(C(=O)NO)cc3)C(=O)N3CCOCC3)c2)CC1] |
| 492 | 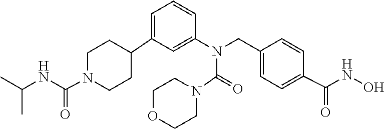 [SMILES: CC(C)NC(=O)N1CCC(c2cccc(N(Cc3ccc(C(=O)NO)cc3)C(=O)N3CCOCC3)c2)CC1] |
| 493 | 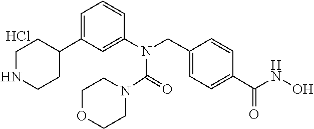 [SMILES: Cl ; O=C(NO)c1ccc(CN(C(=O)N2CCOCC2)c2cccc(C3CCNCC3)c2)cc1] |
| 494 | 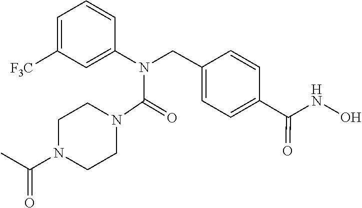 [SMILES: CC(=O)N1CCN(C(=O)N(Cc2ccc(C(=O)NO)cc2)c2cccc(C(F)(F)F)c2)CC1] |
| 495 | 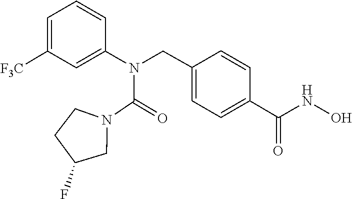 [SMILES: O=C(NO)c1ccc(CN(C(=O)N2CC[C@@H](F)C2)c2cccc(C(F)(F)F)c2)cc1] |
| 496 | 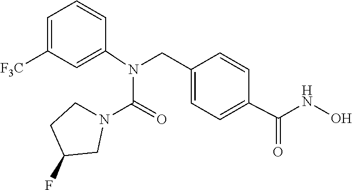 [SMILES: O=C(NO)c1ccc(CN(C(=O)N2CC[C@H](F)C2)c2cccc(C(F)(F)F)c2)cc1] |
| 497 | 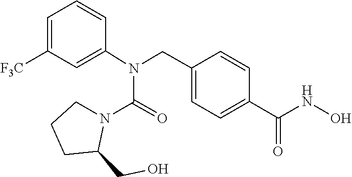 [SMILES: O=C(NO)c1ccc(CN(C(=O)N2CCC[C@@H]2CO)c2cccc(C(F)(F)F)c2)cc1] |
| 498 | 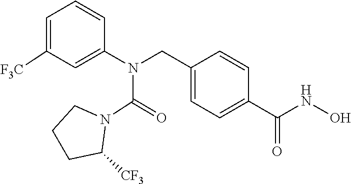 [SMILES: O=C(NO)c1ccc(CN(C(=O)N2CCC[C@H]2C(F)(F)F)c2cccc(C(F)(F)F)c2)cc1] |
| 499 | 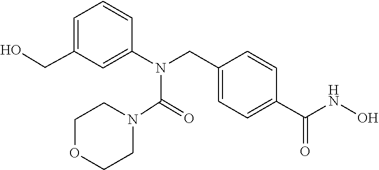 [SMILES: O=C(NO)c1ccc(CN(C(=O)N2CCOCC2)c2cccc(CO)c2)cc1] |
| 500 | 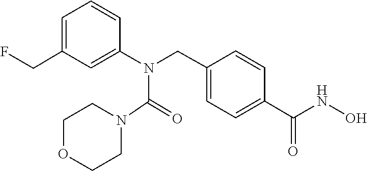 [SMILES: O=C(NO)c1ccc(CN(C(=O)N2CCOCC2)c2cccc(CF)c2)cc1] |
| 511 | 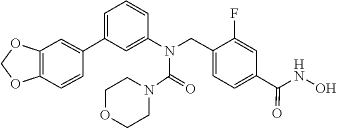 [SMILES: O=C(NO)c1ccc(CN(C(=O)N2CCOCC2)c2cccc(-c3ccc4c(c3)OCO4)c2)c(F)c1] |
| 513 | 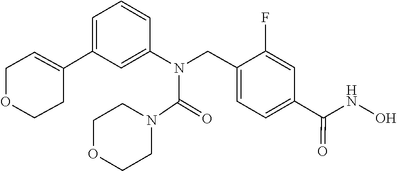 [SMILES: O=C(NO)c1ccc(CN(C(=O)N2CCOCC2)c2cccc(C3=CCOCC3)c2)c(F)c1] |

TABLE 10

| Com- | |
|---|---|
| pound | Structural formula |
| 513 | 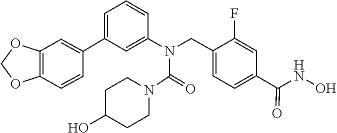 [SMILES: O=C(NO)c1ccc(CN(C(=O)N2CCC(O)CC2)c2cccc(-c3ccc4c(c3)OCO4)c2)c(F)c1] |
| 514 | 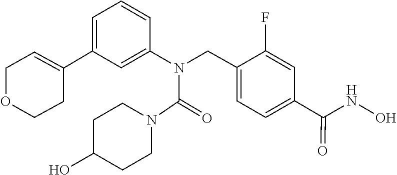 [SMILES: O=C(NO)c1ccc(CN(C(=O)N2CCC(O)CC2)c2cccc(C3=CCOCC3)c2)c(F)c1] |
| 517 | 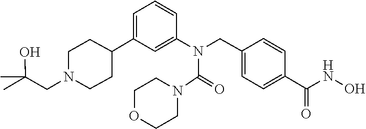 [SMILES: CC(C)(O)CN1CCC(c2cccc(N(Cc3ccc(C(=O)NO)cc3)C(=O)N3CCOCC3)c2)CC1] |
| 518 | 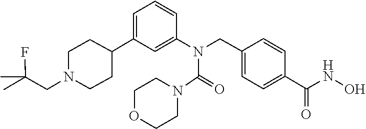 [SMILES: CC(C)(F)CN1CCC(c2cccc(N(Cc3ccc(C(=O)NO)cc3)C(=O)N3CCOCC3)c2)CC1] |
| 520 | 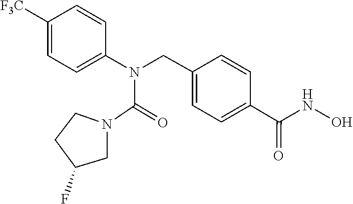 [SMILES: O=C(NO)c1ccc(CN(C(=O)N2CC[C@@H](F)C2)c2ccc(C(F)(F)F)cc2)cc1] |
| 521 | 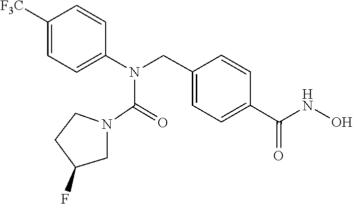 [SMILES: O=C(NO)c1ccc(CN(C(=O)N2CC[C@H](F)C2)c2ccc(C(F)(F)F)cc2)cc1] |
| 522 | 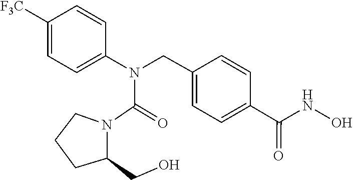 [SMILES: O=C(NO)c1ccc(CN(C(=O)N2CCC[C@@H]2CO)c2ccc(C(F)(F)F)cc2)cc1] |
| 529 | 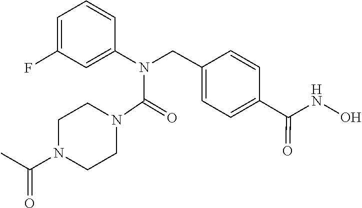 [SMILES: CC(=O)N1CCN(C(=O)N(Cc2ccc(C(=O)NO)cc2)c2cccc(F)c2)CC1] |
| 530 | 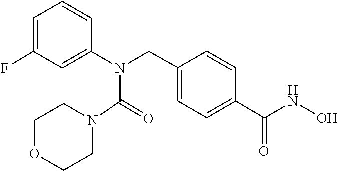 [SMILES: O=C(NO)c1ccc(CN(C(=O)N2CCOCC2)c2cccc(F)c2)cc1] |
| 531 | 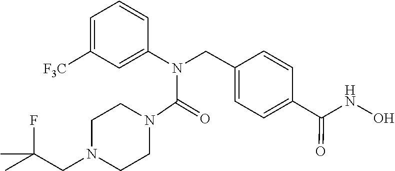 [SMILES: CC(C)(F)CN1CCN(C(=O)N(Cc2ccc(C(=O)NO)cc2)c2cccc(C(F)(F)F)c2)CC1] |
| 532 | 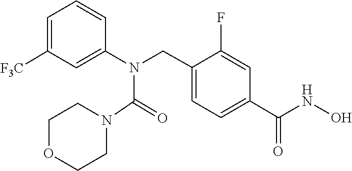 [SMILES: O=C(NO)c1ccc(CN(C(=O)N2CCOCC2)c2cccc(C(F)(F)F)c2)c(F)c1] |
| 533 | 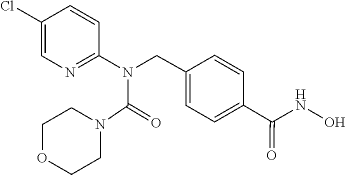 [SMILES: O=C(NO)c1ccc(CN(C(=O)N2CCOCC2)c2ccc(Cl)cn2)cc1] |
| 543 | 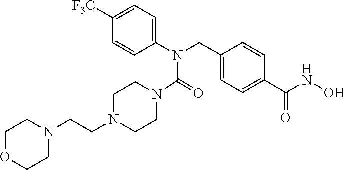 [SMILES: O=C(NO)c1ccc(CN(C(=O)N2CCN(CCN3CCOCC3)CC2)c2ccc(C(F)(F)F)cc2)cc1] |
| 544 | 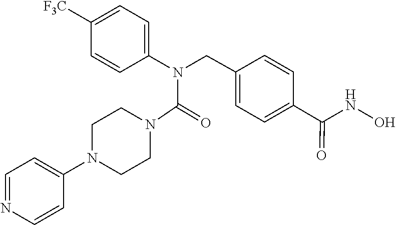 [SMILES: O=C(NO)c1ccc(CN(C(=O)N2CCN(c3ccncc3)CC2)c2ccc(C(F)(F)F)cc2)cc1] |

TABLE 11

| Com- | |
|---|---|
| pound | Structural formula |
| 545 | 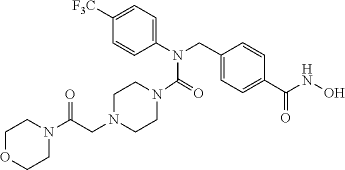 [SMILES: O=C(NO)c1ccc(CN(C(=O)N2CCN(CC(=O)N3CCOCC3)CC2)c2ccc(C(F)(F)F)cc2)cc1] |
| 577 | 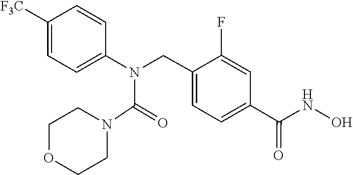 [SMILES: O=C(NO)c1ccc(CN(C(=O)N2CCOCC2)c2ccc(C(F)(F)F)cc2)c(F)c1] |
| 578 | 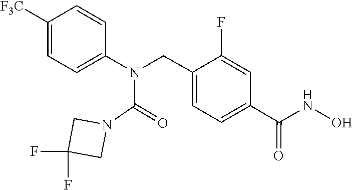 [SMILES: O=C(NO)c1ccc(CN(C(=O)N2CC(F)(F)C2)c2ccc(C(F)(F)F)cc2)c(F)c1] |
| 580 | 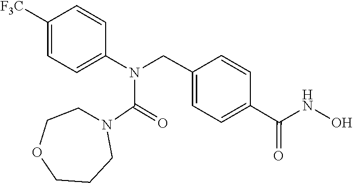 [SMILES: O=C(NO)c1ccc(CN(C(=O)N2CCCOCC2)c2ccc(C(F)(F)F)cc2)cc1] |
| 651 | 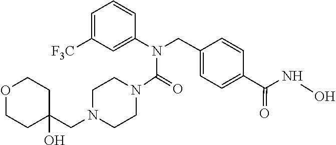 [SMILES: O=C(NO)c1ccc(CN(C(=O)N2CCN(CC3(O)CCOCC3)CC2)c2cccc(C(F)(F)F)c2)cc1] |
| 652 | 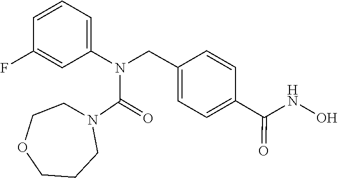 [SMILES: O=C(NO)c1ccc(CN(C(=O)N2CCCOCC2)c2cccc(F)c2)cc1] |
| 683 | 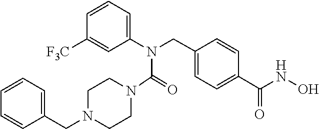 [SMILES: O=C(NO)c1ccc(CN(C(=O)N2CCN(Cc3ccccc3)CC2)c2cccc(C(F)(F)F)c2)cc1] |
| 684 | 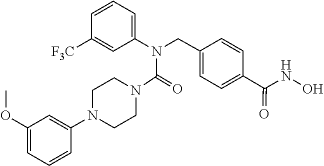 [SMILES: COc1cccc(N2CCN(C(=O)N(Cc3ccc(C(=O)NO)cc3)c3cccc(C(F)(F)F)c3)CC2)c1] |
| 716 | 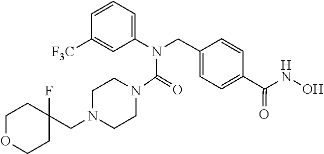 [SMILES: O=C(NO)c1ccc(CN(C(=O)N2CCN(CC3(F)CCOCC3)CC2)c2cccc(C(F)(F)F)c2)cc1] |
| 717 | 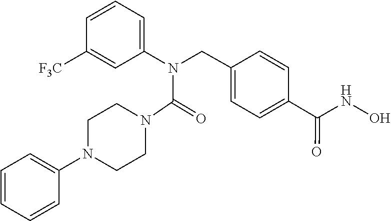 [SMILES: O=C(NO)c1ccc(CN(C(=O)N2CCN(c3ccccc3)CC2)c2cccc(C(F)(F)F)c2)cc1] |
| 718 | 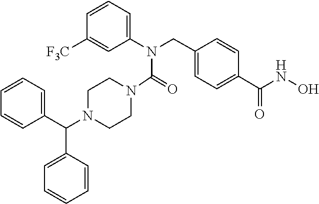 [SMILES: O=C(NO)c1ccc(CN(C(=O)N2CCN(C(c3ccccc3)c3ccccc3)CC2)c2cccc(C(F)(F)F)c2)cc1] |
| 765 | 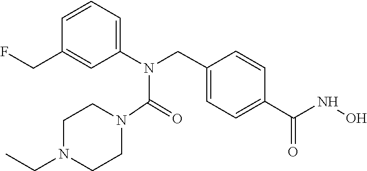 [SMILES: CCN1CCN(C(=O)N(Cc2ccc(C(=O)NO)cc2)c2cccc(CF)c2)CC1] |
| 766 | 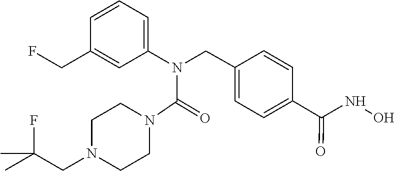 [SMILES: CC(C)(F)CN1CCN(C(=O)N(Cc2ccc(C(=O)NO)cc2)c2cccc(CF)c2)CC1] |
| 771 | 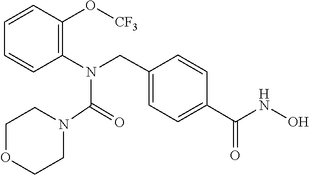 [SMILES: O=C(NO)c1ccc(CN(C(=O)N2CCOCC2)c2ccccc2OC(F)(F)F)cc1] |
| 772 | 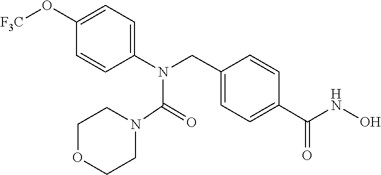 [SMILES: O=C(NO)c1ccc(CN(C(=O)N2CCOCC2)c2ccc(OC(F)(F)F)cc2)cc1] |

TABLE 12

| Com- | |
|---|---|
| pound | Structural formula |
| 773 | 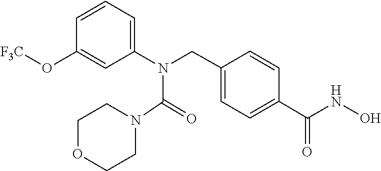 [SMILES: O=C(NO)c1ccc(CN(C(=O)N2CCOCC2)c2cccc(OC(F)(F)F)c2)cc1] |
| 774 | 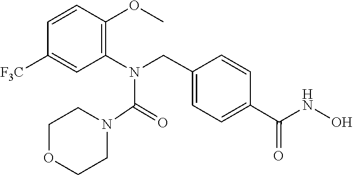 [SMILES: COc1ccc(C(F)(F)F)cc1N(Cc1ccc(C(=O)NO)cc1)C(=O)N1CCOCC1] |
| 776 | 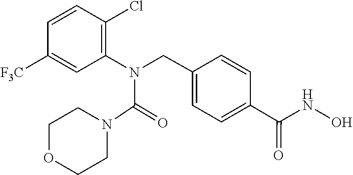 [SMILES: O=C(NO)c1ccc(CN(C(=O)N2CCOCC2)c2cc(C(F)(F)F)ccc2Cl)cc1] |
| 778 | 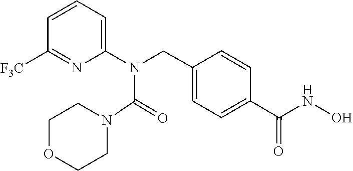 [SMILES: O=C(NO)c1ccc(CN(C(=O)N2CCOCC2)c2cccc(C(F)(F)F)n2)cc1] |
| 791 | 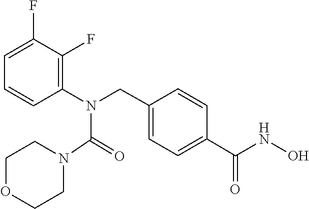 [SMILES: O=C(NO)c1ccc(CN(C(=O)N2CCOCC2)c2cccc(F)c2F)cc1] |
| 797 | 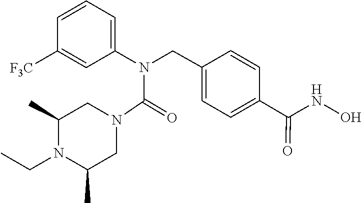 [SMILES: CCN1[C@H](C)CN(C(=O)N(Cc2ccc(C(=O)NO)cc2)c2cccc(C(F)(F)F)c2)C[C@@H]1C] |
| 800 | 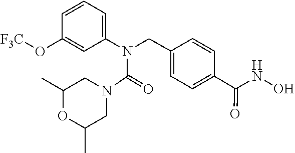 [SMILES: CC1CN(C(=O)N(Cc2ccc(C(=O)NO)cc2)c2cccc(OC(F)(F)F)c2)CC(C)O1] |
| 801 | 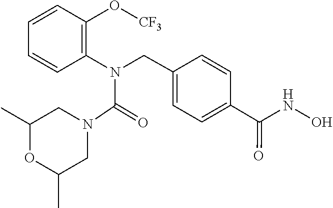 [SMILES: CC1CN(C(=O)N(Cc2ccc(C(=O)NO)cc2)c2ccccc2OC(F)(F)F)CC(C)O1] |
| 802 | 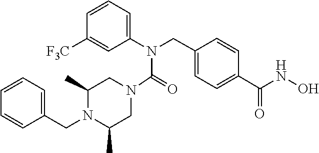 [SMILES: C[C@@H]1CN(C(=O)N(Cc2ccc(C(=O)NO)cc2)c2cccc(C(F)(F)F)c2)C[C@H](C)N1Cc1ccccc1] |
| 803 | 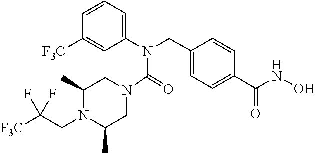 [SMILES: C[C@@H]1CN(C(=O)N(Cc2ccc(C(=O)NO)cc2)c2cccc(C(F)(F)F)c2)C[C@H](C)N1CC(F)(F)C(F)(F)F] |
| 826 | 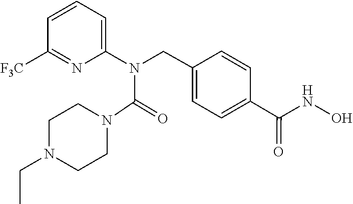 [SMILES: CCN1CCN(C(=O)N(Cc2ccc(C(=O)NO)cc2)c2cccc(C(F)(F)F)n2)CC1] |
| 827 | 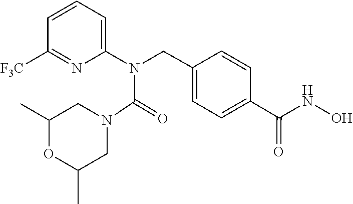 [SMILES: CC1CN(C(=O)N(Cc2ccc(C(=O)NO)cc2)c2cccc(C(F)(F)F)n2)CC(C)O1] |
| 828 | 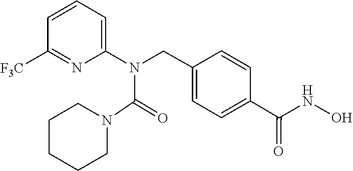 [SMILES: O=C(NO)c1ccc(CN(C(=O)N2CCCCC2)c2cccc(C(F)(F)F)n2)cc1] |
| 829 | 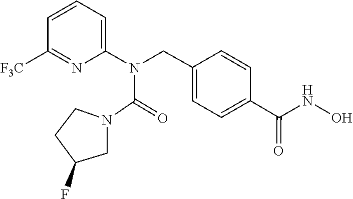 [SMILES: O=C(NO)c1ccc(CN(C(=O)N2CC[C@H](F)C2)c2cccc(C(F)(F)F)n2)cc1] |

In the present invention, the compound represented by the formula I above may be prepared by means of a method disclosed in Korea Patent Publication No. 2014-0128886 (International Patent Publication WO2014/178606), but is not limited thereto.

In the present invention, pharmaceutically acceptable salts mean the salts conventionally used in a pharmaceutical industry, and may be, for example, inorganic ion salts prepared from calcium, potassium, sodium, magnesium and the like; inorganic acid salts prepared from hydrochloric acid, nitric acid, phosphoric acid, bromic acid, iodic acid, perchloric acid, sulfuric acid and the like; organic acid salts prepared from acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carboxylic acid, vanillic acid, hydroiodic acid, etc.; sulfonic acid salts prepared from methanesulfonic acid, ethanesulfonic acid, benzenesulfonic acid, p-toluenesulfonic acid, naphthalenesulfonic acid and the like; amino acid salts prepared from glycine, arginine, lysine, etc.; amine salts prepared from trimethylamine, triethylamine, ammonia, pyridine, picoline, etc.; and the like, but types of salts meant in the present invention are not limited to those listed salts.

In the present invention, methotrexate (MTX) is a kind of antifolate, which is used as a carcinostatic agent for diseases such as leukemia, choriocarcinoma, etc. It is known that MTX irreversibly binds to dihydrofolate reductase, thus inhibiting an enzyme reaction thereof, interfering with production of tetrahydrofolates, and inhibiting various carbon transfer reactions, in which the tetrahydrofolates are involved.

In the present invention, methotrexate may be prepared by means of a method known in the art, or those products sold in the market may be used without limitation.

In the present invention, inflammatory rheumatic diseases may be at least one selected from the group consisting of rheumatoid arthritis, degenerative arthritis, reactive arthritis, enteropathic arthritis, septic arthritis, psoriatic arthritis, Reiter's syndrome, osteoarthritis, ankylosing spondylitis, Behcet's disease and lupus, and may be particularly arthritis, but not limited thereto.

In the present invention, inflammatory rheumatic diseases may be prevented or treated by means of administration of the pharmaceutical composition according to the present invention. For example, the pharmaceutical composition according to the present invention may prevent or treat inflammatory rheumatic diseases by mediating immunoregulation or inhibiting inflammations.

In embodiments of the present invention, it was identified for an adjuvant-induced arthritis model that the pharmaceutical composition containing the compound represented by the formula I above and methotrexate not only improves the conditions of arthritis such as swelling, erythema, edema, etc., and reduces an anti-CCP level, but also has an excellent effect of preventing or treating arthritis compared to a group dosed with active ingredient only.

The pharmaceutical composition of the present invention may further contain at least one type of pharmaceutically acceptable carriers for administration thereof, in addition to the compound represented by the formula I above and methotrexate. As the pharmaceutically acceptable carriers, the followings may be used: saline solution, sterilized water, Ringer's solution, buffered saline, dextrose solution, maltodextrin solution, glycerol, ethanol and a combination of at least one component thereof, and other conventional additives such as antioxidants, buffer solutions, bacteriostatic agents, etc., may be added thereto, if needed. Also, such pharmaceutical composition may be formulated into injectable dosage forms such as aqueous solutions, suspensions, emulsions, etc., pills, capsules, granules or tablets in such a way that diluents, dispersing agents, surfactants, binders and lubricants are additionally added thereto. Thus, the composition of the present invention may be patches, liquid medicines, pills, capsules, granules, tablets, suppositories, etc. These preparations may be formulated into preparations by means of a conventional method used for formulation in the art to which the present invention pertains according to each disease and/or component, or a method disclosed in Remington's Pharmaceutical Science (latest version), Mack Publishing Company, Easton Pa.

A non-limiting example of preparations for oral administration using the pharmaceutical composition of the present invention may be tablets, troches, lozenges, water soluble suspensions, oil suspensions, prepared powders, granules, emulsions, hard capsules, soft capsules, syrups, elixirs or the like. To formulate the pharmaceutical composition of the present invention into preparations for oral administration, the followings may be used: binders such as lactose, saccharose, sorbitol, mannitol, starch, amylopectin, cellulose, gelatin or the like; excipients such as dicalcium phosphate, etc.; disintegrants such as maize starch, sweet potato starch or the like; lubricants such as magnesium stearate, calcium stearate, sodium stearyl fumarate, polyethylene glycol wax or the like; etc., wherein sweetening agents, flavoring agents, syrups, etc. may also be used. Furthermore, in case of the capsules, liquid carriers such as fatty oil, etc. may be further used in addition to the above-mentioned materials.

A non-limiting example of parenteral preparations using the pharmaceutical composition of the present invention may be injectable solutions, suppositories, powders for respiratory inhalation, aerosols for spray, ointments, powders for application, oils, creams, etc. To formulate the pharmaceutical composition of the present invention into preparations for parenteral administration, the followings may be used: sterilized aqueous solutions, nonaqueous solvents, suspensions, emulsions, freeze-dried preparations, external preparations, etc. As the nonaqueous solvents and suspensions, the followings may be used, but not limited thereto: propylene glycol, polyethylene glycol, vegetable oils such as olive oil, injectable esters such as ethyl oleate, etc.

The pharmaceutical composition of the present invention may be orally or parenterally administered (for example, applied intravenously, subcutaneously, intraperitoneally or locally) according to an intended method, wherein a dosage thereof varies in a range thereof depending on a patient's weight, age, gender, health condition and diet, an administration time, an administration method, an excretion rate, a severity of a disease and the like.

A daily dosage of the compound represented by the formula I of the present invention, optical isomers thereof or pharmaceutically acceptable salts thereof may be, for example, in a range of about 0.1 to 10,000 mg/kg, in a range of about 1 to 8,000 mg/kg, in a range of about 5 to 6,000 mg/kg, or in a range of about 10 to 4,000 mg/kg, and may be preferably in a range of about 50 to 2,000 mg/kg, but not limited thereto, and may be also administered once a day or divided into several times a day. Also, a daily dosage of methotrexate of the present invention may be, for example, in a range of about 2.5 to 30 mg/kg, and may be preferably in a range of about 2.5 to 20 mg/kg, but not limited thereto, and may be also administered once a week or divided into several times a week.

A pharmaceutically effective dose and effective dosage of the pharmaceutical composition of the present invention may vary depending on a method for formulating the pharmaceutical composition, an administration mode, an administration time and/or administration route, etc., and may be diversified according to various factors including a type and degree of reactions to be achieved by means of administration of the pharmaceutical composition, a type of an individual for administration, such individual's age, weight, general health condition, disease symptom or severity, gender, diet and excretion, components of other drug compositions to be used for the corresponding individual at the same time or different times, etc., as well as other similar factors well known in a pharmaceutical field, wherein those skilled in the art may easily determine and prescribe an effective dosage for intended treatment.

The pharmaceutical composition of the present invention may be administered once a day or divided into several times a day. The pharmaceutical composition of the present invention may be administered as an individual therapeutic agent or in combination with other therapeutic agents, and may be administered sequentially or simultaneously with a conventional therapeutic agent. Considering all the factors above, the pharmaceutical composition of the present invention may be administered by an amount, in which a maximum effect may be achieved with a minimum amount without a side effect, wherein such amount may be easily determined by those skilled in the art to which the present invention pertains.

The pharmaceutical composition of the present invention may exhibit an excellent effect even when solely used, but may be further used in combination with various methods such as hormone therapy, drug treatment, etc. to increase a therapeutic efficiency.

The present invention also provides a method for preventing or treating inflammatory rheumatic diseases, including administering a therapeutically effective amount of the compound represented by the formula I above, optical isomers thereof or pharmaceutically acceptable salts thereof; and methotrexate into individuals in need.

The treatment method of the present invention may exhibit a synergy effect or an additive effect on treatment of inflammatory rheumatic diseases, in such a way that the compound of the formula I above and methotrexate are administered in combination.

In embodiments of the present invention, it was identified for an adjuvant-induced arthritis model that the conditions of arthritis such as swelling, erythema, edema, etc. are improved (Tables 16 and 17); an anti-CCP level is reduced (Table 20); and a remarkable increase in treatment effect is shown compared to a group dosed with effective components only, in such a way that the compound represented by the formula I above and methotrexate are administered in combination.

As used herein, the term “therapeutically effective amount” refers to an amount of the compound represented by the formula I above, optical isomers thereof or pharmaceutically acceptable salts thereof; and methotrexate, which are effective in preventing or treating inflammatory rheumatic diseases.

In the treatment method of the present invention, a suitable total daily amount of the compound represented by the formula I above, optical isomers thereof or pharmaceutically acceptable salts thereof; and methotrexate used may be determined within a range of correct medical decision by doctors in charge, and may be, for example, in a range of about 0.1 to 10,000 mg/kg, in a range of about 1 to 8,000 mg/kg, in a range of about 5 to 6,000 mg/kg, or in a range of about 10 to 4,000 mg/kg, and preferably the amount thereof in a range of about 50 to 2,000 mg/kg may be administered once a day or divided into several times a day. However, for the purpose of the present invention, it is preferable that a specific, therapeutically effective dose should be differently applied to each certain patient depending on various factors including a type and degree of reactions to be achieved therefrom, a specific composition including a presence of other preparations used in some cases, a patient's age, weight, general health condition, gender and diet, an administration time, an administration route, a secretion rate of the composition, a treatment period and a drug used together with the specific composition or simultaneously therewith, as well as other similar factors well known in a pharmaceutical field.

Also, in the treatment method of the present invention, the compound represented by the formula I above and methotrexate may be administered by means of the same method or different methods. Particularly, the compound represented by the formula I above may be orally administered, and methotrexate may be subcutaneously administered, but not limited thereto.

The method for preventing or treating inflammatory rheumatic diseases according to the present invention includes not only dealing with the diseases themselves before expression of their symptoms, but also inhibiting or avoiding such symptoms by administering the compound represented by the formula I above and methotrexate. In managing diseases, a preventive or therapeutic dose of a certain active component may vary depending on characteristics and severity of the diseases or conditions, and a route in which the active component is administered. A dose and a frequency thereof may vary depending on an individual patient's age, weight and reactions. A suitable dose and usage may be easily selected by those having ordinary skill in the art, naturally considering such factors. Also, the method for preventing or treating inflammatory rheumatic diseases according to the present invention may further include administering a therapeutically effective amount of additional active agents, which are helpful in preventing or treating the diseases, along with the compound represented by the formula I above and methotrexate.

The present invention also provides a use of the compound represented by the formula I above, optical isomers thereof or pharmaceutically acceptable salts thereof; and methotrexate in preparing a drug for preventing or treating inflammatory rheumatic diseases. The compound represented by the formula I above, optical isomers thereof or pharmaceutically acceptable salts thereof; and methotrexate for preparing a drug may be combined with pharmaceutically acceptable adjuvants, diluents, carriers, etc., and may be prepared into complex preparations together with other active agents, thus having a synergy action.

Matters mentioned in the pharmaceutical composition, treatment method and use of the present invention are applied the same, if not contradictory to each other.

### Advantageous Effects of Invention

A pharmaceutical composition containing a compound represented by a formula I according to the present invention, optical isomers thereof or pharmaceutically acceptable salts thereof; and methotrexate may exhibit an excellent effect of preventing or treating inflammatory rheumatic diseases, thus being widely utilized for prevention or treatment of inflammatory rheumatic diseases.

### BRIEF DESCRIPTION OF DRAWINGS

FIG. 1 is a graph of showing clinical score results of each group from 9th day to 16th day after inducing an adjuvant-induced arthritis to identify a treatment effect of a compound 374 (SM374).

FIG. 2 is a graph of showing final clinical score results of each group in an adjuvant-induced arthritis model to identify a treatment effect of a compound 374 (SM374).

FIG. 3 is a graph of showing final clinical score results of each group in an adjuvant-induced arthritis model to identify a treatment effect of compounds 374, 413, 484, 530 and 652 (SM374, SM413, SM484, SM530 and SM652).

FIG. 4 is a graph of showing final body weight results of each group in an adjuvant-induced arthritis model to identify a treatment effect of compounds 374, 413, 484, 530 and 652 (SM374, SM413, SM484, SM530 and SM652).

FIG. 5 is a result of measuring anti-CCP lgG levels in an adjuvant-induced arthritis model.

FIG. 6 shows a result of pharmacokinetic analysis on the pharmaceutical composition of the present invention.

### MODE FOR THE INVENTION

Hereinafter, the present invention will be described in more detail through preparation examples and embodiments. However, these preparation examples and embodiments are provided only for the purpose of illustrating the present invention, and thus the present invention is not limited thereto. [92]

### Preparation Example 1. Synthesis of N-(4-(hydroxycarbamoyl)benzyl)-N-(3-(trifluoromethyl)phenyl)morpholine-4-carboxamide {Compound 374 (SM374)}

### [Step 1] Synthesis of methyl 4-((3-(trifluoromethyl)phenylamino)methyl)benzoate)

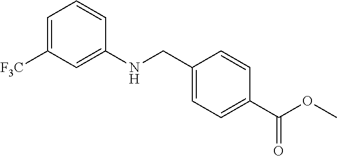 [SMILES: COC(=O)c1ccc(CNc2cccc(C(F)(F)F)c2)cc1]

3-(trifluoromethyl)benzenamine (0.30 g, 1.84 mmol) and potassium carbonate (0.76 g, 5.53 mmol) were dissolved in dimethylformamide (DMF, 5 mL), after which methyl 4-(bromomethyl)benzoate (0.42 g, 1.84 mmol) was inserted thereinto. A resulting solution was subjected to reaction at room temperature for a day and diluted with ethyl acetate. A reactant was washed with water and saturated sodium chloride aqueous solution, then dried and filtered with anhydrous magnesium sulfate, and then concentrated under reduced pressure. A residue was purified via column chromatography (silicon dioxide; ethyl acetate/hexane=20%), such that a title compound (0.37 g, 65%) was obtained.

¹H NMR (400 MHz, DMSO-d₆) δ 7.93 (d, 2H, J=8.3 Hz), 7.49 (d, 2H, J=8.3 Hz), 7.24 (t, 1H, J=7.9 Hz), 6.88-6.78 (m, 4H), 4.42 (d, 2H, J=6.1 Hz), 3.83 (s, 3H), MS (ESI) m/z 310 (M⁺+H).

### [Step 2] Synthesis of methyl 4-((((4-nitrophenoxy)carbonyl)(3-(trifluoromethyl)phenyl)amino)methyl)benzoate

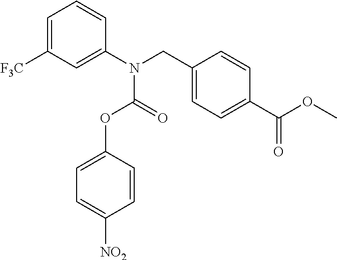 [SMILES: COC(=O)c1ccc(CN(C(=O)Oc2ccc([N+](=O)[O-])cc2)c2cccc(C(F)(F)F)c2)cc1]

Methyl 4-((3-(trifluoromethyl)phenylamino)methyl)benzoate (0.26 g, 0.82 mmol) and 4-nitrophenyl carbonochloridate (0.33 g, 1.65 mmol) were dissolved in acetonitrile (10 mL), after which potassium carbonate (0.34 g, 2.47 mmol) was inserted thereinto. A resulting solution was subjected to reaction at room temperature for a day and diluted with ethyl acetate. A reactant was washed with saturated sodium chloride aqueous solution, then dried and filtered with anhydrous sodium sulfate, and then concentrated under reduced pressure. A residue was purified via column chromatography (silicon dioxide; ethyl acetate/hexane=20%), such that a title compound (0.35 g, 89%) was obtained in a colorless oil form.

¹H NMR (400 MHz, CDCl₃) δ 8.20 (d, 2H, J=10.2 Hz), 8.01 (d, 2H, J=7.8 Hz), 7.56-7.46 (m, 3H), 7.35 (d, 3H, J=8.0 Hz), 7.26 (d, 2H, J=8.1 Hz), 5.01 (bs, 2H), 3.90 (s, 3H).

### [Step 3] Synthesis of methyl 4-((N-(3-(trifluoromethyl)phenyl)morpholine-4-carboxamido)methyl)benzoate

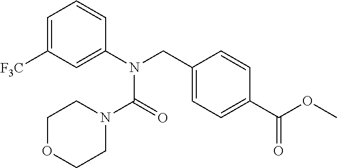 [SMILES: COC(=O)c1ccc(CN(C(=O)N2CCOCC2)c2cccc(C(F)(F)F)c2)cc1]

Methyl 4-((((4-nitrophenoxy)carbonyl)(3-(trifluoromethyl)phenyl)amino)methyl)benzoate (0.29 g, 0.60 mmol) was dissolved in dimethylformamide (10 ml), after which potassium carbonate (0.25 g, 1.81 mmol) and morpholine (0.05 mL, 0.60 mmol) were inserted thereinto. A resulting solution was subjected to reaction at 60° C. for two days, and then diluted with saturated ammonium chloride solution. Extraction was performed with ethyl acetate, after which a resulting extract was dried and filtered with anhydrous sodium sulfate, and then concentrated under reduced pressure. A residue was purified via column chromatography (silicon dioxide; ethyl acetate/hexane=50%), such that a title compound (0.15 g, 60%) was obtained.

¹H NMR (400 MHz, DMSO-d₆) δ 7.97 (d, 2H, J=8.2 Hz), 7.43-7.32 (m, 5H), 7.20 (d, 1H, J=8.0 Hz), 4.94 (s, 2H), 3.90 (s, 3H), 3.50 (t, 4H, J=4.8 Hz), 3.25 (t, 4H, J=4.8 Hz); MS (ESI) m/z 423 (M⁺+H).

### [Step 4] Synthesis of N-(4-(hydroxycarbamoyl)benzyl)-N-(3-(trifluoromethyl)phenyl)morpholine-4-carboxamide

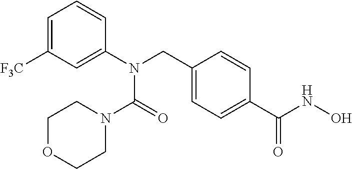 [SMILES: O=C(NO)c1ccc(CN(C(=O)N2CCOCC2)c2cccc(C(F)(F)F)c2)cc1]

Methyl 4-((N-(3-(trifluoromethyl)phenyl)morpholine-4-carboxamido)methyl)benzoate (0.15 g, 0.36 mmol) was dissolved in methanol (5 mL), after which hydroxylamine aqueous solution (50 wt %, 1 mL) and potassium hydroxide (0.10 g, 1.81 mmol) were inserted thereinto, and then stirred overnight. After a reaction was completed, a resulting solution was subjected to distillation under reduced pressure to remove methanol therefrom, after which extraction was performed with ethyl acetate and water, such that work-up was done. A resulting extract was dried and filtered with anhydrous sodium sulfate, and then concentrated under reduced pressure. A residue was stirred in diethyl ether, after which a solid product was made, filtered and dried, such that a title compound (0.082 g, 54%) was obtained in a white solid form.

¹H NMR (400 MHz, MeOD-d 3) δ 11.14 (brs, 1H), 8.99 (brs, 1H), 7.85 (d, 2H, J=8.0 Hz), 7.66-7.27 (m, 6H), 4.94 (s, 2H), 3.41 (s, 2H), 3.15 (s, 2H). MS (ESI) m/z 424 (M⁺+H).

### Preparation Example 2. Synthesis of N-(2,4-difluorophenyl)-N-(4-(hydroxycarbamoyl)benzyl)morpholine-4-carboxamide {Compound 413 (SM413)}

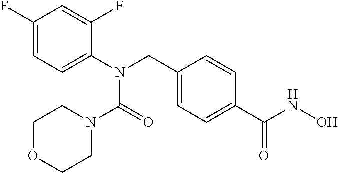 [SMILES: O=C(NO)c1ccc(CN(C(=O)N2CCOCC2)c2ccc(F)cc2F)cc1]

A title compound was obtained by means of substantially the same method as described in Example 69 of Korea Patent Publication No. 2014-0128886 (International Patent Publication WO2014/178606).

### Preparation Example 3. Synthesis of N-(4-(hydroxycarbamoyl)benzyl)-N-(3-methoxyphenyl)morpholine-4-carboxamide {Compound 484 (SM484)}

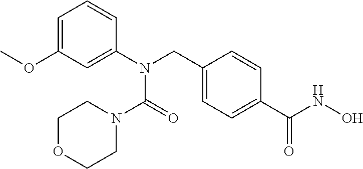 [SMILES: COc1cccc(N(Cc2ccc(C(=O)NO)cc2)C(=O)N2CCOCC2)c1]

A title compound was obtained by means of substantially the same method as described in Example 108 of Korea Patent Publication No. 2014-0128886 (International Patent Publication WO2014/178606).

### Preparation Example 4. Synthesis of N-(3-fluorophenyl)-N-(4-hydroxycarbamoyl)benzyl)morpholine-4-carboxamide {Compound 530 (SM530)}

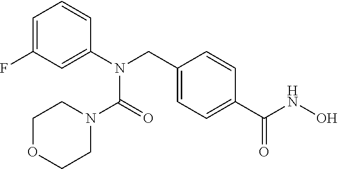 [SMILES: O=C(NO)c1ccc(CN(C(=O)N2CCOCC2)c2cccc(F)c2)cc1]

A title compound was obtained by means of substantially the same method as described in Example 135 of Korea Patent Publication No. 2014-0128886 (International Patent Publication WO2014/178606).

### Preparation Example 5. Synthesis of N-(3-fluorophenyl)-N-(4-(hydroxycarbamoyl)benzyl)-1,4-oxazepane-4-carboxamide {Compound 652 (SM652)}

### [Step 1] Synthesis of Methyl 4-(N-(3-fluorophenyl)-1,4-oxazepane-4-carboxamido)methyl)benzoate

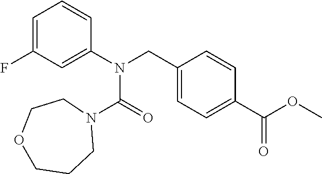 [SMILES: COC(=O)c1ccc(CN(C(=O)N2CCCOCC2)c2cccc(F)c2)cc1]

The reaction solution, in which Methyl 4-(((3-fluorophenyl)((4-nitrophenoxy)cabonyl)amino)methyl)benzoate (0.290 g, 0.683 mmol) obtained by substantially the same method as Step 2 of Preparation Example 4 described in Example 135 of Korea Patent Publication No. 2014-0128886 (International Patent Publication WO2014/178606). 1,4-oxazepane (0.188 g, 1.367 mmol) and potassium carbonate (0.283 g, 2.050 mmol) were dissolved in DMF (10 mL), was stirred at 60° C. for one day. And then, a saturated NaHCO₃ aqueous solution was poured into the reaction mixture, which was then extracted with ethyl acetate. An organic layer was washed with saturated sodium chlorate aqueous solution, then removed moisture with anhydrous magnesium sulfate, and then concentrated under reduced pressure. A concentrate was purified via column chromatography (SiO₂, 15 g cartridge; ethyl acetate/hexane=20 to 50%) and concentrated such that a title compound (0.116 g, 43.9%) was obtained in a colorless liquid form.

### [Step 2] Synthesis of N-(3-fluorophenyl)-N-(4-(hydroxycarbamoyl)benzyl)-1,4-oxazepane-4-carboxamide

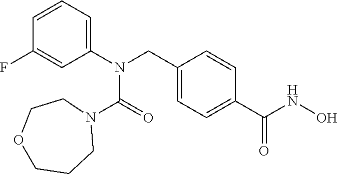 [SMILES: O=C(NO)c1ccc(CN(C(=O)N2CCCOCC2)c2cccc(F)c2)cc1]

Methyl 4-((N-(3-fluorophenyl)-1,4-oxazepane-4-carboxamido)methyl)benzoate (0.116 g, 0.3 mmol), which was a start material obtained through substantially the same method as [Step 3] of Preparation Example 1, was dissolved in methanol (10 mL), after which hydroxylamine aqueous solution (50 wt %, 1 mL) and potassium hydroxide (0.168 g, 3.01 mmol) were inserted thereinto, and then stirred overnight. After a reaction was completed, a resulting solution was subjected to distillation under reduced pressure to remove methanol therefrom, after which extraction was performed with ethyl acetate and water, such that work-up was done. A resulting extract was dried and filtered with anhydrous sodium sulfate and concentrated under reduced pressure, after which a residue was stirred in diethyl ether, such that a solid product was made, filtered and dried, and thus a title compound (0.032 g, 27.5%) was obtained in a white solid form.

### Preparation Example 6. Preparation for Adjuvant-Induced Arthritis (AIA) Model

Lewis rats (female, five-weeks old) were purchased from Central Lab Animal, Inc. and kept in a controlled environment with a temperature at 22±2° C., a humidity at 44-56% and a light/dark cycle of 12 hours, while being allowed to have a free access to standard diet and water for one week before an examination. Complete Freund's Adjuvant (Chondrex) containing 10 mg/mL of heat killed mycobacteria toxin was fully mixed together, after which 100 g thereof was taken and subcutaneously injected into each upper tail of the rats to induce arthritis therefrom.

To administer a drug of compound 374 (SM374) and evaluate an effect thereof, the rats were divided into 11 groups (9, 10 or 11 animals per group) one day before or nine days after inducing arthritis, after which each group was classified according to administered substances (vehicle, compound 374 (SM374) or methotrexate (MTX)) and administration routes (oral administration (P.O.) or intraperitoneal administration (I.P.)) as shown in a following table 13.

TABLE 13

| | Adminis- | | | Number | Adminis- |
|---|---|---|---|---|---|
| | tered dose | Drug | Dosing | of | tration |
| Group | (mg/kg) | amount | schedule | animals | route |
| Vehicle | — | — | — | 10 | P.O. |
| SM374 | 0.1 | 2 mg | Once per day | 10 | P.O. |
| | 0.5 | 10 mg | | 11 | |
| | 1 | 20 mg | | 10 | |
| | 10 | 200 mg | | 11 | |
| MTX | 1 | 4 mg | Once per week | 9 | I.P. |
| MTX + | 1 + 0.1 | 4 + 2 mg | Once per week + | 10 | I.P. + P.O. |
| SM374 | 1 + 0.5 | 4 + 10 mg | Once per day | 10 | |
| | 1 + 1 | 4 + 20 mg | | 10 | |
| | 1 + 10 | 4 + 200 mg | | 10 | |
| MTX | 3 | 24 mg | Twice per week | 10 | I.P. |

(Vehicle - Cremophor EL:ethanol:saline = 1:1:8, 5 ml/kg)

Also, in order to administer a drug of compounds 374, 413, 484, 530 and 652 (SM374, SM413, SM484, SM530 and SM652) and evaluate an effect thereof, the rats were divided into 13 groups (9 or 10 animals per group) eight days after inducing arthritis, and each group was classified according to administered substances (vehicle, SM374, SM413, SM484, SM530, SM652 and MTX) and administration routes (oral or intraperitoneal administration) as shown in a following table 14.

TABLE 14

| | Adminis- | | Number | Adminis- |
|---|---|---|---|---|
| | tered dose | Dosing | of | tration |
| Group | (mg/kg) | schedule | animals | route |
| Vehicle | | once per day | 9 | P.O. |
| SM374 | 10 | | 9 | |
| SM413 | | | 9 | |
| SM484 | | | 9 | |
| SM530 | | | 9 | |
| SM652 | | | 9 | |
| MTX | 1 | once per week | 9 | I.P. |
| MTX + SM374 | 1 + 10 | once per week + | 9 | I.P. + P.O. |
| MTX + SM413 | | once per day | 9 | |
| MTX + SM484 | | | 9 | |
| MTX + SM530 | | | 9 | |
| MTX + SM652 | | | 10 | |
| MTX | 3 | once per week | 9 | I.P. |

(Vehicle - Cremophor EL:ethanol:saline = 1:1:8, 5 ml/kg)
All the results were indicated as a mean ± standard deviation (Mean ± SD) or a mean ± standard error of the means (Mean ± SEM); statistical significance was evaluated with Mann Whitney of Graph pad prism version 4.0; and it was considered statistically significant, if p <0.05 or <0.01 in all examinations.

### Example 1: Clinical Score Analysis in AIA Model

(1) Results of Clinical Score Analysis on Compound 374

To identify the efficacy of the inventive composition on preventing or treating arthritis in an AIA model, an analysis was made on the clinical scores of rats dosed with a compound 374 (SM 374, once per day) or MTX (once per week or twice per week) from 9th day to 16th day after inducing arthritis.

Particularly, a degree of swelling, erythema, etc., in each joint of the rats were observed, after which clinical scores were calculated according to a scoring system described in a following table 15, such that the clinical scores on four joints were added up to obtain total scores for each rat (Vishwakarma et al., 2013).

TABLE 15

| Score | Clinical score |
|---|---|
| 0 | Neither swelling nor erythema appears. |
| 1 | Slight swelling and/or erythema appears. |
| 2 | Moderate or less edema appears. |
| 3 | Pronounced edema appears with a restriction on using joints. |
| 4 | Excess edema appears with rigidity of joints. |

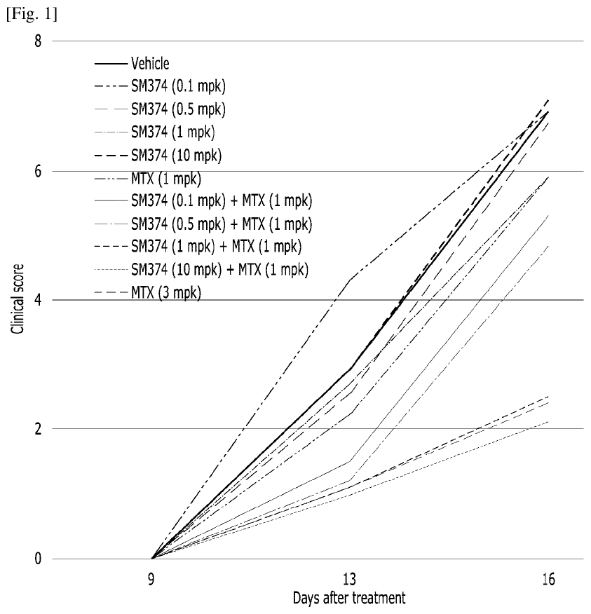

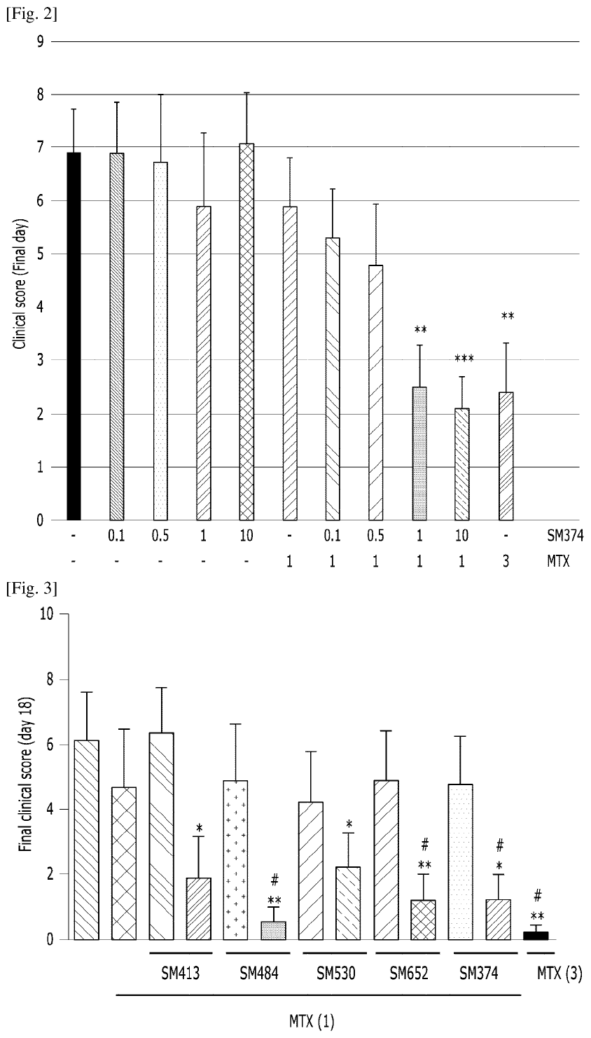

The results of clinical score analysis on compound 374 (SM374) are shown in following table 16 and FIGS. 1 and 2.

TABLE 16

| | | | | | | | Rate of |
|---|---|---|---|---|---|---|---|
| | | | Number | | | | reduction |
| | | Dosing | of | | | | in clinical |
| Group | | schedule | animals | 9th day | 13th day | 16th day | scores (%) |
| Vehicle | | — | 10 | 0.0 ± 0.0 | 2.9 ± 2.2 | 6.9 ± 2.6 | — |
| SM374 | 0.1 mg/kg | Once per | 10 | 0.0 ± 0.0 | 4.3 ± 2.9 | 6.9 ± 3.0 | 0 |
| | 0.5 mg/kg | day | 11 | 0.0 ± 0.0 | 2.6 ± 2.3 | 6.7 ± 4.2 | 2.9 |
| | 1 mg/kg | | 10 | 0.0 ± 0.0 | 2.7 ± 3.0 | 5.9 ± 4.4 | 14.5 |
| | 10 mg/kg | | 11 | 0.0 ± 0.0 | 2.9 ± 2.9 | 7.1 ± 3.1 | −2.9 |
| MTX | 1 mg/kg | Once per | 9 | 0.0 ± 0.0 | 2.2 ± 1.2 | 5.9 ± 2.8 | 14.5 |
| | | week | | | | | |
| MTX + | 1 mg/kg + | Once per | 10 | 0.0 ± 0.0 | 1.5 ± 2.0 | 5.3 ± 2.9 | 23.2 |
| SM374 | 0.1 mg/kg | week + | | | | | |
| | 1 mg/kg + | Once | 10 | 0.0 ± 0.0 | 1.2 ± 1.8 | 4.8 ± 3.6 | 30.4 |
| | 0.5 mg/kg | per day | | | | | |
| | 1 mg/kg + | | 10 | 0.0 ± 0.0 | 1.1 ± 2.2 | 2.5 ± 2.5** | 63.8 |
| | 1 mg/kg | | | | | | |
| | 1 mg/kg + | | 10 | 0.0 ± 0.0 | 1.0 ± 1.7* | 2.1 ± 1.9** | 69.6 |
| | 10 mg/kg | | | | | | |
| MTX | 3 mg/kg | Twice per | 10 | 0.0 ± 0.0 | 1.1 ± 1.6* | 2.4 ± 3.0** | 65.2 |
| | | week | | | | | |

(Mean ± SD, *P < 0.05, **P < 0.01)

As identified in FIGS. 1 and 2 and Table 16, it was identified for a group dosed with MTX and compound 374 (SM374) in combination that clinical scores are remarkably decreased compared to a group dosed with the same dosage of MTX or compound 374 (SM374) only.

The results above indicate that the administration of compound 374 (SM374) and MTX in combination shows an excellent synergy effect, and thus may be effectively used in preventing or treating arthritis.

(2) Results of Clinical Score Analysis on Compounds 374, 413, 484, 530 and 652

To identify the efficacy of the inventive composition on preventing or treating arthritis in an AIA model, an analysis was made on the clinical scores of rats dosed with compounds 374, 413, 484, 530 and 652 (SM374, SM413, SM484, SM530 and SM652, once per day) or MIX (once per week) from 8th day to 18th day after inducing arthritis. The results thereof are as shown in FIG. 3 and a table 17.

TABLE 17

| | | | | | | | Rate of |
|---|---|---|---|---|---|---|---|
| | | | | | | | reduction in |
| | Administered | Dosing | Number | | | | clinical |
| Group | dose (mg/kg) | schedule | of animals | 11th day | 15th day | 18th day | scores (%) |
| Vehicle | | — | 9 | 0.0 ± 0.0 | 1.6 ± 0.8 | 6.1 ± 1.5 | — |
| SM374 | 10 | Once per | 9 | 0.0 ± 0.0 | 1.6 ± 0.7 | 4.8 ± 1.4 | 21.3 |
| SM413 | | day | 9 | 0.0 ± 0.0 | 3.0 ± 1.4 | 6.3 ± 1.4 | −3.3 |
| SM484 | | | 9 | 0.0 ± 0.0 | 2.9 ± 1.3 | 4.9 ± 1.7 | 19.7 |
| SM530 | | | 9 | 0.0 ± 0.0 | 1.2 ± 0.5 | 4.2 ± 1.6 | 31.1 |
| SM652 | | | 9 | 0.0 ± 0.0 | 1.4 ± 0.9 | 4.9 ± 1.6 | 19.7 |
| MTX | 1 | Once per | 9 | 0.0 ± 0.0 | 1.3 ± 0.9 | 4.7 ± 1.9 | 23.0 |
| | | week | | | | | |
| MTX + | 1 ± 10 | Once per | 9 | 0.0 ± 0.0 | 0.3 ± 0.3 | 1.2 ± 0.7*, # | 80.3 |
| SM374 | | week + | | | | | |
| MTX + | | Once per | 9 | 0 0 ± 0.0 | 1.1 ± 0.8 | 1.9 ± 1.3* | 68.9 |
| SM413 | | day | | | | | |
| MTX + | | | 9 | 0.0 ± 0.0 | 0.2 ± 0.2 | 0.6 ± 0.5**, # | 90.2 |
| SM484 | | | | | | | |
| MTX + | | | 9 | 0.0 ± 0.0 | 1.4 ± 0.8 | 2.2 ± 1.0* | 63.9 |
| SM530 | | | | | | | |
| MTX + | | | 10 | 0.0 ± 0.0 | 0.5 ± 0.6 | 1.2 ± 0.9*, # | 80.3 |
| SM652 | | | | | | | |
| MTX | 3 | Once per | 9 | 0.0 ± 0.0 | 0.0 ± 0.0 | 0.2 ± 0.2**, # | 96.7 |
| | | week | | | | | |

(Mean ± SEM, [* or ** means statistical significance compared to a vehicle group, *P < 0.05, **P < 0.01]. [# means statistical significance compared to a group untreated with MTX (1 mg/kg), # P < 0.05])

As identified in FIG. 3 and Table 17, it was identified for a group dosed with compound 374, 413, 484, 530 or 652 (SM374, SM413, SM484, SM530 or SM652); and MTX in combination that clinical scores are remarkably decreased compared to a group dosed with the same dosage of compound 374, 413, 484, 530 or 652 (SM374, SM413, SM484, SM530 or SM652) or MTX only.

The results above indicate that the administration of compound 374, 413, 484, 530 or 652 (SM374, SM413, SM484, SM530 or SM652); and MIX in combination shows an excellent synergy effect, and thus may be effectively used in preventing or treating arthritis.

### Example 2: Analysis of Body Weights in ABA Model

(1) Results of Body Weight on Group Dosed with Compound 374

An analysis was made on the body weight of rats dosed with compound 374 (SM 374, once per day) or MIX (once per week or twice per week) from 9th day to 16th day after inducing AIA. The results thereof were shown in a following table 18.

TABLE 18

| | | | Number | | | |
|---|---|---|---|---|---|---|
| | | Dosing | of | Body weight (g) | | |
| Group | | schedule | animals | 9th day | 13th day | 16th day |
| Vehicle | | — | 10 | 157.4 ± 10.4 | 153.5 ± 11.9 | 147.5 ± 10.3 |
| SM374 | 0.1 mg/kg | Once per | 10 | 157.3 ± 10.1 | 151.3 ± 13.3 | 144.2 ± 10.5 |
| | 0.5 mg/kg | day | 11 | 157.3 ± 10.0 | 153.1 ± 10.8 | 148.6 ± 9.5 |
| | 1 mg/kg | | 10 | 157.3 ± 10.7 | 156.9 ± 8.9 | 151.6 ± 11.5 |
| | 10 mg/kg | | 11 | 157.3 ± 9.9 | 151.6 ± 12.2 | 147.6 ± 9.3 |
| MTX | 1 mg/kg | Once per | 9 | 157.3 ± 11.9 | 160.6 ± 4.2 | 153.8 ± 7.9 |
| | | week | | | | |
| MTX + | 1 mg/kg + | Once per | 10 | 157.2 ± 12.0 | 163.7 ± 7.0 | 154.3 ± 10.7 |
| SM374 | 0.1 mg/kg | week + | | | | |
| | 1 mg/kg + | Once per | 10 | 157.2 ± 8.4 | 157.7 ± 8.0 | 152.9 ± 12.8 |
| | 0.5 mg/kg | day | | | | |
| | 1 mg/kg + | | 10 | 157.2 ± 7.5 | 159.6 ± 8.9 | 155.0 ± 9.7 |
| | 1 mg/kg | | | | | |
| | 1 mg/kg + | | 10 | 157.2 ± 7.4 | 154.5 ± 16.0 | 154.8 ± 13.3 |
| | 10 mg/kg | | | | | |
| MTX | 3 mg/kg | Twice per | 10 | 157.2 ± 7.3 | 159.6 ± 5.7 | 157.6 ± 5.7* |
| | | week | | | | |

(Mean ± SD, an asterisk (* or **) means statistical significance compared to a vehicle group, *P < 0.05, **P < 0.01)

As shown in Table 18 above, it was identified for the group dosed with compound 374 (SM374) and MIX in combination that weights are not decreased, but significantly increased compared to the group dosed with vehicle.

(2) Results of Body Weight on Groups Dosed with Compounds 374, 413, 484, 530 and 652

An analysis was made on the body weight of rats dosed with compound 374, 413, 484, 530 or 652 (SM374, SM413, SM484, SM530 or SM652, once per day) or MIX (once per day) from 8th day to 18th day after inducing AIA.

TABLE 19

| | | | Number | | | | |
|---|---|---|---|---|---|---|---|
| | Administered | Dosing | of | Body weight (g) | | | |
| Group | dose (mg/kg) | schedule | animals | 8th day | 11th day | 15th da\y | 18th day |
| Vehicle | — | — | 9 | 151.3 ± 3.2 | 154.2 ± 2.7 | 160.3 ± 3.2 | 156.7 ± 3.7 |
| SM374 | 10 | Once per | 9 | 151.3 ± 2.1 | 153.9 ± 1.5 | 159.2 ± 1.5 | 157.6 ± 3.1 |
| SM413 | | day | 9 | 151.3 ± 2.9 | 151.4 ± 3.9 | 157.4 ± 4.2 | 155.2 ± 4.5 |
| SM484 | | | 9 | 151.4 ± 2.5 | 154.7 ± 2.1 | 160.7 ± 3.3 | 158.3 ± 4.6 |
| SM530 | | | 9 | 151.2 ± 2.6 | 153.0 ± 2.1 | 161.1 ± 2.1 | 159.2 ± 4.5 |
| SM652 | | | 9 | 152.8 ± 2.4 | 157.6 ± 1.9 | 158.8 ± 1.6 | 156.8 ± 3.9 |
| MTX | 1 | Once per | 9 | 151.2 ± 2.4 | 152.1 ± 2.3 | 156.9 ± 2.8 | 155.0 ± 3.9 |
| | | week | | | | | |
| MTX + | 1 + 10 | Once per | 9 | 151.9 ± 2.4 | 153.8 ± 1.0 | 163.3 ± 1.7 | 162.6 ± 2.8 |
| SM374 | | week + | | | | | |
| MTX + | | Once per | 9 | 151.2 ± 2.3 | 153.6 ± 2.5 | 162.3 ± 3.4 | 164.6 ± 4.5 |
| SM413 | | day | | | | | |
| MTX + | | | 9 | 151.3 ± 2.5 | 153.6 ± 2.7 | 164.0 ± 2.7 | 164.4 ± 2.5 |
| SM484 | | | | | | | |
| MTX + | | | 9 | 151.2 ± 2.3 | 151.0 ± 1.8 | 158.2 ± 2.3 | 158.1 ± 4.1 |
| SM530 | | | | | | | |
| MTX + | | | 10 | 151.3 ± 3.0 | 151.5 ± 3.4 | 159.4 ± 3.5 | 162.9 ± 3.7 |
| SM652 | | | | | | | |
| MTX | 3 | Once per | 9 | 151.2 ± 2.4 | 151.6 ± 3.8 | 160.0 ± 2.9 | 164.7 ± 2.5 |
| | | week | | | | | |

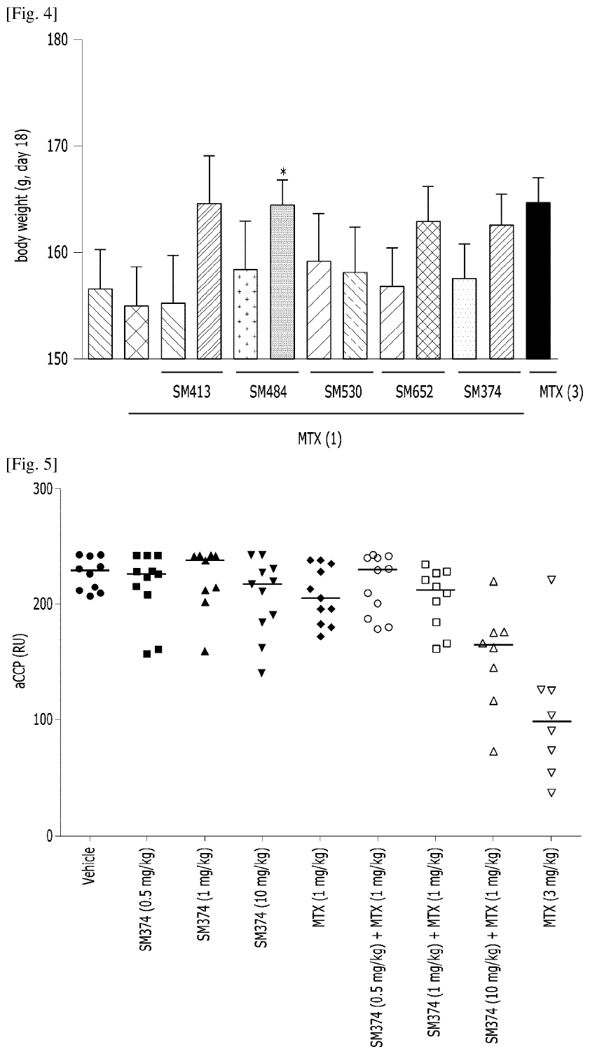

As shown in FIG. 4 and Table 19 above, it was identified for the group dosed with compound 374, 413, 484, 530 or 652 (SM374, SM413, SM484, SM530 or SM652); and MTX in combination that weights are significantly increased compared to the group dosed with vehicle.

### Example 3: Analysis of Anti-CCP IgG in AIA Model

An effect of compound 374 (SM374) and MTX on preventing or treating arthritis according to administration thereof in combination was evaluated based on analysis of anti-CCP IgG, known as a diagnosis marker for rheumatoid arthritis.

Particularly, blood was collected from each jugular vein of rats dosed with compound 374 (SM374, once per day) and MTX (once or twice per week) from 9th day to 16th day after inducing AIA, after which plasma was isolated therefrom, such that anti-CCP IgG was measured with QUANTA Lite®CCP3 (cyclic citrullinated peptide 3) IgG ELISA kit (INOVA Diagnostics Inc., U.S., catalog no. 704535) according to a protocol offered by a manufacturer thereof, wherein the results thereof are shown in FIG. 5 and Table 20.

TABLE 20

| Group | | Number of animals (N) | Anti-CCP IgG (RU) |
|---|---|---|---|
| Vehicle | | 10 | 226.2 ± 14.0 |
| SM374 | 0.5 mg/kg | 11 | 216.2 ± 30.0 |
| | 1 mg/kg | 10 | 221.8 ± 28.0 |
| | 10 mg/kg | 11 | 206.6 ± 32.9 |
| MTX | 1 mg/kg | 11 | 208.0 ± 24.3 |
| MTX + | 1 mg/kg + | 11 | 216.8 ± 25.8 |
| SM374 | 0.5 mg/kg | | |
| | 1 mg/kg + | 10 | 205.3 ± 25.9* |
| | 1 mg/kg | | |
| | 1 mg/kg + | 8 | 154.8 ± 44.1** |
| | 10 mg/kg | | |
| MTX | 3 mg/kg | 8 | 104.6 ± 56.7** |

(Mean ± SD, *P < 0.05, **P < 0.01)

As shown in FIG. 5 and Table 20, it was identified for the group dosed with compound 374 (SM374, 1 or 10 mg/kg) and MTX in combination that an anti-CCP IgG value is decreased compared to groups dosed with vehicle, MTX only and compound 374 (SM374) only.

The results above indicate that the administration of compound 374 (SM374) and MTX in combination shows an excellent synergy effect, and thus may be effectively used in preventing or treating arthritis.

### Example 4: Pharmacokinetic Analysis

A bioequivalence was evaluated through a pharmacokinetic test on administration of the inventive compound 374 (SM374) and MTX in combination as well as administration of MTX only.

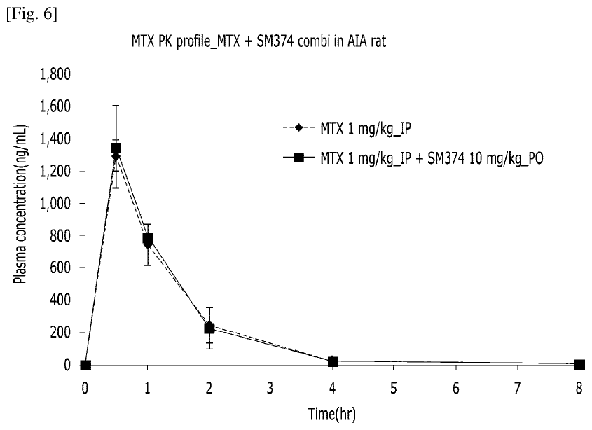

Particularly, after administration of compound 374 (SM374) and MTX, plasma was isolated from blood collected from rats for each time slot, after which organic solvent was added into such plasma to carry out deproteinization and extraction, such that a resulting sample was subjected to quantitative analysis by means of liquid chromatography and mass spectrometer (LC/MS) along with a calibration sample treated under the same conditions, to measure a concentration of compound 374 (SM374) and MTX (FIG. 6 and Table 21) in plasma of the rats as well as each PK parameter (Table 22).

TABLE 21

| | Number | Plasma concentration (ng/mL) | |
|---|---|---|---|
| | of | | MTX (1 mg/kg) + |
| Time (hr) | animals | MTX (1 mg/kg) | SM374 (10 mg/kg) |
| 0 | 3 | BLOQ | BLOQ |
| 0.5 | 3 | 1299 ± 97.3 | 1352 ± 256 |
| 1 | 3 | 747 ± 129 | 794 ± 73.5 |
| 2 | 3 | 245 ± 108 | 224 ± 128 |
| 4 | 3 | 24.4 ± 9.7 | 22.4 ± 10.2 |
| 8 | 3 | 3.0 ± 0.3 | 2.9 ± 0.2 |

TABLE 22

| | Number of | MTX | MTX (1 mg/kg) + |
|---|---|---|---|
| Time (hr) | animals | (1 mg/kg) | SM374 (10 mg/kg) |
| T1/2 (hr) | 3 | 0.9 ± 0.0 | 0.8 ± 0.0 |
| Tₘₐₓ (hr) | 3 | 0.5 ± 0.0 | 0.5 ± 0.0 |
| Cₘₐₓ (ng/mL) | 3 | 1299 ± 97.3 | 1352 ± 256 |
| AUC_{0→8h} | 3 | 1502 ± 231 | 1518 ± 139 |
| (ng · hr/mL) | | | |
| AUC_{inf} | 3 | 1506 ± 231 | 1522 ± 139 |
| (ng · hr/mL) | | | |

As shown in FIG. 6 and Tables 21 and 22, it was identified that administration of compound 374 (SM374) and MTX in combination is pharmacokinetically similar compared to administration of MTX only.

While specific portions of the present invention have been described in detail above, it is apparent to those having ordinary skill in the art that such detailed descriptions are set forth to illustrate exemplary embodiments only, but are not construed to limit the scope of the present invention. Thus, it should be understood that the substantial scope of the present invention is defined by the accompanying claims and equivalents thereto.

1. A pharmaceutical composition for preventing or treating inflammatory rheumatic diseases, comprising a histone deacetylase inhibitor and methotrexate.

2. The pharmaceutical composition according to claim 1, wherein the histone deacetylase inhibitor is a compound represented by a following formula I, optical isomers thereof or pharmaceutically acceptable salts thereof: 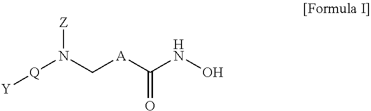
wherein
A is 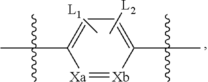
Xa and Xb are each independently CH or N,
L₁ and L₂ are each independently hydrogen, halogen, —CF₃, or —C₁₋₃ straight or branched alkyl,
Q is C(═O), S(═O)₂, S(═O) or C(═NH), and
Y is selected from a following group: 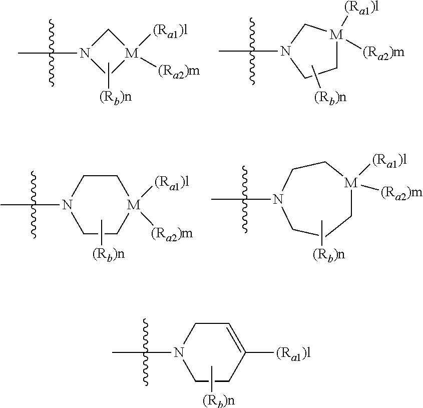
M is C, N, O, S or S(═O)₂, (here, if M is C, l and m are 1; if M is N, l is 1 and m is 0; and if M is O, S or S(═O)₂, l and m are 0),
Rₐ₁ and Rₐ₂ are each independently hydrogen; hydroxy; —C₁₋₄ straight or branched alkyl, which is unsubstituted or substituted with at least one halogen; —C₁₋₄ straight or branched alcohol; benzhydryl; —C₁₋₄ straight or branched alkyl, which is substituted with a saturated or unsaturated five to seven-membered heterocyclic compound having one to three heteroatoms out of N, O or S as a ring member (here, the heterocyclic compound may be unsubstituted or at least one hydrogen may be optionally substituted with OH, OCH₃, CH₃, CH₂CH₃ or halogen); a saturated or unsaturated five to seven-membered heterocyclic compound having one to three heteroatoms out of N, O or S as a ring member (here, the heterocyclic compound may be unsubstituted or at least one hydrogen may be optionally substituted with OH, OCH₃, CH₃, CH₂CH₃ or halogen); phenyl, wherein it is unsubstituted or at least one hydrogen is substituted with halogen, C₁₋₄ alkoxy, C₁₋₂ alkyl or hydroxy; benzyl, wherein it is unsubstituted or at least one hydrogen is substituted with halogen, C₁₋₄ alkoxy, C₁₋₂ alkyl or hydroxy; —S(═O)₂CH₃; halogen; —C₁₋₆ straight or branched alkoxy; —C₂₋₆ alkoxyalkyl; —C(═O)R_{X}, wherein R_{X} is C₁₋₃ straight or branched alkyl or C₃₋₁₀ cycloalkyl; 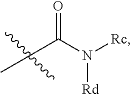
wherein R_{c} and R_{d} are independently hydrogen or C₁₋₃ straight or branched alkyl; 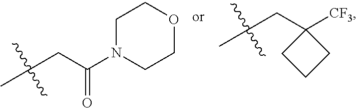 [SMILES: *CC(=O)N1CCOCC1 ; *CC1(C(F)(F)F)CCC1]
n is an integer of 0, 1 or 2,
R_{b} is hydrogen; hydroxy; —C₁₋₆ straight or branched chain alkyl, wherein it is unsubstituted or at least one hydrogen is substituted with halogen; —C(═O)CH₃; —C₁₋₄ straight or branched chain hydroxyalkyl; —C₁₋₆ straight or branched chain alkoxy; —C₂₋₆ straight or branched chain alkoxyalkyl; —CF₃; halogen; or 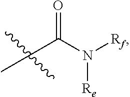
Rₑ and R_{f} are each independently hydrogen or —C₁₋₃ straight or branched chain alkyl, and
Z is selected from a following group: 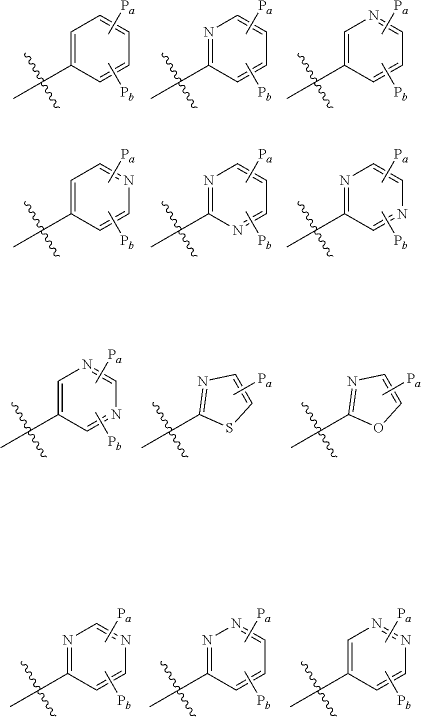
Pₐ and P_{b} are each independently 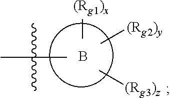
hydrogen; hydroxy; —C₁₋₄ straight or branched chain alkyl, wherein it is unsubstituted or at least one hydrogen is substituted with halogen; halogen; —CF₃; —OCF₃; —CN; —C₁₋₆ straight or branched chain alkoxy; —C₂₋₆ straight or branched chain alkyl alkoxy; —CH₂F; or —C₁₋₃ alcohol,
here 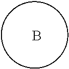
is a ring selected from phenyl, pyridine, pyrimidine, thiazole, indole, indazole, piperazine, quinoline, furan, tetrahydropyridine, piperidine or a following group: 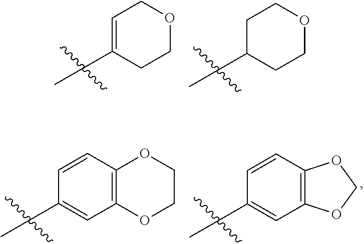 [SMILES: *C1=CCOCC1 ; *C1CCOCC1 ; *c1ccc2c(c1)OCCO2 ; *c1ccc2c(c1)OCO2]
x, y and z are each independently an integer of 0 or 1, and
R_{g1}, R_{g2} and R_{g3} are each independently selected from hydrogen; hydroxyl; —C₁₋₃ alkyl; —CF₃; —C₁₋₆ straight or branched chain alkoxy; —C₂₋₆ straight or branched chain alkyl alkoxy; —C(═O)CH₃; —C₁₋₄ straight or branched chain hydroxyalkyl; —N(CH₃)₂; halogen; phenyl; —S((═O)₂) CH₃; or a following group:

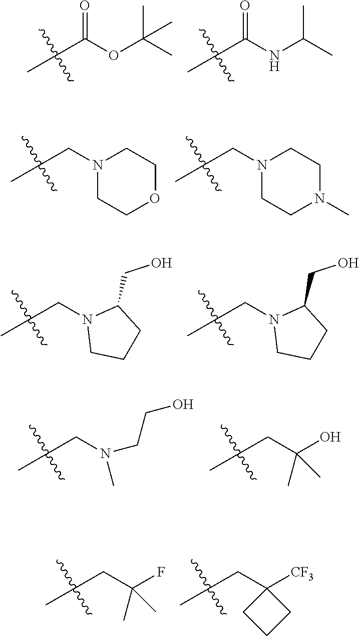

3. The pharmaceutical composition according to claim 2, wherein the histone deacetylase inhibitor is a compound represented by a following formula II, optical isomers thereof or pharmaceutically acceptable salts thereof: 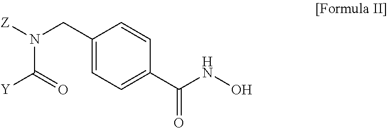
wherein
Y is selected from a following group: 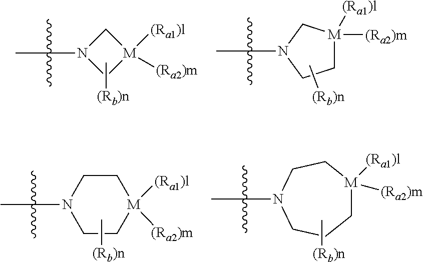
each of M, l, m, n, Rₐ₁, Rₐ₂ and R_{b} is the same with a definition of Formula I,
Z is 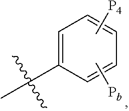
and
Pₐ and P_{b} are each independently hydrogen; hydroxy; —C₁₋₄ straight or branched chain alkyl, wherein it is unsubstituted or at least one hydrogen is substituted with halogen; halogen; —CF₃; —OCF₃; —CN; —C₁₋₆ straight or branched chain alkoxy; —C₂₋₆ straight or branched chain alkyl alkoxy; —CH₂F; or —C₁₋₃ alcohol.

4. The pharmaceutical composition according to claim 2, wherein the compound represented by the formula I above is at least one selected from the group consisting of compounds described in a following table:

| Compound | Structural formula |
|---|---|
| 252 | 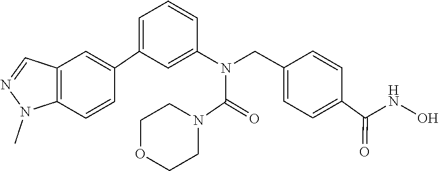 [SMILES: Cn1ncc2cc(-c3cccc(N(Cc4ccc(C(=O)NO)cc4)C(=O)N4CCOCC4)c3)ccc21] |
| 253 | 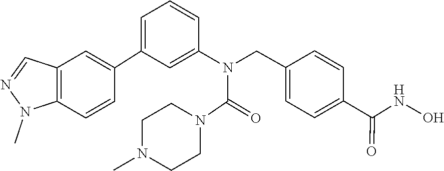 [SMILES: CN1CCN(C(=O)N(Cc2ccc(C(=O)NO)cc2)c2cccc(-c3ccc4c(cnn4C)c3)c2)CC1] |
| 254 | 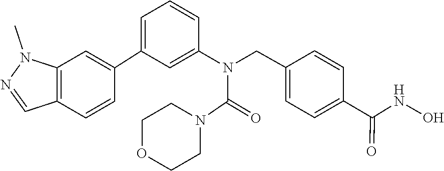 [SMILES: Cn1ncc2ccc(-c3cccc(N(Cc4ccc(C(=O)NO)cc4)C(=O)N4CCOCC4)c3)cc21] |
| 255 | 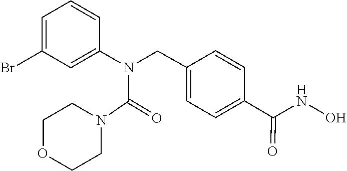 [SMILES: O=C(NO)c1ccc(CN(C(=O)N2CCOCC2)c2cccc(Br)c2)cc1] |
| 256 | 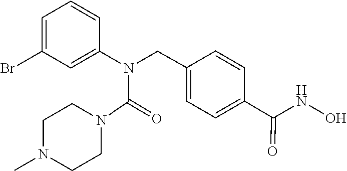 [SMILES: CN1CCN(C(=O)N(Cc2ccc(C(=O)NO)cc2)c2cccc(Br)c2)CC1] |
| 260 | 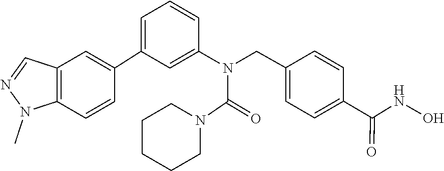 [SMILES: Cn1ncc2cc(-c3cccc(N(Cc4ccc(C(=O)NO)cc4)C(=O)N4CCCCC4)c3)ccc21] |
| 261 | 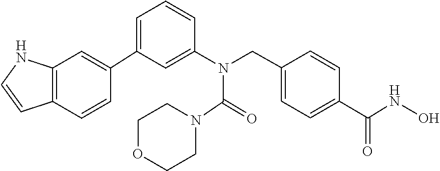 |
| 262 | 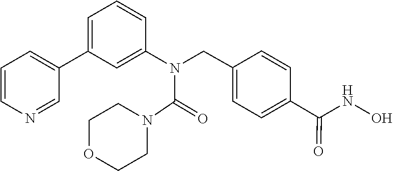 [SMILES: O=C(NO)c1ccc(CN(C(=O)N2CCOCC2)c2cccc(-c3cccnc3)c2)cc1] |
| 263 | 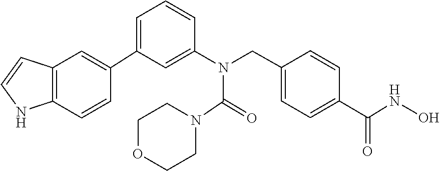 |
| 279 | 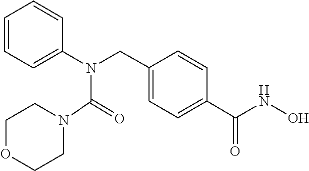 [SMILES: O=C(NO)c1ccc(CN(C(=O)N2CCOCC2)c2ccccc2)cc1] |
| 280 | 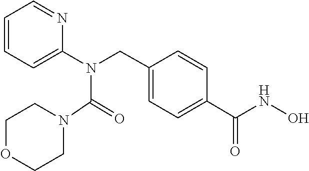 [SMILES: O=C(NO)c1ccc(CN(C(=O)N2CCOCC2)c2ccccn2)cc1] |
| 281 | 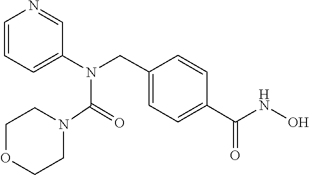 [SMILES: O=C(NO)c1ccc(CN(C(=O)N2CCOCC2)c2cccnc2)cc1] |
| 309 | 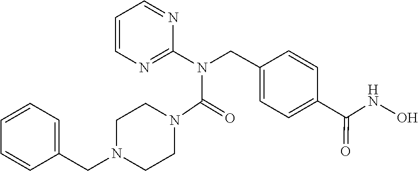 [SMILES: O=C(NO)c1ccc(CN(C(=O)N2CCN(Cc3ccccc3)CC2)c2ncccn2)cc1] |
| 311 | 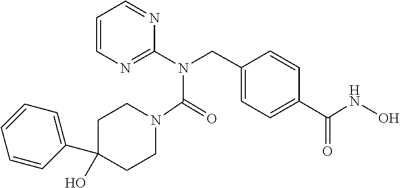 [SMILES: O=C(NO)c1ccc(CN(C(=O)N2CCC(O)(c3ccccc3)CC2)c2ncccn2)cc1] |
| 312 | 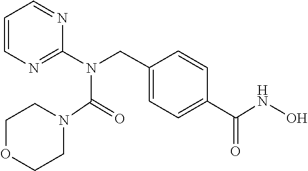 [SMILES: O=C(NO)c1ccc(CN(C(=O)N2CCOCC2)c2ncccn2)cc1] |
| 313 | 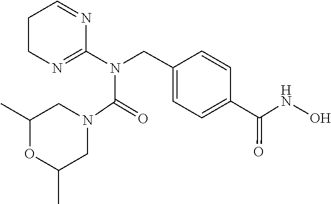 [SMILES: CC1CN(C(=O)N(Cc2ccc(C(=O)NO)cc2)C2=NCCC=N2)CC(C)O1] |
| 329 | 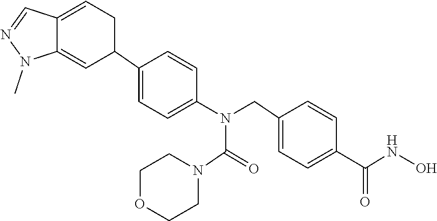 [SMILES: Cn1ncc2c1=CC(c1ccc(N(Cc3ccc(C(=O)NO)cc3)C(=O)N3CCOCC3)cc1)CC=2] |
| 330 | 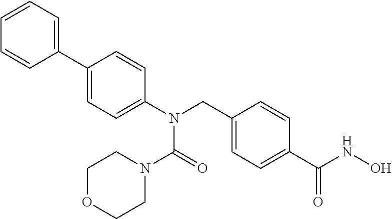 [SMILES: O=C(NO)c1ccc(CN(C(=O)N2CCOCC2)c2ccc(-c3ccccc3)cc2)cc1] |
| 331 | 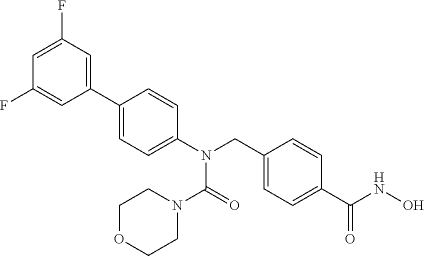 [SMILES: O=C(NO)c1ccc(CN(C(=O)N2CCOCC2)c2ccc(-c3cc(F)cc(F)c3)cc2)cc1] |
| 332 | 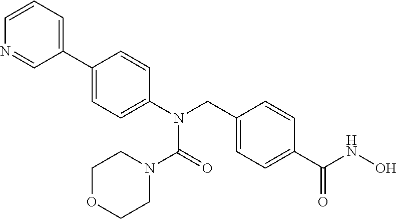 [SMILES: O=C(NO)c1ccc(CN(C(=O)N2CCOCC2)c2ccc(-c3cccnc3)cc2)cc1] |
| 333 | 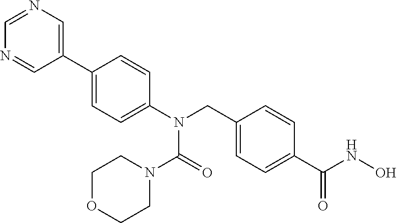 [SMILES: O=C(NO)c1ccc(CN(C(=O)N2CCOCC2)c2ccc(-c3cncnc3)cc2)cc1] |
| 334 | 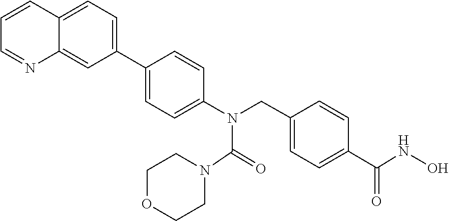 [SMILES: O=C(NO)c1ccc(CN(C(=O)N2CCOCC2)c2ccc(-c3ccc4cccnc4c3)cc2)cc1] |
| 335 | 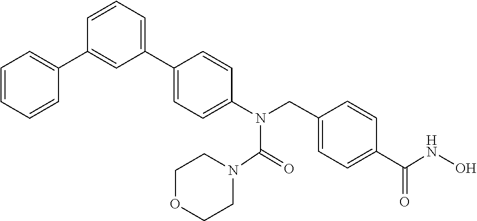 [SMILES: O=C(NO)c1ccc(CN(C(=O)N2CCOCC2)c2ccc(-c3cccc(-c4ccccc4)c3)cc2)cc1] |
| 336 | 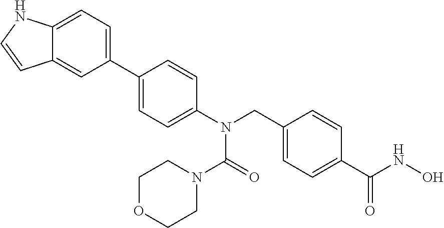 [SMILES: O=C(NO)c1ccc(CN(C(=O)N2CCOCC2)c2ccc(-c3ccc4[nH]ccc4c3)cc2)cc1] |
| 337 | 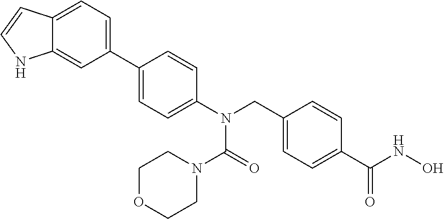 [SMILES: O=C(NO)c1ccc(CN(C(=O)N2CCOCC2)c2ccc(-c3ccc4cc[nH]c4c3)cc2)cc1] |
| 338 | 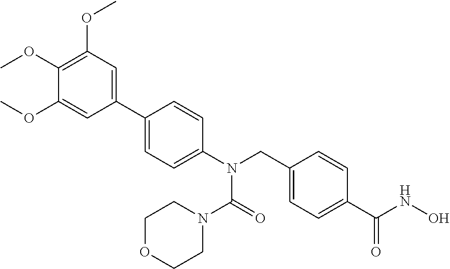 [SMILES: COc1cc(-c2ccc(N(Cc3ccc(C(=O)NO)cc3)C(=O)N3CCOCC3)cc2)cc(OC)c1OC] |
| 339 | 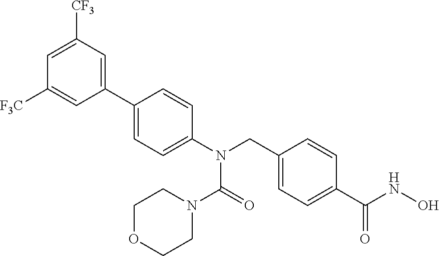 [SMILES: O=C(NO)c1ccc(CN(C(=O)N2CCOCC2)c2ccc(-c3cc(C(F)(F)F)cc(C(F)(F)F)c3)cc2)cc1] |
| 340 | 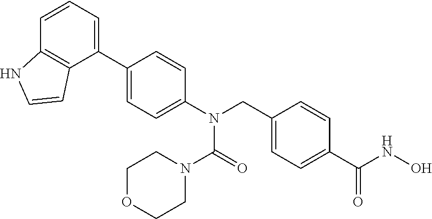 [SMILES: O=C(NO)c1ccc(CN(C(=O)N2CCOCC2)c2ccc(-c3cccc4[nH]ccc34)cc2)cc1] |
| 341 | 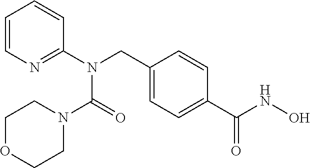 [SMILES: O=C(NO)c1ccc(CN(C(=O)N2CCOCC2)c2ccccn2)cc1] |
| 342 | 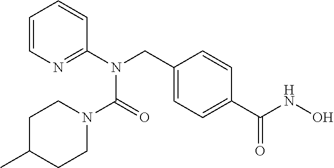 [SMILES: CC1CCN(C(=O)N(Cc2ccc(C(=O)NO)cc2)c2ccccn2)CC1] |
| 343 | 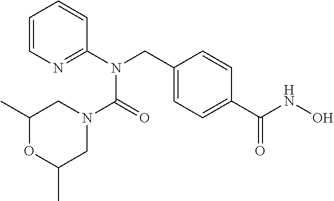 [SMILES: CC1CN(C(=O)N(Cc2ccc(C(=O)NO)cc2)c2ccccn2)CC(C)O1] |
| 352 | 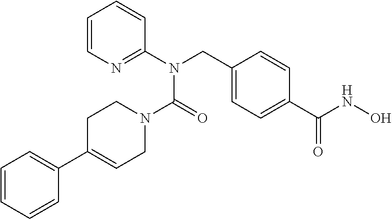 [SMILES: O=C(NO)c1ccc(CN(C(=O)N2CC=C(c3ccccc3)CC2)c2ccccn2)cc1] |
| 353 | 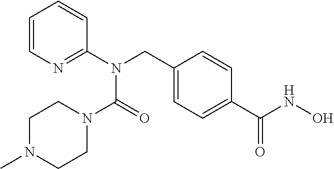 [SMILES: CN1CCN(C(=O)N(Cc2ccc(C(=O)NO)cc2)c2ccccn2)CC1] |
| 354 | 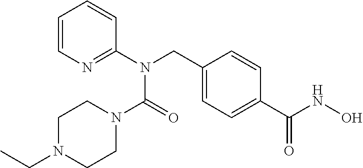 [SMILES: CCN1CCN(C(=O)N(Cc2ccc(C(=O)NO)cc2)c2ccccn2)CC1] |
| 355 | 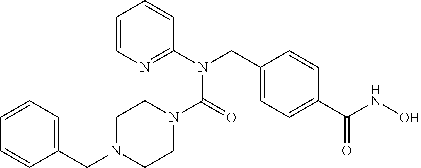 [SMILES: O=C(NO)c1ccc(CN(C(=O)N2CCN(Cc3ccccc3)CC2)c2ccccn2)cc1] |
| 356 | 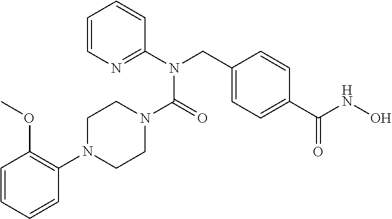 [SMILES: COc1ccccc1N1CCN(C(=O)N(Cc2ccc(C(=O)NO)cc2)c2ccccn2)CC1] |
| 357 | 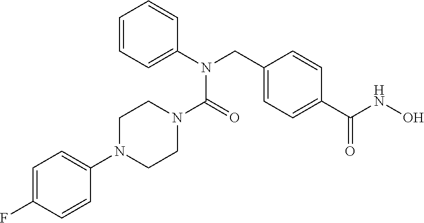 [SMILES: O=C(NO)c1ccc(CN(C(=O)N2CCN(c3ccc(F)cc3)CC2)c2ccccc2)cc1] |
| 358 | 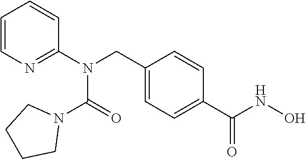 [SMILES: O=C(NO)c1ccc(CN(C(=O)N2CCCC2)c2ccccn2)cc1] |
| 370 | 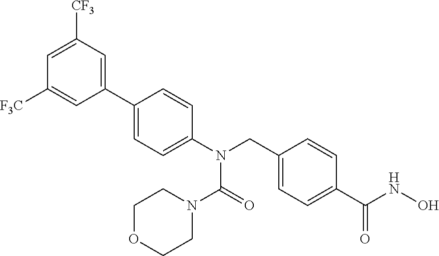 [SMILES: O=C(NO)c1ccc(CN(C(=O)N2CCOCC2)c2ccc(-c3cc(C(F)(F)F)cc(C(F)(F)F)c3)cc2)cc1] |
| 371 | 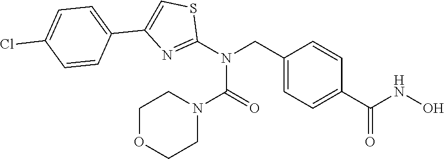 [SMILES: O=C(NO)c1ccc(CN(C(=O)N2CCOCC2)c2nc(-c3ccc(Cl)cc3)cs2)cc1] |
| 372 | 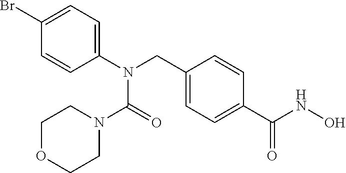 [SMILES: O=C(NO)c1ccc(CN(C(=O)N2CCOCC2)c2ccc(Br)cc2)cc1] |
| 374 | 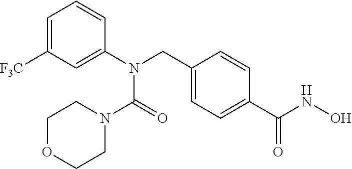 [SMILES: O=C(NO)c1ccc(CN(C(=O)N2CCOCC2)c2cccc(C(F)(F)F)c2)cc1] |
| 376 | 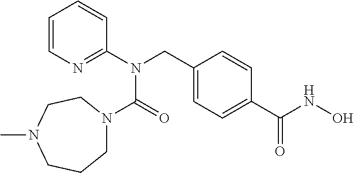 [SMILES: CN1CCCN(C(=O)N(Cc2ccc(C(=O)NO)cc2)c2ccccn2)CC1] |
| 377 | 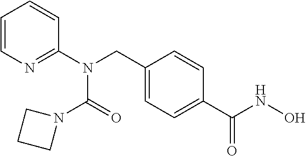 [SMILES: O=C(NO)c1ccc(CN(C(=O)N2CCC2)c2ccccn2)cc1] |
| 379 | 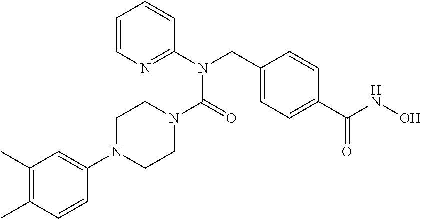 [SMILES: Cc1ccc(N2CCN(C(=O)N(Cc3ccc(C(=O)NO)cc3)c3ccccn3)CC2)cc1C] |
| 380 | 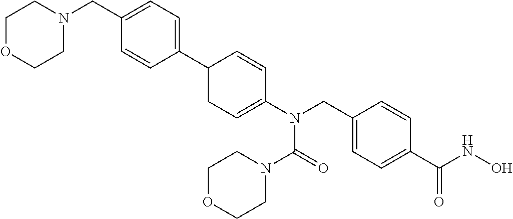 [SMILES: O=C(NO)c1ccc(CN(C(=O)N2CCOCC2)C2=CCC(c3ccc(CN4CCOCC4)cc3)C=C2)cc1] |
| 381 | 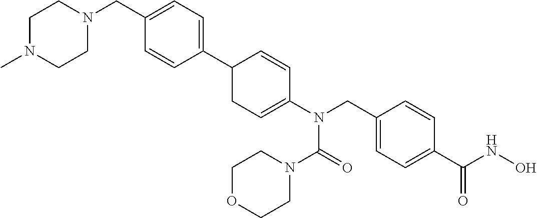 [SMILES: CN1CCN(Cc2ccc(C3C=CC(N(Cc4ccc(C(=O)NO)cc4)C(=O)N4CCOCC4)=CC3)cc2)CC1] |
| 382 | 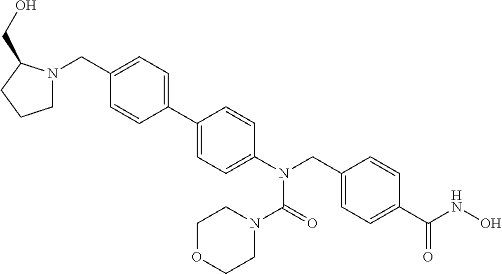 [SMILES: O=C(NO)c1ccc(CN(C(=O)N2CCOCC2)c2ccc(-c3ccc(CN4CCC[C@H]4CO)cc3)cc2)cc1] |
| 383 | 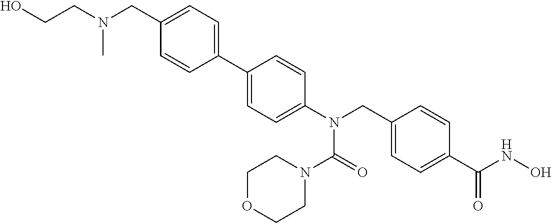 [SMILES: CN(CCO)Cc1ccc(-c2ccc(N(Cc3ccc(C(=O)NO)cc3)C(=O)N3CCOCC3)cc2)cc1] |
| 385 | 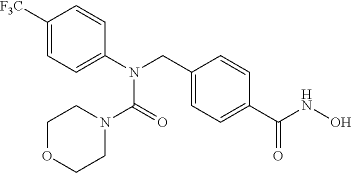 [SMILES: O=C(NO)c1ccc(CN(C(=O)N2CCOCC2)c2ccc(C(F)(F)F)cc2)cc1] |
| 386 | 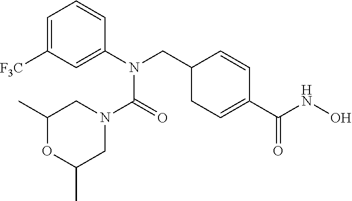 [SMILES: CC1CN(C(=O)N(CC2C=CC(C(=O)NO)=CC2)c2cccc(C(F)(F)F)c2)CC(C)O1] |
| 389 | 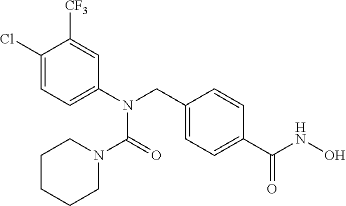 [SMILES: O=C(NO)c1ccc(CN(C(=O)N2CCCCC2)c2ccc(Cl)c(C(F)(F)F)c2)cc1] |
| 390 | 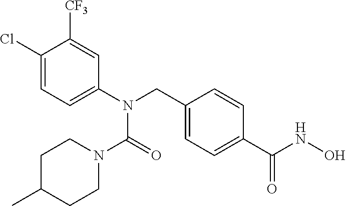 [SMILES: CC1CCN(C(=O)N(Cc2ccc(C(=O)NO)cc2)c2ccc(Cl)c(C(F)(F)F)c2)CC1] |
| 391 | 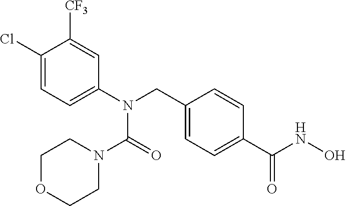 [SMILES: O=C(NO)c1ccc(CN(C(=O)N2CCOCC2)c2ccc(Cl)c(C(F)(F)F)c2)cc1] |
| 392 | 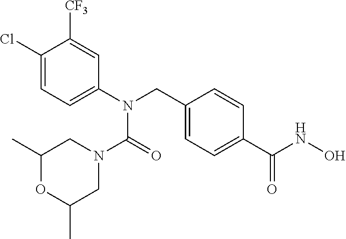 [SMILES: CC1CN(C(=O)N(Cc2ccc(C(=O)NO)cc2)c2ccc(Cl)c(C(F)(F)F)c2)CC(C)O1] |
| 393 | 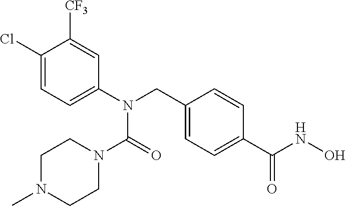 [SMILES: CN1CCN(C(=O)N(Cc2ccc(C(=O)NO)cc2)c2ccc(Cl)c(C(F)(F)F)c2)CC1] |
| 394 | 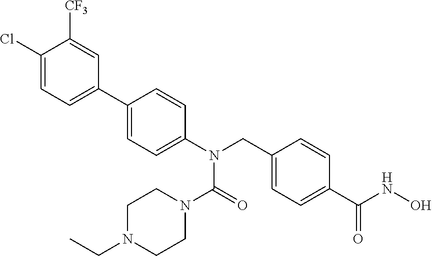 [SMILES: CCN1CCN(C(=O)N(Cc2ccc(C(=O)NO)cc2)c2ccc(-c3ccc(Cl)c(C(F)(F)F)c3)cc2)CC1] |
| 395 | 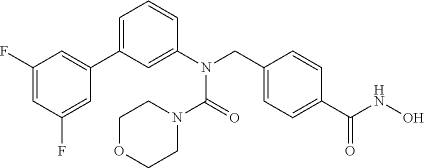 [SMILES: O=C(NO)c1ccc(CN(C(=O)N2CCOCC2)c2cccc(-c3cc(F)cc(F)c3)c2)cc1] |
| 396 | 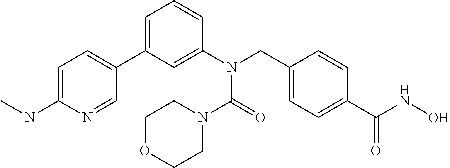 [SMILES: CNc1ccc(-c2cccc(N(Cc3ccc(C(=O)NO)cc3)C(=O)N3CCOCC3)c2)cn1] |
| 397 | 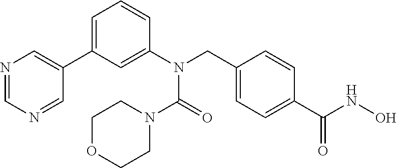 [SMILES: O=C(NO)c1ccc(CN(C(=O)N2CCOCC2)c2cccc(-c3cncnc3)c2)cc1] |
| 398 | 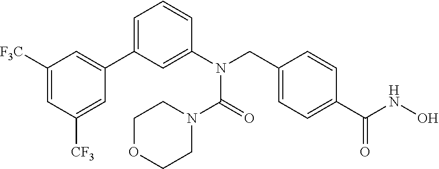 [SMILES: O=C(NO)c1ccc(CN(C(=O)N2CCOCC2)c2cccc(-c3cc(C(F)(F)F)cc(C(F)(F)F)c3)c2)cc1] |
| 399 | 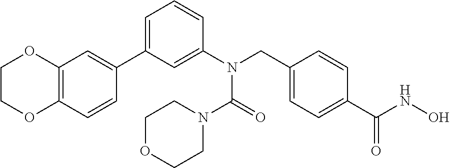 [SMILES: O=C(NO)c1ccc(CN(C(=O)N2CCOCC2)c2cccc(-c3ccc4c(c3)OCCO4)c2)cc1] |
| 400 | 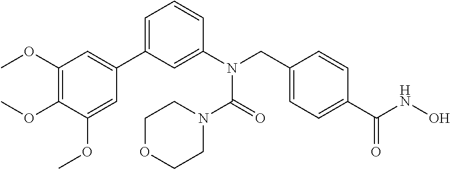 [SMILES: COc1cc(-c2cccc(N(Cc3ccc(C(=O)NO)cc3)C(=O)N3CCOCC3)c2)cc(OC)c1OC] |
| 401 | 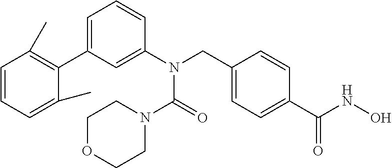 [SMILES: Cc1cccc(C)c1-c1cccc(N(Cc2ccc(C(=O)NO)cc2)C(=O)N2CCOCC2)c1] |
| 402 | 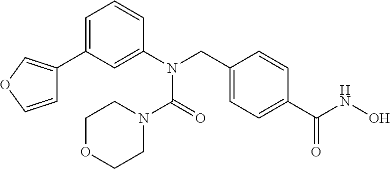 [SMILES: O=C(NO)c1ccc(CN(C(=O)N2CCOCC2)c2cccc(-c3ccoc3)c2)cc1] |
| 403 | 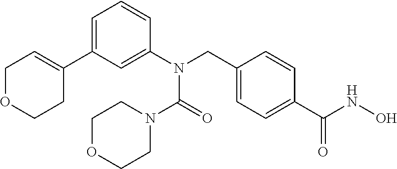 [SMILES: O=C(NO)c1ccc(CN(C(=O)N2CCOCC2)c2cccc(C3=CCOCC3)c2)cc1] |
| 404 | 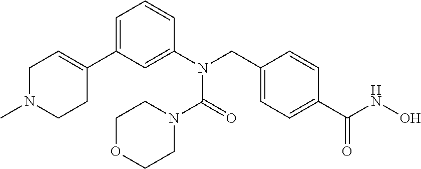 [SMILES: CN1CC=C(c2cccc(N(Cc3ccc(C(=O)NO)cc3)C(=O)N3CCOCC3)c2)CC1] |
| 405 | 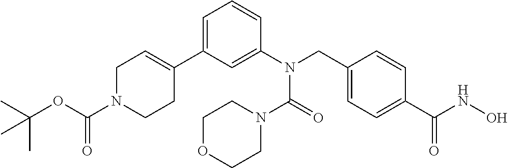 [SMILES: CC(C)(C)OC(=O)N1CC=C(c2cccc(N(Cc3ccc(C(=O)NO)cc3)C(=O)N3CCOCC3)c2)CC1] |
| 413 | 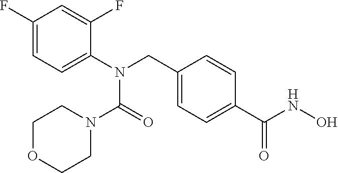 [SMILES: O=C(NO)c1ccc(CN(C(=O)N2CCOCC2)c2ccc(F)cc2F)cc1] |
| 414 | 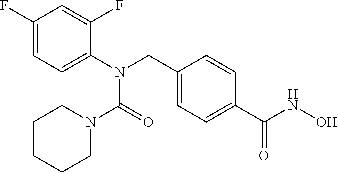 [SMILES: O=C(NO)c1ccc(CN(C(=O)N2CCCCC2)c2ccc(F)cc2F)cc1] |
| 415 | 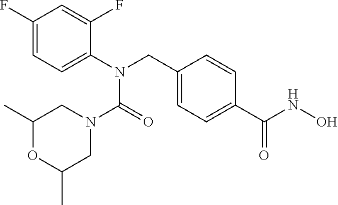 [SMILES: CC1CN(C(=O)N(Cc2ccc(C(=O)NO)cc2)c2ccc(F)cc2F)CC(C)O1] |
| 416 | 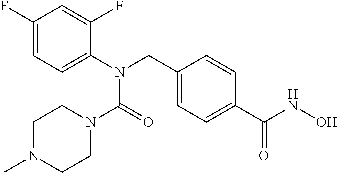 [SMILES: CN1CCN(C(=O)N(Cc2ccc(C(=O)NO)cc2)c2ccc(F)cc2F)CC1] |
| 418 | 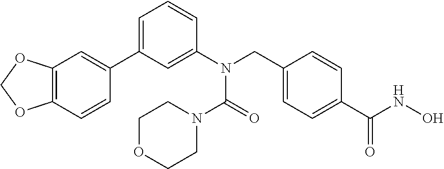 [SMILES: O=C(NO)c1ccc(CN(C(=O)N2CCOCC2)c2cccc(-c3ccc4c(c3)OCO4)c2)cc1] |
| 419 | 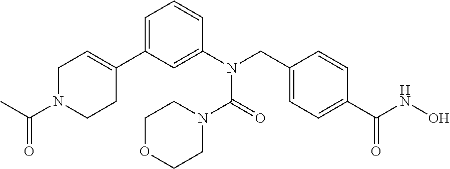 [SMILES: CC(=O)N1CC=C(c2cccc(N(Cc3ccc(C(=O)NO)cc3)C(=O)N3CCOCC3)c2)CC1] |
| 420 | 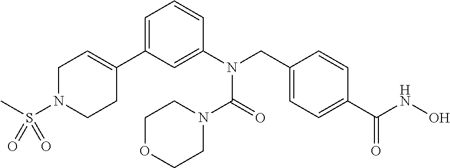 [SMILES: CS(=O)(=O)N1CC=C(c2cccc(N(Cc3ccc(C(=O)NO)cc3)C(=O)N3CCOCC3)c2)CC1] |
| 438 | 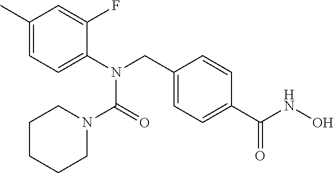 [SMILES: Cc1ccc(N(Cc2ccc(C(=O)NO)cc2)C(=O)N2CCCCC2)c(F)c1] |
| 439 | 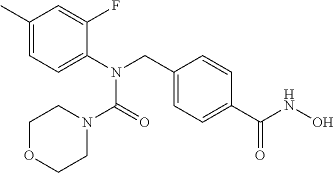 [SMILES: Cc1ccc(N(Cc2ccc(C(=O)NO)cc2)C(=O)N2CCOCC2)c(F)c1] |
| 440 | 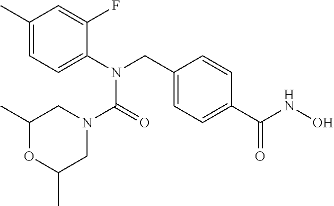 [SMILES: Cc1ccc(N(Cc2ccc(C(=O)NO)cc2)C(=O)N2CC(C)OC(C)C2)c(F)c1] |
| 441 | 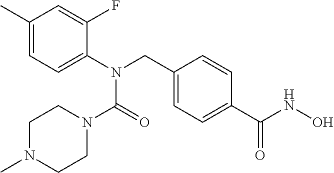 [SMILES: Cc1ccc(N(Cc2ccc(C(=O)NO)cc2)C(=O)N2CCN(C)CC2)c(F)c1] |
| 450 | 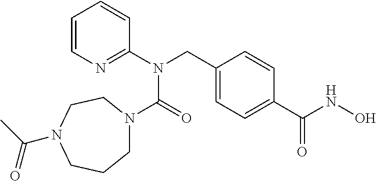 [SMILES: CC(=O)N1CCCN(C(=O)N(Cc2ccc(C(=O)NO)cc2)c2ccccn2)CC1] |
| 451 | 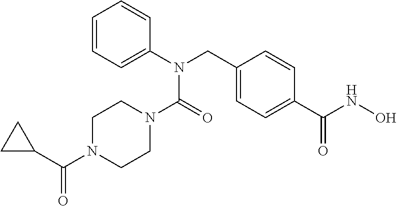 [SMILES: O=C(NO)c1ccc(CN(C(=O)N2CCN(C(=O)C3CC3)CC2)c2ccccc2)cc1] |
| 453 | 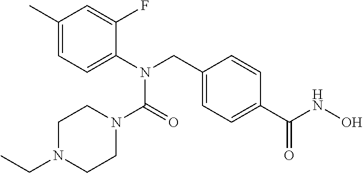 [SMILES: CCN1CCN(C(=O)N(Cc2ccc(C(=O)NO)cc2)c2ccc(C)cc2F)CC1] |
| 454 | 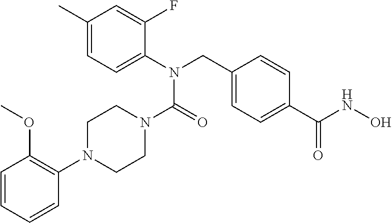 [SMILES: COc1ccccc1N1CCN(C(=O)N(Cc2ccc(C(=O)NO)cc2)c2ccc(C)cc2F)CC1] |
| 455 | 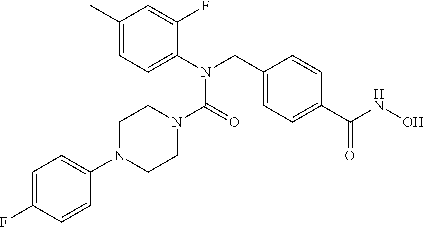 [SMILES: Cc1ccc(N(Cc2ccc(C(=O)NO)cc2)C(=O)N2CCN(c3ccc(F)cc3)CC2)c(F)c1] |
| 456 | 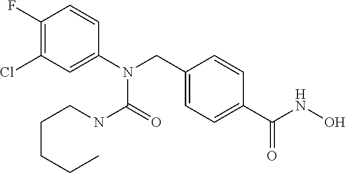 [SMILES: CCCCCNC(=O)N(Cc1ccc(C(=O)NO)cc1)c1ccc(F)c(Cl)c1] |
| 457 | 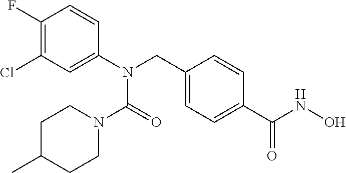 [SMILES: CC1CCN(C(=O)N(Cc2ccc(C(=O)NO)cc2)c2ccc(F)c(Cl)c2)CC1] |
| 458 | 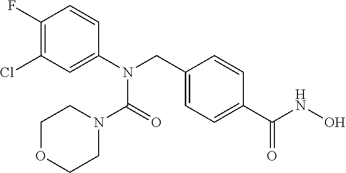 [SMILES: O=C(NO)c1ccc(CN(C(=O)N2CCOCC2)c2ccc(F)c(Cl)c2)cc1] |
| 459 | 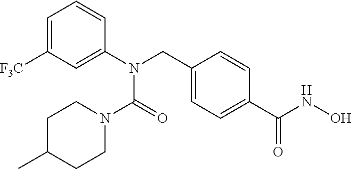 [SMILES: CC1CCN(C(=O)N(Cc2ccc(C(=O)NO)cc2)c2cccc(C(F)(F)F)c2)CC1] |
| 460 | 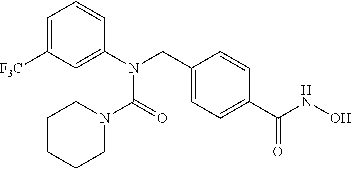 [SMILES: O=C(NO)c1ccc(CN(C(=O)N2CCCCC2)c2cccc(C(F)(F)F)c2)cc1] |
| 461 | 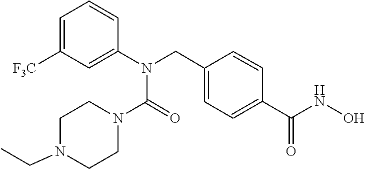 [SMILES: C=C(/C=C\C(=C/C)CN(C(=O)N1CCN(CC)CC1)c1cccc(C(F)(F)F)c1)C(=O)NO] |
| 462 | 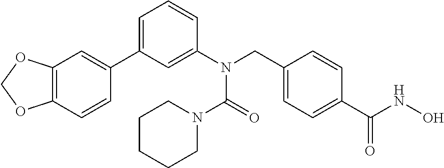 [SMILES: O=C(NO)c1ccc(CN(C(=O)N2CCCCC2)c2cccc(-c3ccc4c(c3)OCO4)c2)cc1] |
| 463 | 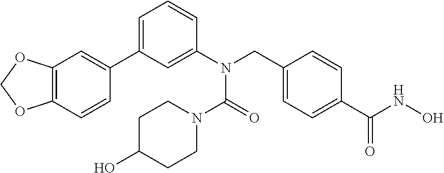 [SMILES: O=C(NO)c1ccc(CN(C(=O)N2CCC(O)CC2)c2cccc(-c3ccc4c(c3)OCO4)c2)cc1] |
| 464 | 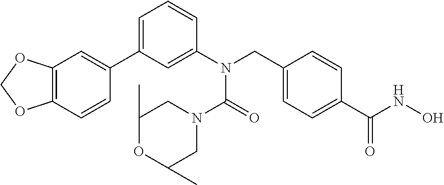 [SMILES: CC1CN(C(=O)N(Cc2ccc(C(=O)NO)cc2)c2cccc(-c3ccc4c(c3)OCO4)c2)CC(C)O1] |
| 465 | 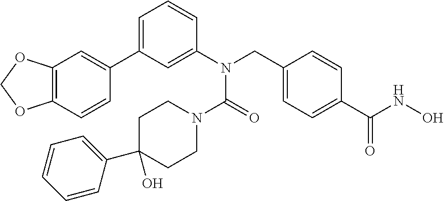 [SMILES: O=C(NO)c1ccc(CN(C(=O)N2CCC(O)(c3ccccc3)CC2)c2cccc(-c3ccc4c(c3)OCO4)c2)cc1] |
| 466 | 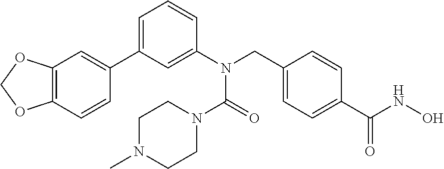 [SMILES: CN1CCN(C(=O)N(Cc2ccc(C(=O)NO)cc2)c2cccc(-c3ccc4c(c3)OCO4)c2)CC1] |
| 467 | 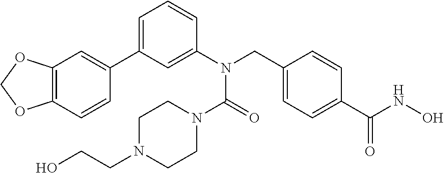 [SMILES: O=C(NO)c1ccc(CN(C(=O)N2CCN(CCO)CC2)c2cccc(-c3ccc4c(c3)OCO4)c2)cc1] |
| 468 | 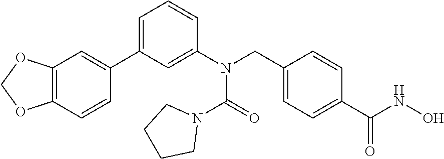 |
| 469 | 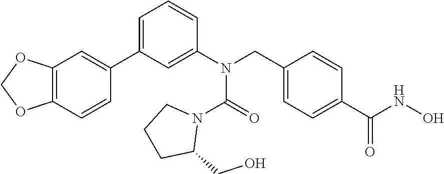 [SMILES: O=C(NO)c1ccc(CN(C(=O)N2CCC[C@H]2CO)c2cccc(-c3ccc4c(c3)OCO4)c2)cc1] |
| 470 | 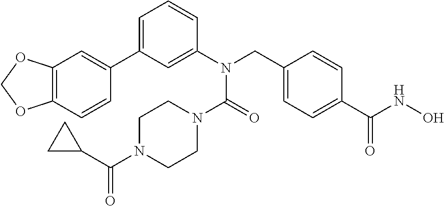 [SMILES: O=C(NO)c1ccc(CN(C(=O)N2CCN(C(=O)C3CC3)CC2)c2cccc(-c3ccc4c(c3)OCO4)c2)cc1] |
| 471 | 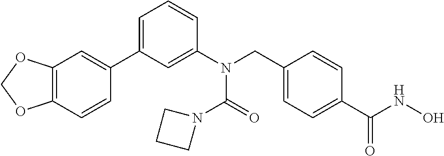 [SMILES: O=C(NO)c1ccc(CN(C(=O)N2CCC2)c2cccc(-c3ccc4c(c3)OCO4)c2)cc1] |
| 477 | 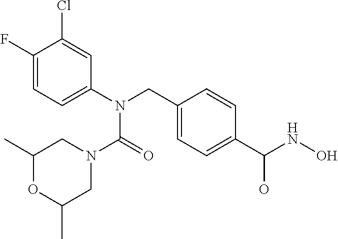 |
| 478 | 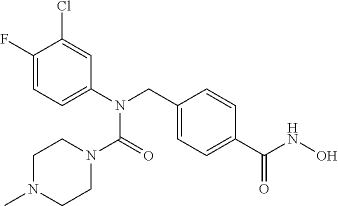 [SMILES: CN1CCN(C(=O)N(Cc2ccc(C(=O)NO)cc2)c2ccc(F)c(Cl)c2)CC1] |
| 479 | 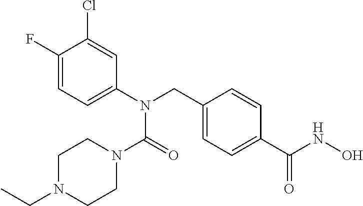 [SMILES: CCN1CCN(C(=O)N(Cc2ccc(C(=O)NO)cc2)c2ccc(F)c(Cl)c2)CC1] |
| 480 | 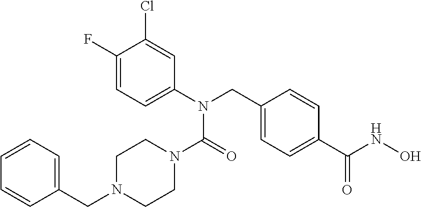 [SMILES: O=C(NO)c1ccc(CN(C(=O)N2CCN(Cc3ccccc3)CC2)c2ccc(F)c(Cl)c2)cc1] |
| 481 | 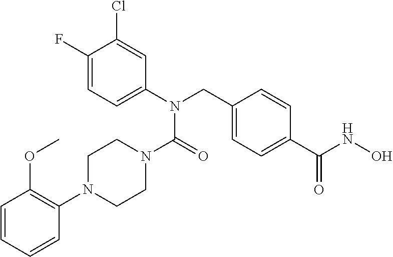 [SMILES: COc1ccccc1N1CCN(C(=O)N(Cc2ccc(C(=O)NO)cc2)c2ccc(F)c(Cl)c2)CC1] |
| 482 | 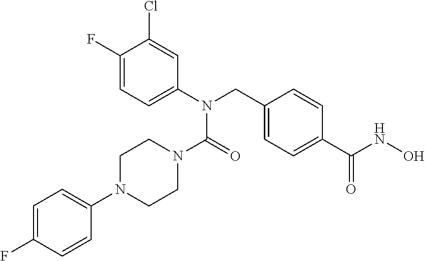 [SMILES: O=C(NO)c1ccc(CN(C(=O)N2CCN(c3ccc(F)cc3)CC2)c2ccc(F)c(Cl)c2)cc1] |
| 483 | 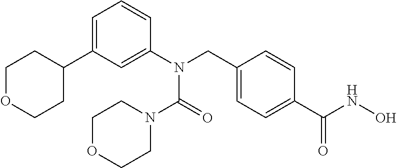 [SMILES: O=C(NO)c1ccc(CN(C(=O)N2CCOCC2)c2cccc(C3CCOCC3)c2)cc1] |
| 484 | 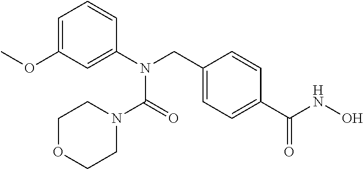 [SMILES: COc1cccc(N(Cc2ccc(C(=O)NO)cc2)C(=O)N2CCOCC2)c1] |
| 485 | 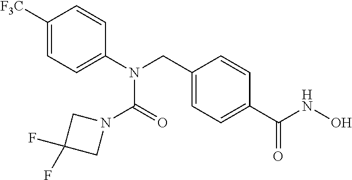 [SMILES: O=C(NO)c1ccc(CN(C(=O)N2CC(F)(F)C2)c2ccc(C(F)(F)F)cc2)cc1] |
| 486 | 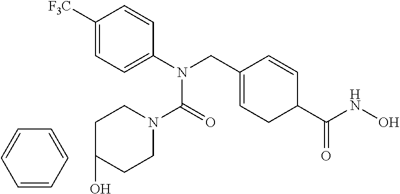 |
| 487 | 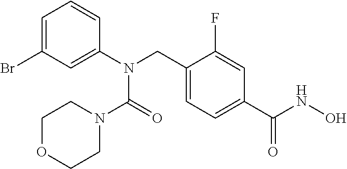 [SMILES: O=C(NO)c1ccc(CN(C(=O)N2CCOCC2)c2cccc(Br)c2)c(F)c1] |
| 488 | 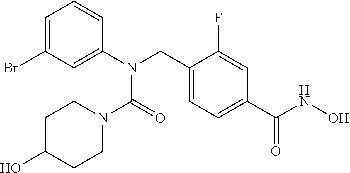 [SMILES: O=C(NO)c1ccc(CN(C(=O)N2CCC(O)CC2)c2cccc(Br)c2)c(F)c1] |
| 489 | 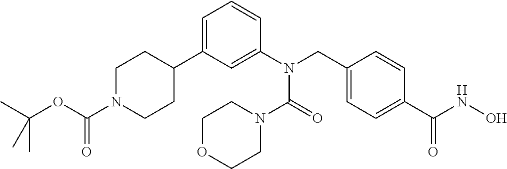 [SMILES: CC(C)(C)OC(=O)N1CCC(c2cccc(N(Cc3ccc(C(=O)NO)cc3)C(=O)N3CCOCC3)c2)CC1] |
| 490 | 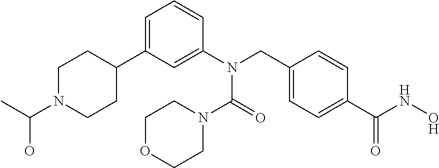 |
| 491 | 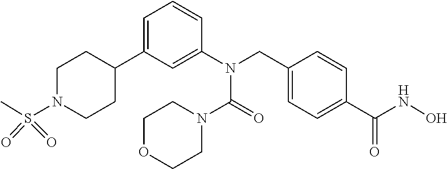 [SMILES: CS(=O)(=O)N1CCC(c2cccc(N(Cc3ccc(C(=O)NO)cc3)C(=O)N3CCOCC3)c2)CC1] |
| 492 | 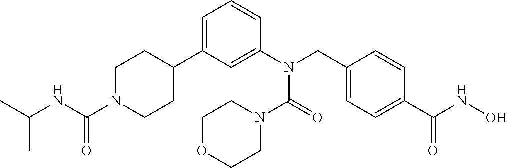 [SMILES: CC(C)NC(=O)N1CCC(c2cccc(N(Cc3ccc(C(=O)NO)cc3)C(=O)N3CCOCC3)c2)CC1] |
| 493 | 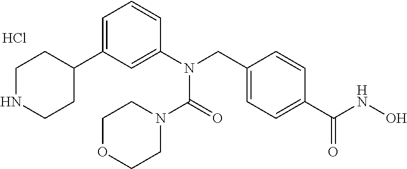 [SMILES: Cl ; O=C(NO)c1ccc(CN(C(=O)N2CCOCC2)c2cccc(C3CCNCC3)c2)cc1] |
| 494 | 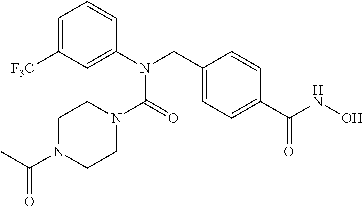 [SMILES: CC(=O)N1CCN(C(=O)N(Cc2ccc(C(=O)NO)cc2)c2cccc(C(F)(F)F)c2)CC1] |
| 495 | 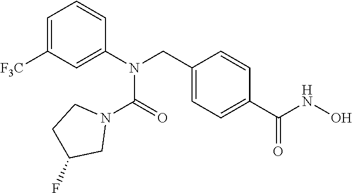 [SMILES: O=C(NO)c1ccc(CN(C(=O)N2CC[C@@H](F)C2)c2cccc(C(F)(F)F)c2)cc1] |
| 496 | 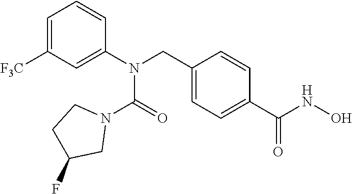 [SMILES: O=C(NO)c1ccc(CN(C(=O)N2CC[C@H](F)C2)c2cccc(C(F)(F)F)c2)cc1] |
| 497 | 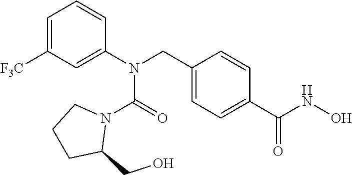 [SMILES: O=C(NO)c1ccc(CN(C(=O)N2CCC[C@@H]2CO)c2cccc(C(F)(F)F)c2)cc1] |
| 498 | 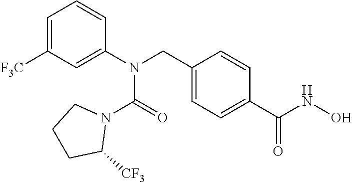 |
| 499 | 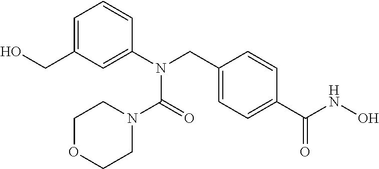 [SMILES: O=C(NO)c1ccc(CN(C(=O)N2CCOCC2)c2cccc(CO)c2)cc1] |
| 500 | 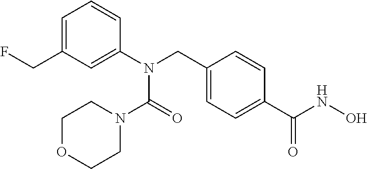 [SMILES: O=C(NO)c1ccc(CN(C(=O)N2CCOCC2)c2cccc(CF)c2)cc1] |
| 511 | 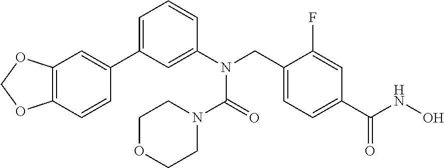 [SMILES: O=C(NO)c1ccc(CN(C(=O)N2CCOCC2)c2cccc(-c3ccc4c(c3)OCO4)c2)c(F)c1] |
| 512 | 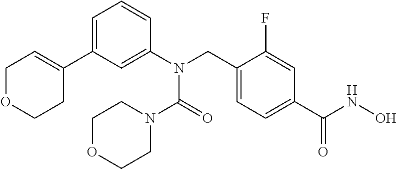 [SMILES: O=C(NO)c1ccc(CN(C(=O)N2CCOCC2)c2cccc(C3=CCOCC3)c2)c(F)c1] |
| 513 | 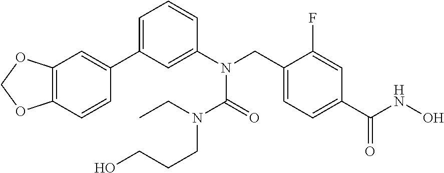 [SMILES: CCN(CCCO)C(=O)N(Cc1ccc(C(=O)NO)cc1F)c1cccc(-c2ccc3c(c2)OCO3)c1] |
| 514 | 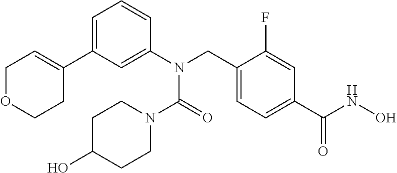 [SMILES: O=C(NO)c1ccc(CN(C(=O)N2CCC(O)CC2)c2cccc(C3=CCOCC3)c2)c(F)c1] |
| 517 | 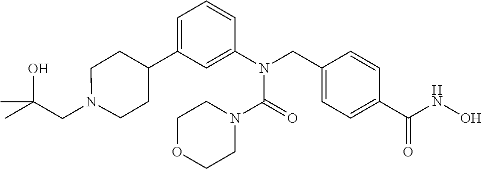 [SMILES: CC(C)(O)CN1CCC(c2cccc(N(Cc3ccc(C(=O)NO)cc3)C(=O)N3CCOCC3)c2)CC1] |
| 518 | 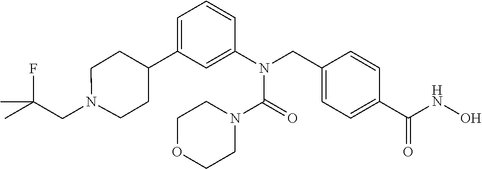 [SMILES: CC(C)(F)CN1CCC(c2cccc(N(Cc3ccc(C(=O)NO)cc3)C(=O)N3CCOCC3)c2)CC1] |
| 520 | 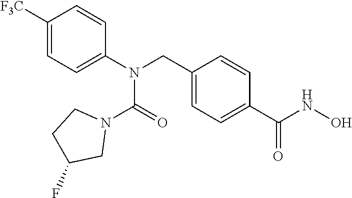 |
| 521 | 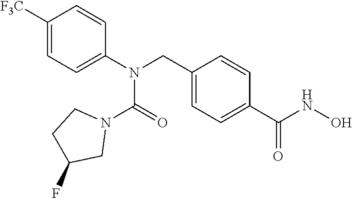 |
| 522 | 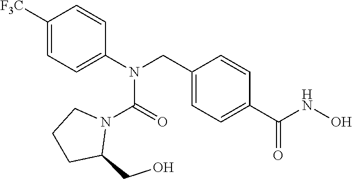 [SMILES: O=C(NO)c1ccc(CN(C(=O)N2CCC[C@@H]2CO)c2ccc(C(F)(F)F)cc2)cc1] |
| 529 | 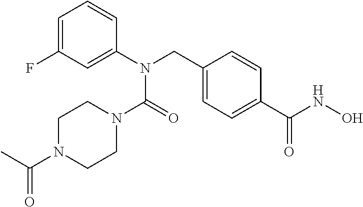 [SMILES: CC(=O)N1CCN(C(=O)N(Cc2ccc(C(=O)NO)cc2)c2cccc(F)c2)CC1] |
| 530 | 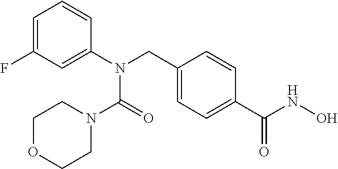 [SMILES: O=C(NO)c1ccc(CN(C(=O)N2CCOCC2)c2cccc(F)c2)cc1] |
| 531 | 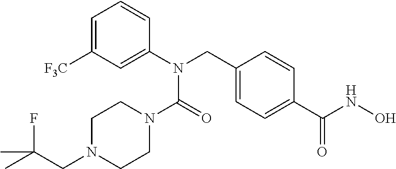 [SMILES: CC(C)(F)CN1CCN(C(=O)N(Cc2ccc(C(=O)NO)cc2)c2cccc(C(F)(F)F)c2)CC1] |
| 532 | 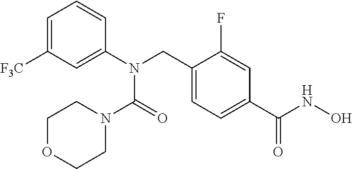 [SMILES: O=C(NO)c1ccc(CN(C(=O)N2CCOCC2)c2cccc(C(F)(F)F)c2)c(F)c1] |
| 533 | 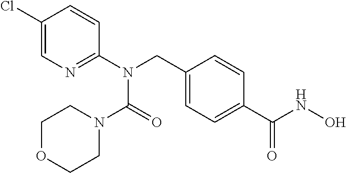 [SMILES: O=C(NO)c1ccc(CN(C(=O)N2CCOCC2)c2ccc(Cl)cn2)cc1] |
| 543 | 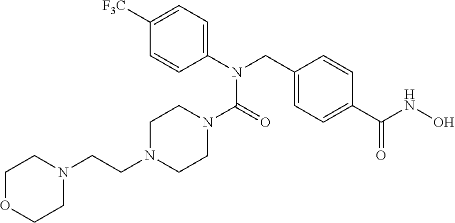 |
| 544 | 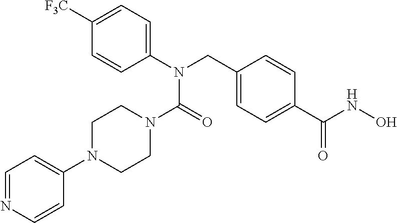 [SMILES: O=C(NO)c1ccc(CN(C(=O)N2CCN(c3ccncc3)CC2)c2ccc(C(F)(F)F)cc2)cc1] |
| 545 | 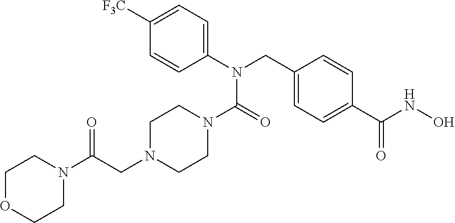 |
| 577 | 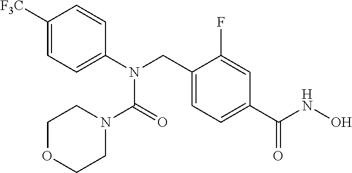 [SMILES: O=C(NO)c1ccc(CN(C(=O)N2CCOCC2)c2ccc(C(F)(F)F)cc2)c(F)c1] |
| 578 | 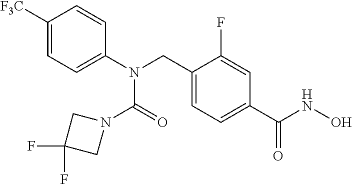 [SMILES: O=C(NO)c1ccc(CN(C(=O)N2CC(F)(F)C2)c2ccc(C(F)(F)F)cc2)c(F)c1] |
| 580 | 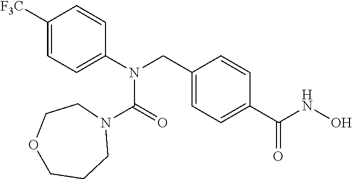 [SMILES: O=C(NO)c1ccc(CN(C(=O)N2CCCOCC2)c2ccc(C(F)(F)F)cc2)cc1] |
| 651 | 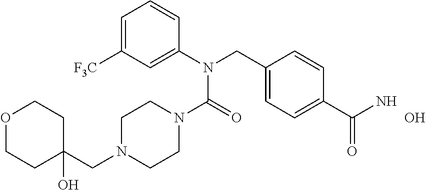 |
| 652 | 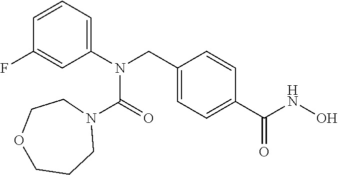 [SMILES: O=C(NO)c1ccc(CN(C(=O)N2CCCOCC2)c2cccc(F)c2)cc1] |
| 683 | 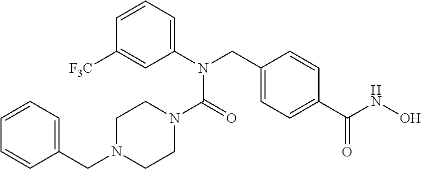 |
| 684 | 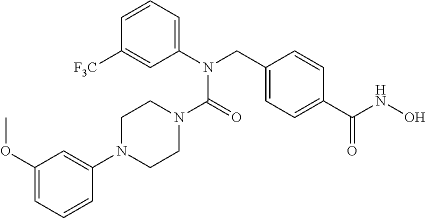 |
| 716 | 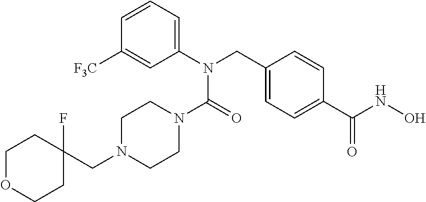 |
| 717 | 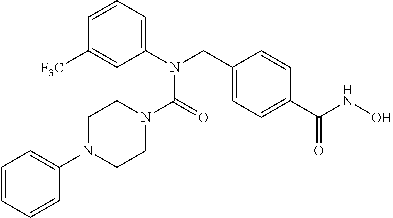 |
| 718 | 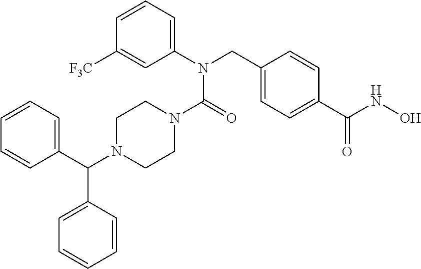 |
| 765 | 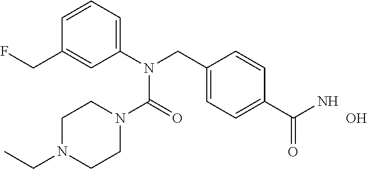 |
| 766 | 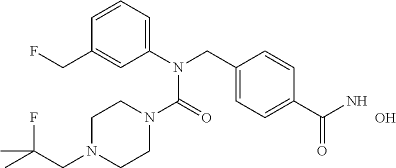 |
| 771 | 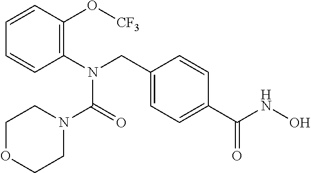 [SMILES: O=C(NO)c1ccc(CN(C(=O)N2CCOCC2)c2ccccc2OC(F)(F)F)cc1] |
| 772 | 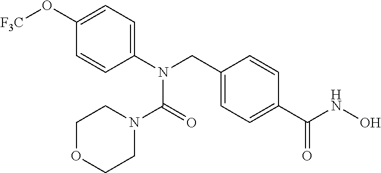 [SMILES: O=C(NO)c1ccc(CN(C(=O)N2CCOCC2)c2ccc(OC(F)(F)F)cc2)cc1] |
| 773 | 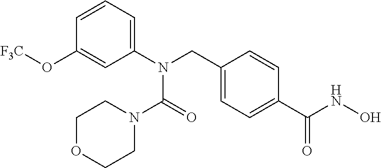 |
| 774 | 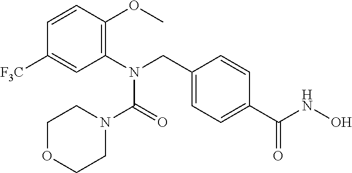 |
| 776 | 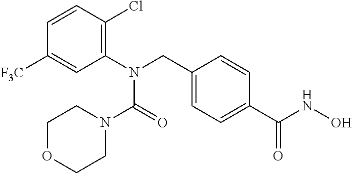 |
| 778 | 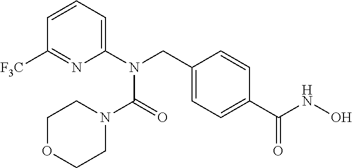 |
| 791 | 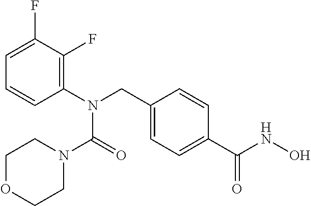 [SMILES: O=C(NO)c1ccc(CN(C(=O)N2CCOCC2)c2cccc(F)c2F)cc1] |
| 797 | 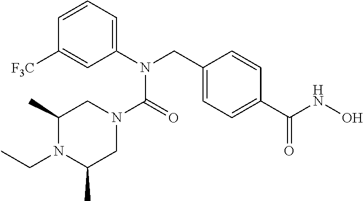 [SMILES: CCN1[C@H](C)CN(C(=O)N(Cc2ccc(C(=O)NO)cc2)c2cccc(C(F)(F)F)c2)C[C@@H]1C] |
| 800 | 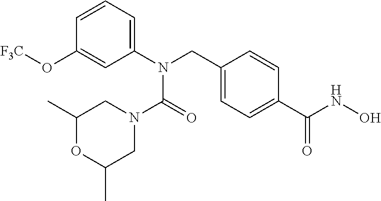 [SMILES: CC1CN(C(=O)N(Cc2ccc(C(=O)NO)cc2)c2cccc(OC(F)(F)F)c2)CC(C)O1] |
| 801 | 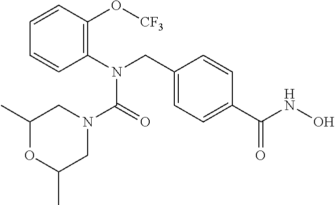 |
| 802 | 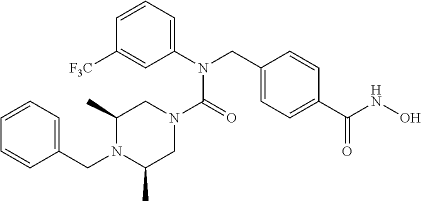 [SMILES: C[C@@H]1CN(C(=O)N(Cc2ccc(C(=O)NO)cc2)c2cccc(C(F)(F)F)c2)C[C@H](C)N1Cc1ccccc1] |
| 803 | 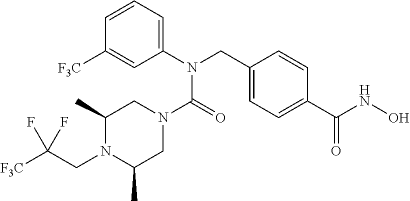 [SMILES: C[C@@H]1CN(C(=O)N(Cc2ccc(C(=O)NO)cc2)c2cccc(C(F)(F)F)c2)C[C@H](C)N1CC(F)(F)C(F)(F)F] |
| 826 | 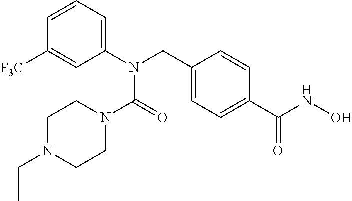 [SMILES: CCN1CCN(C(=O)N(Cc2ccc(C(=O)NO)cc2)c2cccc(C(F)(F)F)c2)CC1] |
| 827 | 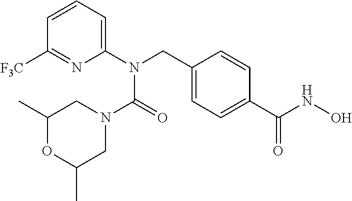 [SMILES: CC1CN(C(=O)N(Cc2ccc(C(=O)NO)cc2)c2cccc(C(F)(F)F)n2)CC(C)O1] |
| 828 | 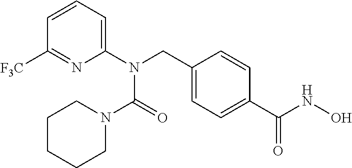 [SMILES: O=C(NO)c1ccc(CN(C(=O)N2CCCCC2)c2cccc(C(F)(F)F)n2)cc1] |
| 829 | 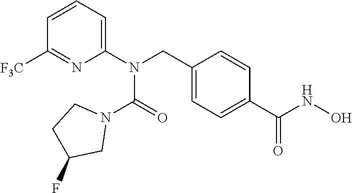 [SMILES: O=C(NO)c1ccc(CN(C(=O)N2CC[C@H](F)C2)c2cccc(C(F)(F)F)n2)cc1] |

5. The pharmaceutical composition according to claim 1, wherein the inflammatory rheumatic diseases are rheumatoid arthritis, degenerative arthritis, reactive arthritis, enteropathic arthritis, septic arthritis, psoriatic arthritis, Reiter's syndrome, osteoarthritis, ankylosing spondylitis, Behcet's disease or lupus.

6. The pharmaceutical composition according to claim 1, wherein the histone deacetylase inhibitor is orally administered.

7. A method for preventing or treating inflammatory rheumatic diseases, including administering a therapeutically effective amount of a composition according to any one of claim 1 to 6.

8. Use of the composition comprising a histone deacetylase inhibitor and methotrexate according to any one of claims 1 to 6, in preparing a drug for preventing or treating inflammatory rheumatic diseases.
